(19) United States
(12) Patent Application Publication
(10) Pub. No.: US 2013/0085161 A1
(43) Pub. Date: Apr. 4, 2013

(54) PIPERIDINYL SUBSTITUTED 1,3-DIHYDRO-BENZOIMIDAZOL-2-YLIDENEAMINE DERIVATIVES

(71) Applicants: Frederic Berst, Altkirch (FR); Pascal Furet, Thann (FR); Andreas Marzinzik, Weil (DE); Frederic Stauffer, Hesingue (FR)

(72) Inventors: Frederic Berst, Altkirch (FR); Pascal Furet, Thann (FR); Andreas Marzinzik, Weil (DE); Frederic Stauffer, Hesingue (FR)

(73) Assignee: NOVARTIS AG, Basel (CH)

(21) Appl. No.: 13/704,380

(22) PCT Filed: Jun. 16, 2011
(86) PCT No.: PCT/EP2011/060032

§ 371 Date: Dec. 14, 2012

Related U.S. Application Data
(60) Provisional application No. 61/355,845, filed on Jun. 17, 2010.

(51) Int. Cl. C07D 401/04; A61K 31/4545; C07D 401/14; A61K 31/454; A61K 45/06

(52) U.S. Cl. CPC C07D 401/04; A61K 31/454; A61K 45/06; C07D 401/14; A61K 31/4545
USPC 514/318; 546/199; 514/322; 546/194

(57) ABSTRACT

The invention relates to new derivatives of formula (I),

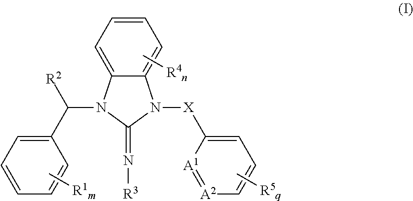

wherein the substituents are as defined in the specification; to processes for the preparation of such derivatives; pharmaceutical compositions comprising such derivatives; such derivatives as a medicament; such derivatives for the treatment of one or more IGF-1R mediated disorders or diseases.

### FIELD OF THE INVENTION

The invention relates to new derivatives of 1H-benzo[d]imidazol-2(3H)-imines; processes for the preparation of such derivatives; pharmaceutical compositions comprising such derivatives optionally in combination with one or more other pharmaceutically active compounds; such derivatives optionally in combination with one or more other pharmaceutically active compounds as a medicament; such derivatives optionally in combination with one or more other pharmaceutically active compounds for the treatment of a proliferative disease, such as a tumour disease (also including a method for the treatment of such diseases in mammals, especially in humans); and the use of such derivatives for the preparation of a pharmaceutical composition (medicament) for the treatment of a proliferative disease, such as a tumour.

### BACKGROUND OF THE INVENTION

Insulin-like growth factor (IGF-1) signaling is highly implicated in cancer, with the IGF-1 receptor (IGF-1R) as the predominating factor. IGR-1R is important for tumor transformation and survival of malignant cells, but is only partially involved in normal cell growth. Targeting of IGF-1R has been suggested to be a promising option for cancer therapy. (Larsson et al., Br. J. Cancer 92:2097-2101 (2005)).

WO 2005/097800 discloses certain 6,6-bicyclic ring substituted heterobicyclic derivatives having therapeutic activity as IGF-1R inhibitors. WO 2005/037836 discloses certain imidazopyrazine derivatives having therapeutic activity as IGF-1R inhibitors. WO2006/074991 discloses certain 1H-benzo[d]imidazol-2(3H)-imines having therapeutic activity as modulators of the SK-channels.

Because of the emerging disease-related roles of IGF-1R, there is a continuing need for compounds which may be useful for treating and preventing a disease which responds to inhibition of IGF-1R, particularly for compounds with improved efficacy, tolerabilty and/or selectivity. They should be well absorbed from the gastrointestinal tract, be metabolically stable and possess favourable pharmacokinetic properties. They should be non-toxic and demonstrate few side-effects. Furthermore, the ideal drug candidate will exist in a physical form that is stable, non-hygroscopic and easily formulated.

### SUMMARY OF THE INVENTION

The present invention relates to new derivatives of 1H-benzo[d]imidazol-2(3H)-imines of formula (I)

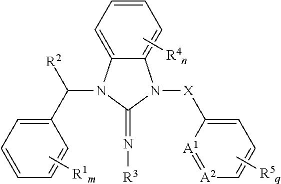

or a salt thereof, wherein R¹-R⁵, A¹, A², X, m, n, and q are defined below. The invention also relates to processes for the preparation of such derivatives; pharmaceutical compositions comprising such derivatives optionally in combination with one or more other pharmaceutically active compounds; such derivatives optionally in combination with one or more other pharmaceutically active compounds as a medicament; such derivatives optionally in combination with one or more other pharmaceutically active compounds for the treatment of a proliferative disease, such as a tumour disease (also including a method for the treatment of such diseases in mammals, especially in humans); and the use of such derivatives for the preparation of a pharmaceutical composition (medicament) for the treatment of a proliferative disease, such as a tumour.

### DETAILED DESCRIPTION OF THE INVENTION

The invention therefore provides in a first aspect a compound of formula (I),

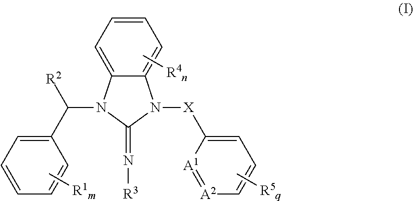

or a salt thereof, wherein
m represents 0, 1, 2, 3 or 4;
n represents 0, 1, 2, 3 or 4;
q represents 0, 1, 2 or 3;
X represents a group

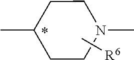

wherein the atom marked * is bound to the imidazole;
A¹ represents N, CH or CR⁵;
A² represents N, CH or CR⁵;
R¹ represents halogen, C₁₋₇alkyl, C₁₋₇alkyoxy, halo-C₁₋₇alkyl or halo-C₁₋₇alkyoxy; and/or
R¹ represents, provided two substituents R¹ are in vicinal position, together with the carbon atoms to which they are attached a cyclic moiety, said moiety (a) being saturated or partly saturated, (b) contains 5-8 ring forming atoms, (c) contains 0-3 nitrogen atoms, 0-2 oxygen atoms, 0-2 sulfur atoms, and (d) is unsubstituted or substituted, the substituents being selected from the group consisting of halogen, C₁₋₇alkyl, C₁₋₇alkyoxy, halo-C₁₋₇alkyl and halo-C₁₋₇alkyoxy;
R² represents hydrogen, halogen, C₁₋₇alkyl or halo-C₁₋₇alkyl;
R³ represents hydrogen, C₁₋₇alkyl, halo-C₁₋₇alkyl, C₁₋₇alkyl-carbonyl, halo-C₁₋₇alkyl-carbonyl, C₁₋₇alkoxy-carbonyl, or halo-C₁₋₇alkoxy-carbonyl;
R⁴ represents halogen, C₁₋₇alkyl, C₁₋₇alkoxy, halo-C₁₋₇alkyl or halo-C₁₋₇alkoxy;
R⁵ represents a substituent different from hydrogen, said substituent (a) having 1-50 atoms selected from the group consisting of hydrogen, carbon, halogen and hetero atoms and (b) being bound via a single bond;
R⁶ represents hydrogen, hydroxy, halogen, C₁₋₇alkyl, C₁₋₇alkyoxy, halo-C₁₋₇alkyl or halo-C₁₋₇alkyoxy.

It has been found that the compounds of formula (I) are potent inhibitors of the tyrosine kinase activity of the Insulin-like growth factor I receptor (IGF-IR) and inhibit IGF-IR-dependent cell proliferation. The pesence of the substituents of the scaffold as defined below is considered important for the efficacy, tolerability and/or the selectivty of the compounds of the present invention as IGF-IR tyrosine kinase inhibitors and their potential to inhibit IGF-IR-dependent cell proliferation.

The compounds of the present invention are therefore potentially useful in the treatment of a wide range of disorders, particularly the treatment of proliferative diseases. The compounds of formula (I) therefore permit, for example, a therapeutic approach, especially for diseases in the treatment of which, and also for the prevention of which, an inhibition of the IGF-IR tyrosine kinase and/or of the IGF-IR-dependent cell proliferation shows beneficial effects. Such diseases include proliferative diseases, such as tumours, like for example breast, renal, prostate, colorectal, thyroid, ovarian, pancreas, neuronal, lung, uterine and gastro-intestinal tumours as well as osteosarcomas and melanomas. Compounds of the invention show improved efficacy, tolerability and/or selectivity when compared to known IGF-1R inhibitors. Without being bound to theory, it is believed that several factors contribute to the improvements in efficacy and tolerability, for example increased metabolic stability and the reduced formation of multiple kinase-active metabolites. Although known compounds have been shown to produce desirable effects in in-vivo models through the inhibition of IGF-1 receptor activity, they have been found to undergo extensive metabolism. This not only limits the pharmacokinetic profile of such derivatives, but also generates metabolites, which show multiple potent kinase activities.

The invention may be more fully appreciated by reference to the following description, including the following glossary of terms and the concluding examples. As used herein, the terms “including”, “containing” and “comprising” are used herein in their open, non-limiting sense. Where the plural form is used for compounds, salts, and the like, this is taken to mean also a single compound, salt, or the like.

Unless specified otherwise, the term “compounds of the present invention” refers to compounds of formula (I) and subformulae thereof (add other additional genus structures as necessary), prodrugs thereof, salts of the compound and/or prodrugs, hydrates or solvates of the compounds, salts and/or prodrugs, as well as all stereoisomers (including diastereoisomers and enantiomers), tautomers and isotopically labeled compounds (including deuterium substitutions), as well as inherently formed moieties (e.g., polymorphs, solvates and/or hydrates).

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S.

Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers or axes and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% ee in the (R)- or (S)-configuration.

Substituents at atoms with unsaturated bonds may be present in cis-(Z)- or trans-(E) form. Particularly, R³ may be present in cis-form, trans-form or mixtures thereof. Particularly, R⁶ may be present in cis-form, trans-form or mixtures thereof, whereby cis/trans relates to the relative position of R⁶ in relation to the core benzimidazole-moiety.

Accordingly, as used herein, a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulformate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Compounds of the present invention are either obtained in the free form, as a salt thereof, or as prodrug derivatives thereof. When both a basic group and an acid group are present in the same molecule, the compounds of the present invention may also form internal salts, e.g., zwitterionic molecules.

The present invention also provides pro-drugs of the compounds of the present invention that converts in vivo to the compounds of the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound that contain one or more protective groups and are converted to an active form by metabolism or solvolysis. Both the active drug form and any released metabolic products should have acceptably low toxicity.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released trans-port moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxylcarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water. The compounds of the present invention, including salts, hydrates and solvates thereof, may inherently or by design form polymorphs.

Compounds of the present invention that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H, ¹³C, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed. Pharmaceutically acceptable solvates in accordance with the invention include those wherein the solvent of crystallization may be isotopically substituted, e.g. D₂O, d₆-acetone, d₆-DMSO.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula (where one or more up to all more general expressions in embodiments characterized as preferred above or below can be replaced with a more specific definition, thus leading to a more preferred embodiment of the invention, respectively).

Carbon containing groups, moieties or molecules contain 1 to 12, preferably 1 to 7, more preferably 1 to 4, most preferably 1 or 2, carbon atoms. Any non-cyclic carbon containing group or moiety with more than 1 carbon atom is straight-chain or branched. The prefix “lower” denotes a radical having 1 to 7, preferably 1 to 4 carbon atoms, the radicals in question being either unbranched or branched with single or multiple branching.

As used herein, the term “halogen” (or halo) denotes fluorine, bromine, chlorine or iodine, in particular fluorine, chlorine. Halogen-substituted groups and moieties, such as alkyl substituted by halogen (haloalkyl) can be mono-, poly- or per-halogenated.

As used herein, the term “hetero atoms” denotes atoms other than Carbon and Hydrogen, preferably nitrogen (N), oxygen (O) or sulfur (S), in particular nitrogen or oxygen.

As used herein, the term “alkyl” refers to a fully saturated branched or unbranched hydrocarbon moiety having up to 20 carbon atoms. Unless otherwise provided, alkyl refers to hydrocarbon moieties having 1 to 16 carbon atoms, 1 to 10 carbon atoms, 1 to 7 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like. A substituted alkyl is an alkyl group containing one or more, such as one, two or three substituents as defined herein.

As used herein, the term “alkylene” refers to divalent alkyl group as defined herein above having 1 to 20 carbon atoms. It comprises 1 to 20 carbon atoms, Unless otherwise provided, alkylene refers to moieties having 1 to 16 carbon atoms, 1 to 10 carbon atoms, 1 to 7 carbon atoms, or 1 to 4 carbon atoms. Representative examples of alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene, n-decylene and the like. A substituted alkylene is an alkylene group containing one or more, such as one, two or three substituents as defined herein.

As used herein, the term “haloalkyl” refers to an alkyl as defined herein, which is substituted by one or more halo groups as defined herein. The haloalkyl can be monohaloalkyl, dihaloalkyl or polyhaloalkyl including perhaloalkyl. A monohaloalkyl can have one iodo, bromo, chloro or fluoro within the alkyl group. Dihaloalky and polyhaloalkyl groups can have two or more of the same halo atoms or a combination of different halo groups within the alkyl. Typically the polyhaloalkyl contains up to 12, or 10, or 8, or 6, or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms.

As used herein, the term “alkoxy” refers to alkyl-O—, wherein alkyl is defined herein above. Representative examples of alkoxy include, but are not limited to, methoxy, ethoxy, propoxy, 2-propoxy, butoxy, tert-butoxy, pentyloxy, hexyloxy, cyclopropyloxy-, cyclohexyloxy- and the like. Typically, alkoxy groups have 1-16, 1-10, 1-7, more preferably 1-4 carbon atoms. A substituted alkoxy is an alkoxy group containing one or more, such as one, two or three substituents as defined herein; preferably halo.

Similarily, each alkyl part of other groups like “alkylaminocrabonyl”, “alkoxyalkyl”, “alkoxylcarbonyl”, “alkoxy-carbonylalkyl”, “alkylsulfonyl”, “alkylsulfoxyl”, “alkylamino”, “haloalkyl” shall have the same meaning as described in the above-mentioned definition of “alkyl”.

As used herein, the term “cycloalkyl” refers to saturated or unsaturated monocyclic, bicyclic, tricyclic or spirocyclic hydrocarbon groups of 3-12 carbon atoms. Unless otherwise provided, cycloalkyl refers to cyclic hydrocarbon groups having between 3 and 9 ring carbon atoms or between 3 and 7 ring carbon atoms. A substituted cycloalkyl is a cycloylkyl group containing one or more substituents as defined herein. Preferably, a substituted cycloalkyl is a cycloalkyl group substituted by one, or two, or three, or more substituents independently selected from the group consisting of alkyl, halo, oxo, hydroxy, alkoxy, alkyl-C(O)—, acylamino, carbamoyl, alkyl-NH—, (alkyl)2N—, thiol, alkyl-S—, nitro, cyano, carboxy, alkyl-O—C(O)—, sulfonyl, sulfonamido, sulfamoyl, and heterocyclyl. Exemplary monocyclic hydrocarbon groups include, but are not limited to, cyclopropyl, cyclobutyl, cyclopentyl, cyclopentenyl, cyclohexyl and cyclohexenyl and the like. Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like. Exemplary tricyclic hydrocarbon groups include adamantyl and the like.

Similarily, each cycloalkyl part of other groups like “cycloalkyloxy”, “cycloalkoxyalkyl”, “cycloalkoxycarbonyl”, “cycloalkoxy-carbonylalkyl”, “cycloalkylsulfonyl”, “halocycloalkyl” shall have the same meaning as described in the above-mentioned definition of “alkyl”.

As used herein, the term “aryl” refers to an aromatic hydrocarbon group having 6-20 carbon atoms in the ring portion. Typically, aryl is monocyclic, bicyclic or tricyclic aryl having 6-20 carbon atoms. Furthermore, the term “aryl” as used herein, refers to an aromatic substituent which can be a single aromatic ring, or multiple aromatic rings that are fused together. Non-limiting examples include phenyl, naphthyl or tetrahydronaphthyl. A substituted aryl is an aryl group containing one or more substituents as defined herein. Preferably, a substituted aryl is an aryl group substituted by 1-5 (such as one, or two, or three) substituents independently selected from the group consisting of alkyl, haloalkyl, cycloalkyl, halogen, hydroxy, alkoxy, acyl, alkyl-C(O)—O—, aryloxy, heteroaryloxy-, amino, thiol, alkylthio, arylthio-, nitro, cyano, carboxy, alkyl-O—C(O)—, carbamoyl, alkyl-S(O)—, sulfonyl, sulfonamido, aryl and heterocyclyl.

Similarily, each aryl part of other groups like “aryloxy”, “aryloxyalkyl”, “aryloxycarbonyl”, “aryloxy-carbonylalkyl” shall have the same meaning as described in the above-mentioned definition of “aryl”.

As used herein, the term “heterocyclyl” refers to a heterocyclic radical that saturated or partially saturated and is preferably a monocyclic or a polycyclic ring (in case of a polycyclic ring particularly a bicyclic, tricyclic or spirocyclic ring); and has 3 to 24, more preferably 4 to 16, most preferably 5 to 10 and most preferably 5 or 6 ring atoms; wherein one or more, preferably one to four, especially one or two ring atoms are a heteroatom (the remaining ring atoms therefore being carbon). The bonding ring (i.e. the ring connecting to the molecule) preferably has 4 to 12, especially 5 to 7 ring atoms. The term heterocyclyl excludes heteroaryl. The heterocyclic group can be attached at a heteroatom or a carbon atom. The heterocyclyl can include fused or bridged rings as well as spirocyclic rings. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, and the like. A substituted heterocyclyl is a heterocyclyl group containing one or more substituents as defined herein. Preferably, a substituted heterocyclyl is an heterocyclyl group substituted by 1-5 (such as one, or two, or three) substituents independently selected from the group consisting of the substituents defined above for substituted alkyl and/or from one or more of the following substituents: alkyl, oxo (═O), thiono (═S), imino(═NH), imino-alkyl.

Similarily, each heterocyclyl part of other groups like “heterocyclyloxy”, “heterocyclyloxyalkyl”, “heterocyclyloxycarbonyl” shall have the same meaning as described in the above-mentioned definition of “heterocyclyl”.

As used herein, the term “heteroaryl” refers to a 5-14 membered monocyclic- or bicyclic- or tricyclic-aromatic ring system, having 1 to 8 heteroatoms. Typically, the heteroaryl is a 5-10 membered ring system (e.g., 5-7 membered monocycle or an 8-10 membered bicycle) or a 5-7 membered ring system. Typical heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, and 2-, 4-, or 5-pyrimidinyl. The term “heteroaryl” also refers to a group in which a heteroaromatic ring is fused to one or more aryl, cycloaliphatic, or heterocyclyl rings, where the radical or point of attachment is on the heteroaromatic ring. Nonlimiting examples include 1-, 2-, 3-, 5-, 6-, 7-, or 8-indolizinyl, 1-, 3-, 4-, 5-, 6-, or 7-isoindolyl, 2-, 3-, 4-, 5-, 6-, or 7-indolyl, 2-, 3-, 4-, 5-, 6-, or 7-indazolyl, 2-, 4-, 5-, 6-, 7-, or 8-purinyl, 1-, 2-, 3-, 4-, 6-, 7-, 8-, or 9-quinolizinyl, 2-, 3-, 4-, 5-, 6-, 7-, or 8-quinoliyl, 1-, 3-, 4-, 5-, 6-, 7-, or 8-isoquinoliyl, 1-, 4-, 5-, 6-, 7-, or 8-phthalazinyl, 2-, 3-, 4-, 5-, or 6-naphthyridinyl, 2-, 3-, 5-, 6-, 7-, or 8-quinazolinyl, 3-, 4-, 5-, 6-, 7-, or 8-cinnolinyl, 2-, 4-, 6-, or 7-pteridinyl, 1-, 2-, 3-, 4-, 5-, 6-, 7-, or 8-4-aH carbazolyl, 1-, 2-, 3-, 4-, 5-, 6-, 7-, or 8-carbzaolyl, 1-, 3-, 4-, 5-, 6-, 7-, 8-, or 9-carbolinyl, 1-, 2-, 3-, 4-, 6-, 7-, 8-, 9-, or 10-phenanthridinyl, 1-, 2-, 3-, 4-, 5-, 6-, 7-, 8-, or 9-acridinyl, 1-, 2-, 4-, 5-, 6-, 7-, 8-, or 9-perimidinyl, 2-, 3-, 4-, 5-, 6-, 8-, 9-, or 10-phenathrolinyl, 1-, 2-, 3-, 4-, 6-, 7-, 8-, or 9-phenazinyl, 1-, 2-, 3-, 4-, 6-, 7-, 8-, 9-, or 10-phenothiazinyl, 1-, 2-, 3-, 4-, 6-, 7-, 8-, 9-, or 10-phenoxazinyl, 2-, 3-, 4-, 5-, 6-, or I-, 3-, 4-, 5-, 6-, 7-, 8-, 9-, or 10-benzisoqinolinyl, 2-, 3-, 4-, or thieno[2,3-b]furanyl, 2-, 3-, 5-, 6-, 7-, 8-, 9-, 10-, or 11-7H-pyrazino[2,3-c]carbazolyl, 2-, 3-, 5-, 6-, or 7-2H-furo[3,2-b]-pyranyl, 2-, 3-, 4-, 5-, 7-, or 8-5H-pyrido[2,3-d]-o-oxazinyl, 1-, 3-, or 5-1H-pyrazolo[4,3-d]-oxazolyl, 2-, 4-, or 54H-imidazo[4,5-d]thiazolyl, 3-, 5-, or 8-pyrazino[2,3-d]pyridazinyl, 2-, 3-, 5-, or 6-imidazo[2,1-b]thiazolyl, 1-, 3-, 6-, 7-, 8-, or 9-furo[3,4-c]cinnolinyl, 1-, 2-, 3-, 4-, 5-, 6-, 8-, 9-, 10, or 11-4H-pyrido[2,3-c]carbazolyl, 2-, 3-, 6-, or 7-imidazo[1,2-b][1,2,4]triazinyl, 7-benzo[b]thienyl, 2-, 4-, 5-, 6-, or 7-benzoxazolyl, 2-, 4-, 5-, 6-, or 7-benzimidazolyl, 2-, 4-, 4-, 5-, 6-, or 7-benzothiazolyl, 1-, 2-, 4-, 5-, 6-, 7-, 8-, or 9-benzoxapinyl, 2-, 4-, 5-, 6-, 7-, or 8-benzoxazinyl, 1-, 2-, 3-, 5-, 6-, 7-, 8-, 9-, 10-, or 11-1H-pyrrolo[1,2-b][2]benzazapinyl. Typical fused heteroary groups include, but are not limited to 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, and 2-, 4-, 5-, 6-, or 7-benzothiazolyl. A substituted heteroaryl is a heteroyryl group containing one or more substituents as defined herein. Preferably, a substituted heteroyryl is a heteroyryl group substituted by 1-5 (such as one, or two, or three) substituents independently selected from the group consisting of the substituents defined above for substituted alkyl and/or from one or more of the following substituents: alkyl, oxo (═O), thiono (═S), imino(═NH), imino-alkyl.

Similarily, each heteroaryl part of other groups like “heteroaryloxy”, “heteroaryloxyalkyl”, “heteroaryloxycarbonyl” shall have the same meaning as described in the above-mentioned definition of “heteroaryl”.

As used herein, the term “substituted” or “a substituent different from hydrogen” refers to a moity that is substituted by one or more, typically 1, 2, 3 or 4, covalently bound suitable non-hydrogen substituents; said substituent containing 1-50 atoms selected from the group consisting of hydrogen, carbon, halogen and hetero atoms. Preferably, non-hydrogen substituents are each independently selected from the group consisting of:
(a) halo, nitro, cyano;
(b) oxo (═O), carboxyl (COOH), formyl (CHO), carbamoyl (CONH₂);
(c) mercapto (SH), sulfinyl (S(O)), sulfonyl (S(O₂)), sulfoxy (S(O)), sulfamoyl (SO₂NH₂), sulfonamido (e.g. SO₂N(H)C₁₋₇alkyl);
(d) alkyl, cycloalkyl; aryl, heterocyclyl, heteroaryl;
(e) hydroxy, alkoxy, cycloalkoxy, aryloxy, heterocyclyoxy, heteroaryloxy;
(f) alkyl-S—, cycloalkyl-S—, aryl-S—; heterocyclyl-S—, heteroaryl-S—;
(g) cycloalkyl-alkyl, aryl-alkyl, heterocyclyl-alkyl, heteroaryl-alkyl;
(h) amino, alkylamino, dialkylamino, cycloylkylamino, arylamino, heterocyclyolamino, heteroarylamino;
(i) alkyl-C(O)—O—, cycloalkyl-C(O)—O—, aryl-C(O)—O—; heterocyclyl-C(O)—O—, heteroaryl-C(O)—O—;
(j) alkyl-O—C(O)—; cycloalkyl-O—C(O)—, aryl-O—C(O)—; heterocyclyl-O—C(O)—, heteroaryl-O—C(O)—;
(k) alkyl-C(O)—NH—, cycloalkyl-C(O)—NH—, aryl-C(O)—NH—; heterocyclyl-C(O)—NH—, heteroaryl-C(O)—NH—;
(l) alkyl-NH—C(O)—; cycloalkyl-NH—C(O)—, aryl-NH—C(O)—; heterocyclyl-NH—C(O)—, heteroaryl-NH—C(O)—;

wherein each cycloalkyl, aryl, heterocyclyl, heteroaryl may be substituted with halogen, hydroxy, alkyl, alkoxy, haloalkyl, haloakloxy, cycloalkyl, amino, alkylamino, dialkylamino alkyl-C(O)—NH—, alkyl-NH—C(O)—, as defined herein and wherein each alkyl may be substituted with halogen, hydroxy, alkoxy, haloalkyl, haloakloxy, cycloalkyl, amino, alkylamino, dialkylamino alkyl-C(O)—NH—, alkyl-NH—C(O)—, as defined herein and wherein each sulfonyl, sulfoxy, sulfamoyl, sulfonamido may be substituted with alkyl, cycloalkyl, aryl, heterocyclyl, heteroaryl.

In preferred embodiments, which are preferred independently, collectively or in any combination or sub-combination, the invention relates to a compound of the formula (I), wherein the substituents are as defined herein.

The invention further relates to pharmaceutically acceptable prodrugs of a compound of formula (I). Particularly, the present invention also relates to pro-drugs of a compound of formula (I) as defined herein that convert in vivo to the compound of formula (I) as such.

The invention further relates to pharmaceutically acceptable metabolites of a compound of formula (I).

Various embodiments of the invention are described herein. It will be recognized that features specified in each embodiment may be combined with other specified features to provide further embodiments.

Consequently, in one embodiment, the invention provides a compound of the formula (I), or a salt thereof, depicted by formula (I-1)

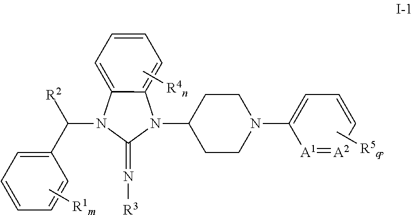

wherein the substituents are as defined herein.

In a further embodiment the invention provides a compound of the formula (I), or a salt thereof, depicted by formula (I-2)

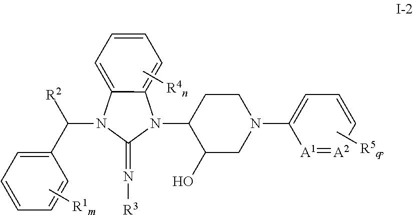

wherein the substituents are as defined herein.

In a further embodiment the invention provides a compound of the formula (I), or a salt thereof, depicted by formula (I-3)

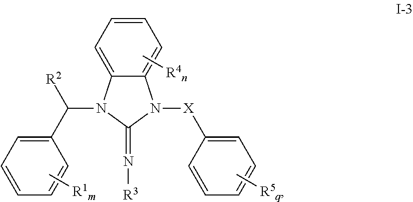

wherein the substituents are as defined herein.

In a further embodiment the invention provides a compound of the formula (I), or a salt thereof, depicted by formula (I-4)

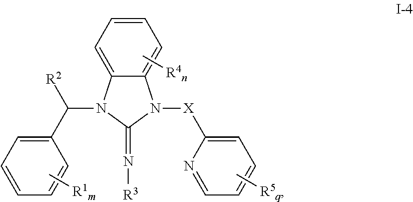

wherein the substituents are as defined herein.

In a further embodiment the invention provides a compound of the formula (I), or a salt thereof, depicted by formula (I-5)

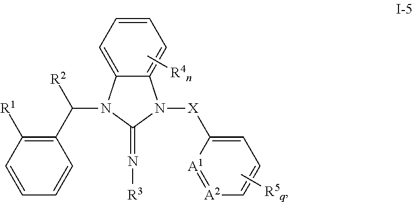

wherein the substituents are as defined herein.

In a further embodiment the invention provides a compound of the formula (I), or a salt hereof, depicted by formula (I-6)

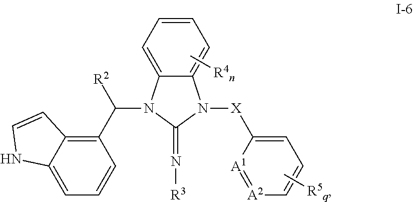

wherein the substituents are as defined herein.

In a further embodiment the invention provides a compound of the formula (I), or a salt thereof, depicted by formula (I-7)

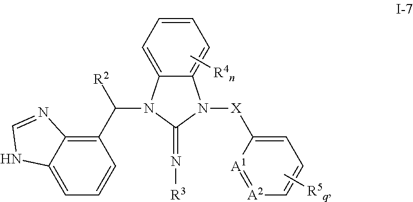

wherein the substituents are as defined herein.

In a further embodiment the invention provides a compound of the formula (I), or a salt thereof, depicted by formula (I-8)

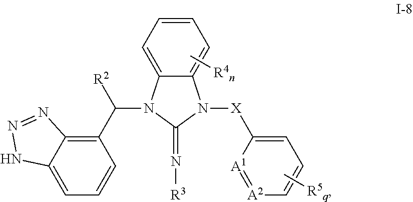

wherein the substituents are as defined herein.

In a further embodiment the invention provides a compound of the formula (I), or a salt thereof, depicted by formula (I-9)

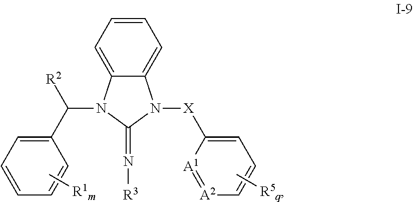

wherein the substituents are as defined herein.

In a further embodiment the invention provides a compound of the formula (I), or a salt thereof, depicted by formula (I-10)

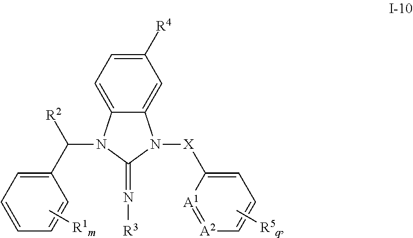

wherein the substituents are as defined herein.

In a further embodiment, m represents 0, 1, 2 or 3; particularly 1 or 2.

In a further embodiment, n represents 0, or 1; particularly 0.

In a further embodiment, q represents 0, 1 or 2; particularly 1 or 2.

In a further embodiment, q represents 2, the substituents R⁵ being located in the 2- and 5-position.

In a further embodiment, q represents 1, the substituent R⁵ being located in the 2- or 3-position.

In a further embodiment, R¹ represents halogen; particularly fluoro or chloro.

In a further embodiment, R¹ represents, together with the phenyl ring, an unsubstituted or substituted indolyl, isoindolyl, indazolyl, benzimidazolyl, benztriazolyl, chinolinyl, isochinnolinyl, cinnolinyl, phtalazinyl, chazolinyl, chinoxalinyl, naphtalenyl, tetrahydro-naphtalenyl, indenyl, dihydro-indenyl, the substituents being selected from the group consisting of halogen.

In a further embodiment, R¹ represents, together with the phenyl ring, an unsubstituted or substituted indolyl, benzimidazolyl, benztriazolyl, the substituents being selected from the group consisting of fluoro and chloro.

In a further embodiment, R² represents hydrogen or C₁₋₇alkyl; particularly hydrogen.

In a further embodiment, R³ represents hydrogen, C₁₋₇alkyl-carbonyl or C₁₋₇alkyloxy-carbonyl; particularly hydrogen or acetyl.

In a further embodiment, R⁴ represents halogen; particularly fluoro.

In a further embodiment, R⁵ represents a group —X′—R⁵′ wherein

X′ represents either a single bond or a linker selected from the group consisting of

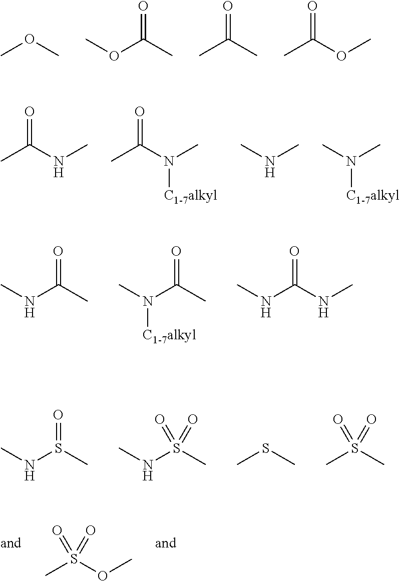

R⁵′ represents hydroxy, halo, cyano, carboxy, aminocarbonyl (CONH2), amino, or optionally substituted C₁₋₇alkyl, optionally substituted C₃₋₁₂cycloalkyl, optionally substituted C₆₋₂₀aryl, optionally substituted heterocyclyl having 3-24 ring atoms, optionally substituted heteroaryl having 5-14 ring atoms, the optional substituents being selected from the group consisting of hydroxy, halo, cyano, carboxy, aminocarbonyl, amino, C₁₋₇alkylamino, di(C₁₋₇alkyl)amino, C₁₋₇alkyl, C₁₋₇alkyloxy.

In a further embodiment, R⁵ represents a group —X′—R⁵′ wherein

X′ represents a single bond and

R⁵′ represents hydroxy, halo, cyano, carboxy, aminocarbonyl (CONH₂), amino, C₁₋₇alkyl or substituted C₁₋₇alkyl, the substituents being selected from the group consisting of hydroxy, halo, amino, C₁₋₇alkylamino, C₁₋₇alkyloxy.

In a further embodiment, R⁵ represents a group —X′—R⁵′ wherein

X′ represents a linker selected from the group consisting of

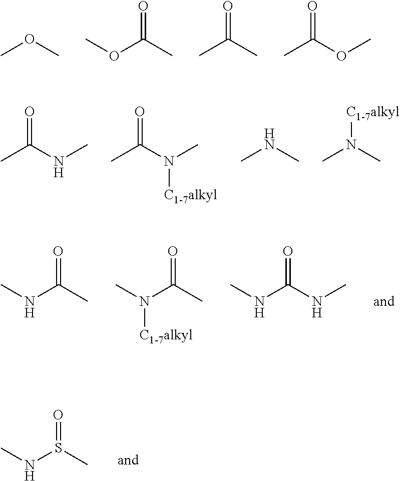

R⁵′ represents optionally substituted C₁₋₄alkyl, optionally substituted C₃₋₉cycloalkyl, optionally substituted C₆₋₁₀aryl, optionally substituted heterocyclyl having 4-16 ring atoms, optionally substituted heteroaryl having 5-10 ring atoms, the optional substituents being selected from the group consisting of hydroxy, halo, cyano, carboxy, amino-carbonyl, amino, C₁₋₇alkylamino, di(C₁₋₇alkyl)amino, C₁₋₇alkyl.

In a further embodiment, R⁵ represents methyl, methoxy, acetylamino, chloro, cyano, trifluoromethyl.

In a further embodiment, R⁶ represents hydrogen, hydroxy, C₁₋₇alkyoxy or halo-C₁₋₇ alkyoxy; particularly hydrogen or hydroxy.

In a further embodiment, A¹ represents N or CR⁵; particularly CR⁵.

In a further embodiment, A² represents CH or CR⁵; particularly CH.

In a very particularly advantageous embodiment, the present invention relates to a compound of formula (I) mentioned in the Examples below, or a salt, especially a pharmaceutically acceptable salt, thereof.

The invention relates in a second aspect to the manufacture of a compound of formula (I). The compounds of formula (I) or salts thereof are prepared in accordance with processes known per se (see references cited above), though not previously described for the manufacture of the compounds of the formula (I).

General Reaction Processes:

In one embodiment, the invention relates to a process for manufacturing a compound of formula (I) wherein R³ represents hydrogen, said method comprising the step of reacting a compound of formula (II)

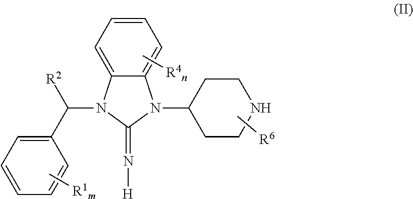

wherein the substituents are as defined above, with a compound of formula (III),

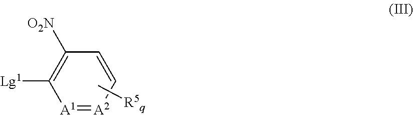

wherein the substituents are as defined above, and Lg¹ represents a suitable leaving group, such as halogen (e.g. fluoro or chloro); optionally in the presence of one or more reaction aids, such as an organic or inorganic base (e.g. NEt3, diisopropylethylamine, Na₂CO₃, Cs₂CO₃, K₂CO₃); optionally in the presence of one or more diluents, particular polar solvents (e.g. DMF, THF, MeCN, NMP). This type of reaction is also known as nucleophilic aromatic substitution, typical reaction conditions are known in the field and may applied to the present process. The compounds of formula (I) obtained by this method contain a nitro group R⁵ and optionally one or more other substituents R⁵. Such nitro group may be removed or converted into other groups according to standard methods in one or more subsequent reaction steps.

In a further embodiment, the invention relates to a process for manufacturing a compound of formula (I) wherein R³ represents hydrogen, said method comprising the step of reacting a compound of formula (IX)

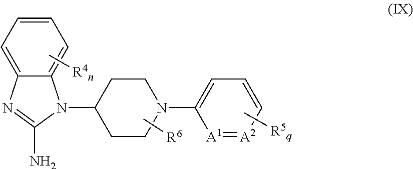

wherein the substituents are as defined above with a compound of formula (V),

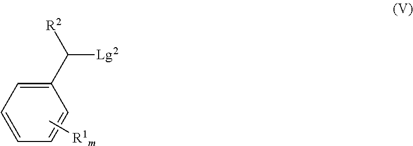

wherein the substituents are as defined above, and Lg² represents a suitable leaving group, such halogen (e.g. bromo, chloro, iodo); optionally in the presence of one or more reaction aids, such as a base (e.g. Na₂CO₃) or an inorganic salt (e.g. KI); optionally in the presence of one or more diluents, particularly polar solvents (e.g. water, MeCN). This type of reaction is also known as alkylation reaction, typical reaction conditions are known in the field and may be applied to the present process. The compounds of formula (I) obtained by this method may contain a nitro group R⁵ and optionally one or more other substituents R⁵. Such nitro group may be removed or converted into other groups according to standard methods in one or more subsequent reaction steps.

In a further embodiment, the invention relates to a process for manufacturing a compound of formula (I) wherein R³ represents a substituent as defined herein except hydrogen, said method comprising the step of reacting a compound of formula (XIII)

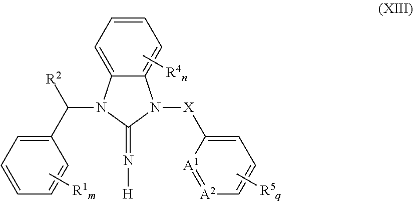

wherein the substituents are as defined herein, with a compound of formula (XIV),

Lg⁵-R³′ (XIV)

wherein R³ represents as substituent as defined herein for R³ except hydrogen and Lg⁵ represents a suitable leaving group, such as halogen (e.g. chloro, fluoro, bromo); optionally in the presence of one or more reaction aids, such as an organic or inorganic base (e.g. NEt3, diisopropylethylamine, Na₂CO₃, Cs₂CO₃, K₂CO₃); optionally in the presence of one or more diluents, particularly one or more polar solvents (e.g. Ethyl acetate, dichloromethane, DMF, NMP, THF). This type of reaction is also known as acylation (in case R³ represents alkyl-carbonyl) or alkylation (in case R³ represents alkyl) typical reaction conditions are known in the field and may applied to the present process.

Starting Materials

New starting materials and/or intermediates, as well as processes for the preparation thereof, are likewise the subject of this invention. In the preferred embodiment, such starting materials are used and reaction conditions so selected as to enable the preferred compounds to be obtained.

In one embodiment, the invention relates to a process for manufacturing a compound of formula (II),

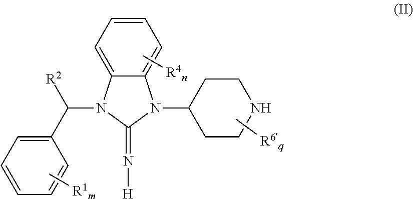

wherein the substituents are as defined above; said method comprising the step of reacting a compound of formula (IV)

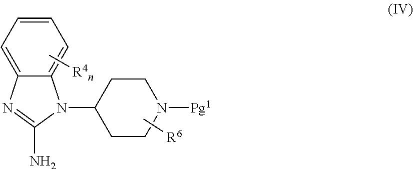

wherein the substituents are as defined above, and Pg¹ represents a suitable protecting group (e.g. BOC) or hydrogen, with a compound of formula (V),

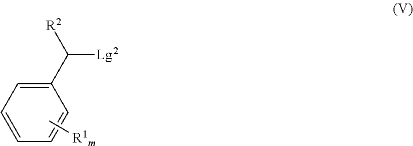

wherein the substituents are as defined above, and Lg² represents a suitable leaving group, such halogen (e.g. chloro, bromo, iodo); optionally in the presence of one or more reaction aids, such as a base (e.g. Na₂CO₃, or an inorganic salt (e.g. KI); optionally in the presence of one or more diluents, particularly polar solvents, (e.g. water, MeCN). This type of reaction is also known as alkylation reaction, typical reaction conditions are known in the field and may applied to the present process. Starting materials of formula (V) are known or obtainable according to known processes; starting materials of formula (IV) are obtainable according to the processes as described herein.

In one embodiment, the invention relates to a process for manufacturing a compound of formula (IV)

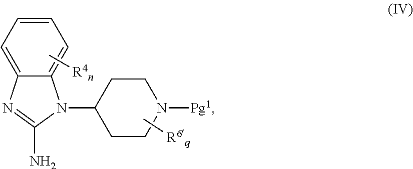

said method comprising the step of reacting a compound of formula (VI)

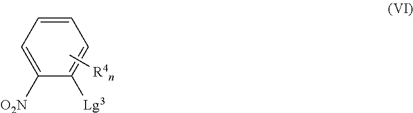

wherein the substituents are as defined above and Lg³ represents a suitable leaving group, particularly halo, e.g. fluoro with a compound of formula (VII),

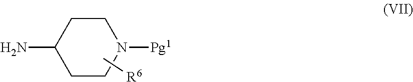

wherein the substituents are as defined above;

followed by the step of reacting the obtained intermediate with a reducing agent, such as hydrogen gas in the presence of a palladium (0) catalyst, or an organometallic salt, such as SnCl₂

followed by reacting the obtained intermediate with a compound of formula (VIII)

Lg⁴-CN (VIII)

wherein Lg⁴ represents a suitable leaving group, such as halogen, (e.g. bromo); optionally in the presence of one or more diluents, particular polar solvents (e.g. MeCN).

The above described first step is also known as aromatic nucleophilic substitution, the above described second step is known as reduction of a nitro to an amino group, the above described third step is known as a cyclisation reaction; typical reaction conditions for all steps are known in the field and may applied to the present process. Starting materials of formula (VI), (VII) and (VIII) are known or obtainable according to known processes.

In one embodiment, the invention relates to a process for manufacturing a compound of formula (IX)

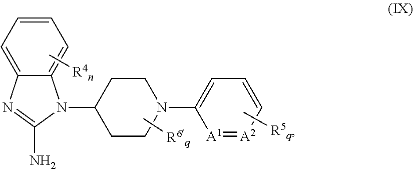

said method comprising the step of reacting a compound of formula (VI)

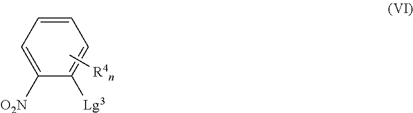

wherein the substituents are as defined above, and Lg³ represents a suitable leaving group, particularly halo (e.g. fluoro), with a compound of formula (X),

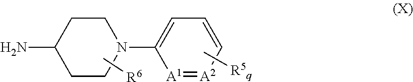

wherein the substituents are as defined above,

followed by the step of reacting the obtained intermediate with a reducing agent, such as hydrogen gas in the presence of a palladium (0) catalyst, or an organometallic salt, such as SnCl₂.

followed by reacting the obtained intermediate with a compound of formula (VIII)

Lg⁴-CN (VIII)

wherein Lg⁴ is as defined above; optionally in the presence of one or more diluents, particular polar solvents (e.g. MeCN).

The above described first step is also known as aromatic nucleophilic substitution, the above described second step is known as reduction of a nitro to an amino group, the above described third step is known as a cyclisation reaction; typical reaction conditions for all steps are known in the field and may applied to the present process. Starting materials of formula (VI) and (VIII) are known or obtainable according to known processes. Starting materials of formula (X) are obtainable according to the processes described herein.

In one embodiment, the invention relates to a process for manufacturing a compound of formula (X)

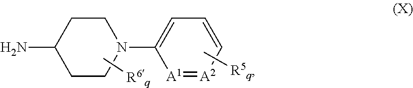

said method comprising the step of reacting a compound of formula (XI)

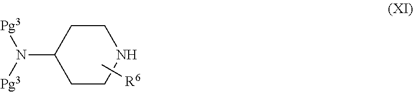

wherein the substituents are as defined above, and Pg³ represents a suitable protecting group (e.g. BOC), with a compound of formula (XII),

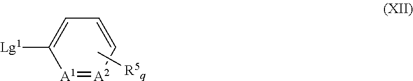

wherein the substituents are as defined above, optionally in the presence of one or more reaction aids, such as an organic or inorganic base (e.g. NEt₃, diisopropylethylamine, Na₂CO₃, Cs₂CO₃, K₂CO₃); optionally in the presence of one or more diluents, particularly one or more polar solvents (e.g. DMF, NMP, THF).

This type of reaction is also known as nucleophilic aromatic substitution, typical reaction conditions are known in the field and may applied to the present process. Starting materials of formula (XI) and (XII) are known or readily obtainable.

Further starting materials used in the above described processes are known, capable of being prepared according to known processes, or commercially obtainable; in particular, they can be prepared using processes as described in the Examples. In the preparation of starting materials, existing functional groups which do not participate in the reaction should, if necessary, be protected. Preferred protecting groups, their introduction and their removal are described above or in the examples. In place of the respective starting materials and transients, salts thereof may also be used for the reaction, provided that salt-forming groups are present and the reaction with a salt is also possible. Where the term starting materials is used hereinbefore and hereinafter, the salts thereof are always included, insofar as reasonable and possible.

Protecting Groups:

In the methods describe above, functional groups which are present in the starting materials and are not intended to take part in the reaction, are present in protected form if necessary, and protecting groups that are present are cleaved, whereby said starting compounds may also exist in the form of salts provided that a salt-forming group is present and a reaction in salt form is possible. In additional process steps, carried out as desired, functional groups of the starting compounds which should not take part in the reaction may be present in unprotected form or may be protected for example by one or more protecting groups. The protecting groups are then wholly or partly removed according to one of the known methods. Protecting groups, and the manner in which they are introduced and removed are described, for example, in “Protective Groups in Organic Chemistry”, Plenum Press, London, New York 1973, and in “Methoden der organischen Chemie”, Houben-Weyl, 4th edition, Vol. 15/1, Georg-Thieme-Verlag, Stuttgart 1974 and in Theodora W. Greene, “Protective Groups in Organic Synthesis”, John Wiley & Sons, New York 1981. A characteristic of protecting groups is that they can be removed readily, i.e. without the occurrence of undesired secondary reactions, for example by solvolysis, reduction, photolysis or alternatively under physiological conditions. Particularly, any amino group (—NH₂ or —NH) may be protected by a BOC group if the reaction takes place in basic conditions; such BOC group may be removed using a strong acid. Further, any amino group may be protected by an FMOC group if the reaction takes place in acidic conditions; such FMOC group may be removed using a strong acid.

Additional Process Steps:

In the methods described herein, (a) a compound of formula (I) obtained may be converted into another compound of formula (I), (b) a free compound of formula (I) may be converted into a salt, (c) a salt of a compound of formula (I) may be converted into the free compound or another salt, and/or (d) a mixture of isomeric compounds of formula (I) may separated into the individual isomers. Particularly, the conversion of R⁵ to another R⁵ (e.g. by reduction, substitution and/or oxidation) is considered such conversion (a) as described above. Further, the conversion of R³=hydrogen into another substituent R³ is considered such conversion (a).

General Process Conditions:

All process steps described here can be carried out under known reaction conditions, preferably under those specifically mentioned, in the absence of or usually in the presence of solvents or diluents, preferably those that are inert to the reagents used and able to dissolve them, in the absence or presence of catalysts, condensing agents or neutralising agents, for example ion exchangers, typically cation exchangers, for example in the H⁺form, depending on the type of reaction and/or reactants at reduced, normal, or elevated temperature, for example in the range from −100° C. to about 190° C., preferably from about −80° C. to about 150° C., for example at −80 to −60° C., at RT, at −20 to 40° C. or at the boiling point of the solvent used, under atmospheric pressure or in a closed vessel, if need be under pressure, and/or in an inert, for example an argon or nitrogen, atmosphere.

The invention relates also to those embodiments of the process in which one starts from a compound obtainable at any stage as an intermediate and carries out the missing steps, or breaks off the process at any stage, or forms a starting material under the reaction conditions, or uses said starting material in the form of a reactive derivative or salt, or produces a compound obtainable by means of the process according to the invention under those process conditions, and further processes the said compound in situ. In the preferred embodiment, one starts from those starting materials which lead to the compounds described hereinabove as preferred.

The compounds of formula (I) (or N-oxides thereof), including their salts, are also obtainable in the form of hydrates, or their crystals can include for example the solvent used for crystallisation (present as solvates).

In the preferred embodiment, a compound of formula (I) is prepared according to the processes and process steps defined in the Examples.

The invention relates in a third aspect to the use of compounds of the present invention as pharmaceuticals. Particularly, the compounds of formula (I) have valuable pharmacological properties, as described hereinbefore and hereinafter.

The invention thus also provides:
a compound of the formula (I) as defined herein, as pharmaceutical/for use as pharmaceutical;
a compound of the formula (I) as defined herein, as medicament/for use as medicament;
a compound of the formula (I) as defined herein, for the treatment of/for use in the treatment of an IGF-1R mediated disorders or diseases;
a compound of the formula (I) as defined herein, for the inhibition of the IGF-IR tyrosine kinase;
a compound of the formula (I) as defined herein, for the treatment of/for use in the treatment of a disorder or disease selected from multiple myeloma, neuroblastoma, synovial, hepatocellular, Ewing's Sarcoma, adrenocotical carcinoma (ACC) or a solid tumor selected from osteosarcoma, melanoma, tumor of breast, renal, prostate, colorectal, thyroid, ovarian, pancreatic, lung, uterine or gastrointestinal tumor;
the use of a compound of formula (I) as defined herein, for the treatment of/for the manufacture of a medicament for the treatment of an IGF-1R mediated disorder or disease;
the use of a compound of formula (I) as defined herein for the inhibition of the IGF-IR tyrosine kinase;
the use of a compound of formula (I) as defined herein, for the treatment of a disorder or disease selected from multiple myeloma, neuroblastoma, synovial, hepatocellular, Ewing's Sarcoma, adrenocotical carcinoma (ACC) or a solid tumor selected from osteosarcoma, melanoma, tumor of breast, renal, prostate, colorectal, thyroid, ovarian, pancreatic, lung, uterine or gastrointestinal tumor;
the use of a compound of formula (I) as defined herein, for the treatment of a disorder or disease selected from acute lung injury and pulmonary fibrosis;
a method of modulating IGF-1R activity in a subject, comprising the step of administering to a subject a therapeutically effective amount of a compound of formula (I) as definded herein;
a method for the treatment of an IGF-1R mediated disorder or disease comprising the step of administering to a subject a therapeutically effective amount of a compound of formula (I) as definded herein;
a method for inhibition IGF-1R in a cell, comprising contacting said cell with an effective amount of a compound of formula (I) as defined herein.

As used herein term “a therapeutically effective amount” of a compound of the present invention refers to an amount of the compound of formula (I) that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviating, inhibiting, preventing and/or ameliorating a condition, or a disorder or a disease (i) mediated by IGF-1R, or (ii) associated with IGF-1R activity, or (iii) characterized by activity (normal or abnormal) of IGF-1R; or (2) reducing or inhibiting the activity of IGF-1R; or (3) reducing or inhibiting the expression of IGF-1R. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of IGF-1R; or at least partially reducing or inhibiting the expression of IGF-1R. The meaning of the term “a therapeutically effective amount” as illustrated in the above embodiment for IGF-1R also applies by the same means to any other relevant proteins/peptides/enzymes. An “effective amount” may be determined empirically and in a routine manner, in relation to the stated purpose. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “administration” or “administering” of the subject compound means providing a compound of formula (I) and prodrugs thereof to a subject in need of treatment. Administration “in combination with” one or more further therapeutic agents includes simultaneous (concurrent) and consecutive administration in any order, and in any route of administration.

The term “cancer” refers to the physiological condition in mammals that is typically characterized by unregulated cell growth/proliferation. Examples of cancer include, but are not limited to: carcinoma, lymphoma, blastoma, and leukemia. More particular examples of cancers include, but are not limited to: chronic lymphocytic leukemia (CLL), lung, including non small cell (NSCLC), breast, ovarian, cervical, endometrial, prostate, colorectal, intestinal carcinoid, bladder, gastric, pancreatic, hepatic (hepatocellular), hepatoblastoma, esophageal, pulmonary adenocarcinoma, mesothelioma, synovial sarcoma, osteosarcoma, head and neck squamous cell carcinoma, juvenile nasopharyngeal angiofibromas, liposarcoma, thyroid, melanoma, basal cell carcinoma (BCC), adrenocotical carcinoma (ACC), medulloblastoma and desmoid.

As used herein, the term “IGF-1R mediated disease” includes but is not limited to, multiple myeloma, neuroblastoma, synovial, hepatocellular, Ewing's Sarcoma, adrenocotical carcinoma (ACC), or a solid tumor selected from osteosarcoma, melanoma, tumor of breast, renal, prostate, colorectal, thyroid, ovarian, pancreatic, lung, uterine or gastrointestinal tumor.

It was further found that compounds of formula (I) are also useful in the treatment of acute lung injury and pulmonary fibrosis.

The invention provides in further embodiments methods to treat, ameliorate or prevent a condition which responds to inhibition of IGF-1R in a mammal suffering from said condition, comprising administering to the mammal a therapeutically effective amount of a compound of formula (I) as defined herein, and optionally in combination with a second therapeutic agent. The compounds of the invention may be administered, for example, to a mammal suffering from an autoimmune disease, a transplantation disease, an infectious disease or a cell proliferative disorder. In particular examples, the compounds of the invention may be used alone or in combination with a chemotherapeutic agent to treat a cell proliferative disorder.

In a further embodiment, the invention relates to a process or a method for the treatment of one of the pathological conditions mentioned hereinabove, especially a disease which responds to an inhibition of the IGF-IR tyrosine kinase or of the IGF-IR-dependent cell proliferation, especially a corresponding neoplastic disease. The compounds of formula (I), or a pharmaceutically acceptable salt thereof, can be administered as such or in the form of pharmaceutical compositions, prophylactically or therapeutically, preferably in an amount effective against the said diseases, to a warm-blooded animal, for example a human, requiring such treatment, the compounds especially being used in the form of pharmaceutical compositions. In the case of an individual having a bodyweight of about 70 kg the daily dose administered is from approximately 0.1 g to approximately 5 g, preferably from approximately 0.5 g to approximately 2 g, of a compound of the present invention.

In a further embodiment, the invention relates to the use of a compound of formula (I), or a pharmaceutically acceptable salt thereof, especially a compound of formula (I) which is said to be preferred, or a pharmaceutically acceptable salt thereof, as such or in the form of a pharmaceutical composition with at least one pharmaceutically acceptable carrier, for the therapeutic and also prophylactic management of one or more of the diseases mentioned hereinabove, preferably a disease which responds to an inhibition of the IGF-IR tyrosine kinase or of the IGF-IR-dependent cell proliferation, especially a neoplastic disease, in particular if the said disease responds to an inhibition of the IGF-IR tyrosine kinase or of the IGF-IR-dependent cell proliferation.

In a further embodiment, the invention relates to the use of a compound of formula (I), or a pharmaceutically acceptable salt thereof, especially a compound of formula (I) which is said to be preferred, or a pharmaceutically acceptable salt thereof, for the preparation of a pharmaceutical composition for the therapeutic and also prophylactic management of one or more of the diseases mentioned hereinabove, especially a neoplastic disease, in particular if the disease responds to an inhibition of the IGF-IR tyrosine kinase or of the IGF-IR-dependent cell proliferation.

The invention relates in a fourth aspect to pharmaceutical compositions comprising a compound of the present invention.

The invention thus provides
a pharmaceutical composition comprising (i.e. containing or consisting of) a compound of formula (I) as defined herein and one or more carriers/excipients;
a pharmaceutical composition comprising a therapeutically effective amount of a compound of formula (I) as defined herein, and one or more pharmaceutically acceptable carriers/excipients.

As used herein, the term “pharmaceutically acceptable carrier” includes any and all solvents, dispersion media, coatings, surfactants, antioxidants, preservatives (e.g., anti-bacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

Suitable excipients/carriers may be any solid, liquid, semi-solid or, in the case of an aerosol composition, gaseous excipient that is generally available to one of skill in the art. Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols. Compressed gases may be used to disperse a compound of the formula (I) in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The dosage of the active ingredient depends upon the disease to be treated and upon the species, its age, weight, and individual condition, the individual pharmacokinetic data, and the mode of administration The amount of the compound in a formulation can vary within the full range employed by those skilled in the art. Typically, the formulation will contain, on a weight percent (wt %) basis, from about 0.01-99.99 wt % of a compound of formula (I) based on the total formulation, with the balance being one or more suitable pharmaceutical excipients. Preferably, the compound is present at a level of about 1-80 wt %. Unit dose forms are, for example, coated and uncoated tablets, ampoules, vials, suppositories or capsules. Examples are capsules containing from about 0.05 g to about 1.0 g of active substance.

Compositions for enteral administration, such as nasal, buccal, rectal or, especially, oral administration, and for parenteral administration, such as intravenous, intramuscular or subcutaneous administration, to warm-blooded animals, especially humans, are especially preferred. The compositions contain the compound of formula (I) alone or, preferably, together with a pharmaceutically acceptable carrier.

Pharmaceutical compositions comprising a compound of formula (I) as defined herein in association with at least one pharmaceutical acceptable carrier (such as an excipient and/or diluent) may be manufactured in conventional manner, e.g. by means of conventional mixing, granulating, coating, dissolving or lyophilising processes.

In a further embodiment, the invention relates to a pharmaceutical composition for administration to a warm-blooded animal, especially humans or commercially useful mammals suffering from a disease which responds to an inhibition of the IGF-IR tyrosine kinase or of the IGF-IR-dependent cell proliferation, comprising an effective quantity of a compound of formula (I) for the inhibition of the IGF-IR tyrosine kinase or of the IGF-IR-dependent cell proliferation, or a pharmaceutically acceptable salt thereof, together with at least one pharmaceutically acceptable carrier.

In a further embodiment, the invention relates to a pharmaceutical composition for the prophylactic or especially therapeutic management of neoplastic and other proliferative diseases of a warm-blooded animal, especially a human or a commercially useful mammal requiring such treatment, especially suffering from such a disease, comprising as active ingredient in a quantity that is prophylactically or especially therapeutically active against said diseases a new compound of formula (I), or a pharmaceutically acceptable salt thereof, is likewise preferred.

The invention relates in a fifth aspect to combinations comprising a compound of formula (I) and one or more additional active ingredients.

The invention thus provides
a combination in particular a pharmaceutical combination, comprising a therapeutically effective amount of a compound of formula (I) and one or more therapeutically active agents, particularly antiproliferative agents;
a combined pharmaceutical composition, adapted for simultaneous or sequential administration, comprising a therapeutically effective amount of a compound of formula (I) as defined herein; therapeutically effective amount(s) of one or more combination partners, particularly antiproliferative agents; one or more pharmaceutically acceptable excepients;
a combined pharmaceutical composition as defined herein (i) as pharmaceutical, (ii) for use in the treatment of a IGF-1R mediated disease, (iii) in a method of treatment of a IGF-1R mediated disease.

As used herein, the term “combination” refers to either a fixed combination in one dosage unit form, or a kit of parts for the combined administration where a compound of the formula (I) and a combination partner (e.g. an other drug as explained below, also referred to as “therapeutic agent” or “co-agent”) may be administered independently at the same time or separately within time intervals, especially where these time intervals allow that the combination partners show a cooperative, e.g. synergistic effect. The terms “coadministration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula (I) and a combination partner, are both administered to a patient simultaneously in the form of a single entity or dosage. The term “non-fixed combination” means that the active ingredients, e.g. a compound of formula (I) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The term “antiproliferative agent” includes, but are not limited to, aromatase inhibitors, antiestrogens, topoisomerase I inhibitors, topoisomerase II inhibitors, microtubule active agents, alkylating agents, histone deacetylase inhibitors, farnesyl transferase inhibitors, COX-2 inhibitors, MMP inhibitors, compounds decreasing the lipid kinase activity, eg PI3 kinase inhibitors, antineoplastic antimetabolites, platin compounds, compounds decreasing the protein kinase activity, eg mTOR inhibitors, Raf inhibitors, MEK inhibitors, and further anti-angiogenic compounds, gonadorelin agonists, anti-androgens, bengamides, bisphosphonates and trastuzumab, radiotherapy.

The term “aromatase inhibitors” as used herein relates to compounds which inhibit the estrogen production, i.e. the conversion of the substrates androstenedione and testosterone to estrone and estradiol, respectively. The term includes, but is not limited to steroids, especially exemestane and formestane and, in particular, non-steroids, especially aminoglutethimide, vorozole, fadrozole, anastrozole and, very especially, letrozole. Exemestane can be administered, e.g., in the form as it is marketed, e.g. under the trademark AROMASIN™. Formestane can be administered, e.g., in the form as it is marketed, e.g. under the trademark LENTARON™. Fadrozole can be administered, e.g., in the form as it is marketed, e.g. under the trademark AFEMA™. Anastrozole can be administered, e.g., in the form as it is marketed, e.g. under the trademark ARIMIDEXT™. Letrozole can be administered, e.g., in the form as it is marketed, e.g. under the trademark FEMARA™ or FEMAR™. Aminoglutethimide can be administered, e.g., in the form as it is marketed, e.g. under the trademark ORIMETEN™.

A combination of the invention comprising an aromatase inhibitor is particularly useful for the treatment of hormone receptor positive breast tumors.

The term “antiestrogens” as used herein relates to compounds which antagonize the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX™ Raloxifene hydrochloride can be administered, e.g., in the form as it is marketed, e.g. under the trademark EVISTA™. Fulvestrant can be formulated as disclosed in U.S. Pat. No. 4,659,516 or it can be administered, e.g., in the form as it is marketed, e.g. under the trademark FASLODEX™.

The term “topoisomerase I inhibitors” as used herein includes, but is not limited to topotecan, irinotecan, 9-nitrocamptothecin and the macromolecular camptothecin conjugate PNU-166148 (compound A1 in WO99/17804). Irinotecan can be administered, e.g., in the form as it is marketed, e.g. under the trademark CAMPTOSAR™. Topotecan can be administered, e.g., in the form as it is marketed, e.g. under the trademark HYCAMTIN™.

The term “topoisomerase II inhibitors” as used herein includes, but is not limited to the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide. Etoposide can be administered, e.g., in the form as it is marketed, e.g. under the trademark ETOPOPHOS™. Teniposide can be administered, e.g., in the form as it is marketed, e.g. under the trademark VM 26-BRISTOLT™. Doxorubicin can be administered, e.g., in the form as it is marketed, e.g. under the trademark ADRIBLASTIN™. Epirubicin can be administered, e.g., in the form as it is marketed, e.g. under the trademark FARMORUBICIN™. Idarubicin can be administered, e.g., in the form as it is marketed, e.g. under the trademark ZAVEDOS™. Mitoxantrone can be administered, e.g., in the form as it is marketed, e.g. under the trademark NOVANTRON™.

The term “lipid kinase inhibitors” relates to PI3 kinase inhibitors, PI4 kinase inhibitors, Vps34 inhibitors. Specific examples include: NVP-BEZ235, NVP-BGT226, NVP-BKM120, AS-604850, AS-041164, AS-252424, AS-605240, GDC0941, PI-103, TGX221, YM201636, ZSTK474, examples described in WO 2009/080705 and US 2009/163469.

The term “microtubule active agents” relates to microtubule stabilizing and microtubule destabilizing agents including, but not limited to the taxanes paclitaxel and docetaxel, the vinca alkaloids, e.g., vinblastine, especially vinblastine sulfate, vincristine especially vincristine sulfate, and vinorelbine, discodermolide and epothilones, such as epothilone B and D. Docetaxel can be administered, e.g., in the form as it is marketed, e.g. under the trademark TAXOTERE™. Vinblastine sulfate can be administered, e.g., in the form as it is marketed, e.g. under the trademark VINBLASTIN R.P.™. Vincristine sulfate can be administered, e.g., in the form as it is marketed, e.g. under the trademark FARMISTIN™. Discodermolide can be obtained, e.g., as disclosed in U.S. Pat. No. 5,010,099.

The term “alkylating agents” as used herein includes, but is not limited to cyclophosphamide, ifosfamide and melphalan. Cyclophosphamide can be administered, e.g., in the form as it is marketed, e.g. under the trademark CYCLOSTIN™. Ifosfamide can be administered, e.g., in the form as it is marketed, e.g. under the trademark HOLOXAN™

The term “histone deacetylase inhibitors” relates to compounds which inhibit the histone deacetylase and which possess antiproliferative activity.

The term “farnesyl transferase inhibitors” relates to compounds which inhibit the farnesyl transferase and which possess antiproliferative activity.

The term “COX-2 inhibitors” relates to compounds which inhibit the cyclooxygenase type 2 enyzme (COX-2) and which possess antiproliferative activity such as celecoxib (Celebrex®) and rofecoxib (Vioxx®).

The term “MMP inhibitors” relates to compounds which inhibit the matrix metalloproteinase (MMP) and which possess antiproliferative activity.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “antineoplastic antimetabolites” includes, but is not limited to 5-fluorouracil, 5-fluorouracil, tegafur, capecitabine, cladribine, cytarabine, fludarabine phosphate, fluorouridine, gemcitabine, 6-mercaptopurine, hydroxyurea, methotrexate, edatrexate and salts of such compounds, and furthermore ZD 1694 (RALTITREXED™), LY231514 (ALIMTA™), LY264618 (LOMOTREXOL™) and OGT719.

The term “platin compounds” as used herein includes, but is not limited to carboplatin, cis-platin and oxaliplatin. Carboplatin can be administered, e.g., in the form as it is marketed, e.g. under the trademark CARBOPLAT™. Oxaliplatin can be administered, e.g., in the form as it is marketed, e.g. under the trademark ELOXATIN™.

The term “compounds decreasing the protein kinase activity and further anti-angiogenic compounds” as used herein includes, but is not limited to compounds which decrease the activity of e.g. the Vascular Endothelial Growth Factor (VEGF), the Epidermal Growth Factor (EGF), and c-Src and anti-angiogenic compounds having another mechanism of action than decreasing the protein kinase activity.

Compounds which decrease the activity of VEGF are especially compounds which inhibit the VEGF receptor, especially the tyrosine kinase activity of the VEGF receptor, and compounds binding to VEGF, and are in particular those compounds, proteins and monoclonal antibodies generically and specifically disclosed in WO 98/35958 (describing compounds of formula (I)), WO 00/09495, WO 00/27820, WO 00/59509, WO 98/11223, WO 00/27819, WO 01/55114, WO 01/58899 and EP 0 769 947; those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, vol. 27, no. 1, pp 14-21, 1999; in WO 00/37502 and WO 94/10202; Angiostatin, described by M. S. O'Reilly et al, Cell 79, 1994, 315-328; and Endostatin, described by M. S. O'Reilly et al, Cell 88, 1997, 277-285; sorefanib (Nexavar), Sutent (sunitinib), BAY 43-9006.

Compounds which decrease the activity of EGF are especially compounds which inhibit the EGF receptors, especially the tyrosine kinase activity of the EGF receptors, and compounds binding to EGF, and are in particular those compounds generically and specifically disclosed in WO 97/02266 (describing compounds of formula (I)V), EP 0 564 409, WO 99/03854, EP 0520722, EP 0 566 226, EP 0 787 722, EP 0 837 063, WO 98/10767, WO 97/30034, WO 97/49688, WO 97/38983 and, especially, WO 96/33980. Specific EGF receptor inhibitor examples include, but not limited to; Tarceva (erlotinib), Iressa (Gefitinib), Tywerb (lapatanib). Erbitux (cetuximab), Avastin (bevacizumab), Herceptin (trastuzamab), Rituxan (rituximab), Bexxar (tositumomab), panitumumab.

Compounds which decrease the activity of c-Src include, but are not limited to, compounds inhibiting the c-Src protein tyrosine kinase activity as defined below and to SH2 interaction inhibitors such as those disclosed in WO97/07131 and WO97/08193; compounds inhibiting the c-Src protein tyrosine kinase activity include, but are not limited to, compounds belonging to the structure classes of pyrrolopyrimidines, especially pyrrolo[2,3-d]pyrimidines, purines, pyrazopyrimidines, especially pyrazo[3,4-d]pyrimidines, pyrazopyrimidines, especially pyrazo[3,4-d]pyrimidines and pyridopyrimidines, especially pyrido[2,3-d]pyrimidines. Preferably, the term relates to those compounds disclosed in WO 96/10028, WO 97/28161, WO97/32879 and WO97/49706;

Compounds which decrease the activity of Raf kinases include, but are not limited to: Raf265, sorefanib, BAY 43-9006.

Compounds which inhibit downstream effectors of Raf kinases, such as MEK. Examples of MEK inhibitors include; PD 98059, AZD6244 (ARRY-886), CI-1040, PD 0325901, u0126.

Anti-angiogenic compounds having another mechanism of action than decreasing the protein kinase activity include, but are not limited to e.g. thalidomide (THALOMID™), SU5416, and celecoxib (Celebrex™).

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX™.

The term “anti-androgens” as used herein includes, but is not limited to bicalutamide (CASODEX™), which can be formulated, e.g. as disclosed in U.S. Pat. No. 4,636,505.

The term “bengamides” relates to bengamides and derivatives thereof having aniproliferative properties and includes, but is not limited to the compounds generically and specifically disclosed in WO00/29382, preferably to ex. 1 of WO00/29382.

The term “bisphosphonates” as used herein includes, but is not limited to etridonic acid, clodronic acid, tiludronic acid, pamidronic acid, alendronic acid, ibandronic acid, risedronic acid and zoledronic acid. “Etridonic acid” can be administered, e.g., in the form as it is marketed, e.g. under the trademark DIDRONEL™. “Clodronic acid” can be administered, e.g., in the form as it is marketed, e.g. under the trademark BONEFOS™ “Tiludronic acid” can be administered, e.g., in the form as it is marketed, e.g. under the trademark SKELID™. “Pamidronic acid” can be administered, e.g., in the form as it is marketed, e.g. under the trademark AREDIA™. “Alendronic acid” can be administered, e.g., in the form as it is marketed, e.g. under the trademark FOSAMAX™. “Ibandronic acid” can be administered, e.g., in the form as it is marketed, e.g. under the trademark BONDRANAT™. “Risedronic acid” can be administered, e.g., in the form as it is marketed, e.g. under the trademark ACTONEL™. “Zoledronic acid” can be administered, e.g., in the form as it is marketed, e.g. under the trademark ZOMETAT™.

“Trastuzumab” can be administered, e.g., in the form as it is marketed, e.g. under the trademark HERCEPTIN™.

The structure of the active agents identified by code nos., generic or trade names may be taken from the actual edition of the standard compendium “The Merck Index” or from databases, e.g. Patents International (e.g. IMS World Publications). The above-mentioned compounds, which can be used in combination with a compound of formula (I), can be prepared and administered as described in the art such as in the documents cited above.

The following Examples serve to illustrate the invention without limiting its scope. Abbreviations used are those conventional in the art or the following:

| | |
|---|---|
| AcOEt | Ethyl acetate |
| amu | Atomic mass units |
| Boc | Tert-Butoxycarbonyl |
| Boc₂O | Di-tert-butyl dicarbonate |
| CH2Cl2 | dichloromethane |
| DCE | 1,2-dichloroethane |
| DIBAL-H | Diisobutylaluminium hydride |
| DIC | Diisopropyl carbodiimide |
| DIPEA | Diisopropylethylamine |
| DMAP | N,N-Dimethylpyridin-4-amin |
| DME | 1,2-Dimethoxyethane |
| DMF | N,N-Dimethylformamide |
| dppf | 1,1′-bis(diphenylphosphanyl) ferrocene |
| HATU | (2-(7-Aza-1H-benzotriazole-1- |
| | yl)-1,1,3,3-tetramethyluronium- |
| | hexafluorophosphate) |
| LiAlH4 | Lithium-aluminium hydride |
| mCPBA | m-chloroperbenzoic acid |
| MeCN | Acetonitrile |
| MeOH | Methanol |
| Na2SO4 | Sodium sulfate |
| NaOH | Sodium hydroxyde |
| NEt₃ | Triethylamine |
| NH4OH | Aqueous ammonia solution, 30% |
| NMP | N-methylpyrrolidinone |
| Pd(Ph3P) 4 | Tetra-kis(triphenylphosphane)palladium(0) |
| Pd/C | Palladium on carbon |
| PTFE | Polytetrafluorethylene, Teflon |
| Rac- | rac-2,2′-bis(diphenylphosphino)-1,1′- |
| BINAP | binaphthyl |
| rt | room temperature |
| TEA | triethylamine |
| TFA | Trifluoroacetic acid |
| THF | Tetrahydrofuran |
| t_{R} | Retention time |

### I Analytical Methods

Temperatures are measured in degrees Celsius.

Nuclear magnetic resonance spectra were recorded on a Bruker spectrometer at 400 mHz and at room temperature.

The following HPLC, MS and HPLC/MS methods are used in the preparation of the Intermediates and Examples:

HPLC/MS Method A

Instrument: Waters Acquity Ultra Performance LC system, Waters 2996 photodiode array UV detector, Water SQ MS detector (range: 130-750 amu; cone: +10V and −30V), column oven temperature +40° C.

Column: Acquity UPLC BRH C18 1.7 um, 2.1*50 mm

Flow rate: 0.7 mL/min

Eluent: A: water+0.1% formic acid; B: acetonitrile+0.1% formic acid

Gradient:

| Time (min) | % B in A |
|---|---|
| 0 | 5 |
| 2 | 100 |

HPLC/MS Method B

Instrument: Agilent 1100 LC chromatography system with Micromass ZMD MS detection (range: 100-900 amu; cone: +25V), column oven temperature +50° C.

Column: Ascentis Express C18 2.1 mm×30 mm 2.7 u particles

Eluent: A: water+0.1% TFA; B: acetonitrile+0.1% TFA

Gradient:

| Time (min) | Flow rate (μL/min) | % B in A |
|---|---|---|
| 0 | 1200 | 10 |
| 1.7 | 1400 | 95 |
| 2.4 | 2400 | 95 |
| 2.45 | 2400 | 10 |
| 2.5 | 1200 | 10 |

HPLC/MS Method D

Instrument: Waters Acquity Ultra Performance LC system, Waters 2996 photodiode array

UV detector, Water SQ MS detector (range: 130-750 amu; cone: +10V and −30V), column oven temperature +40° C.

Column: Acquity UPLC BRH C18 1.7 um, 2.1*50 mm

Flow rate: 0.7 mL/min

Eluent: A: water+0.1% formic acid; B: acetonitrile+0.1% formic acid

Gradient:

| Time (min) | % B in A |
|---|---|
| 0 | 20 |
| 1 | 25 |
| 4.2 | 90 |
| 4.3 | 100 |

HPLC Method E

Instrument: Waters HPLC system, Water 2545 binary gradient module, Waters 2996 photodiode array UV detector, Waters 2767 Auto Sampler/Fraction Collector.

Column: sunfire Prep C18 OBD 5 um, 30*100 mm.

Flow rate: 30 mL/min

Generic eluents Eluent: A: water+0.1% TFA; B: acetonitrile+0.1% TFA

Generic gradient from 0% B in A to 100% B in A over 20 minutes.

The generic conditions were used unless specified in the experimental part for individual intermediates/examples.

### II Chemical Synthesis

### Intermediates

### Intermediate N: 4-Bromomethyl-indole-1-carboxylic acid tert-butyl ester

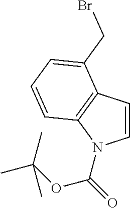 [SMILES: CC(C)(C)OC(=O)n1ccc2c(CBr)cccc21]

To a solution of 4-Hydroxymethyl-indole-1-carboxylic acid tert-butyl ester (step N.1, 17.0 g, 68.7 mmol) in CH2Cl2 (229 ml) was added tetrabromomethane (25.1 g, 76 mmol) and the yellow clear solution cooled to 0° C. Then triphenylphosphine (27.0 g, 103 mmol) was added in portions over the course of 10 minutes. The resulting mixture was stirred at 0° C. for 1 hr. The mixture was then treated with water (150 mL) and the medium was vigorously stirred for an hour at rt. The two phases were separated, the aqueous layer was extracted with CH₂Cl₂ (250 mL), the combined organic layers were dried over Na2SO4, filtered and concentrated to a clear brown oil. The crude product was purified by chromatography on silica gel, eluting with CH₂Cl₂/heptane:1/1, leading to the title compound as a clear oil, 16.2 g (76%). HPLC/MS (method A) t_{R}1.88 minute, M-Br 230. 1H NMR (Dimethylsulfoxyde-d6) Ppm 1.64 (s, 9H) 5.00 (s, 2H) 6.92 (d, 1H) 7.25-7.38 (m, 2H) 7.77 (d, 1H) 8.04 (d, 1H).

### Step N.1: 4-Hydroxymethyl-indole-1-carboxylic acid tert-butyl ester

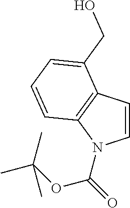 [SMILES: CC(C)(C)OC(=O)n1ccc2c(CO)cccc21]

To a solution of indole-1,4-dicarboxylic acid 1-tert-butyl ester 4-methyl ester (step N.2, 20.0 g, 72.6 mmol) in THF (363 ml) at −78° C. under Ar was added DIBAL-H 1M in cyclohexane (145.0 ml, 145 mmol) dropwise. The resulting solution was stirred at −78° C. for 20 minutes, allowed to reach rt slowly and then stirred at rt temperature for 16 hours. The reaction mixture was then cooled to 0° C. and Rochelle's salt solution (200 mL) was carefully added (strong exotherm). The resulting mixture was stirred for 2 hours. The phases were separated and the organic layer was concentrated to remove the maximum of the solvent. The resulting solution was diluted with AcOEt (400 mL) and the organic phase was then washed with Rochelle's salt solution (2*200 mL), water (1×200 mL) and brine (1×200 mL). The organic layer was then dried over Na2SO4, filtered and evaporated to dryness to give a clear brown oil, 18.1 g (96%). HPLC/MS (method A) tR1.38 minute. 1H NMR (Dimethylsulfoxyde-d6) Ppm 1.63 (s, 9H) 4.75 (d, 2H) 5.24 (t, 1H) 6.81 (d, 1H) 7.18-7.36 (m, 2H) 7.66 (d, 1H) 7.96 (d, 1H).

### Step N.2: Indole-1,4-dicarboxylic acid 1-tert-butyl ester 4-methyl ester

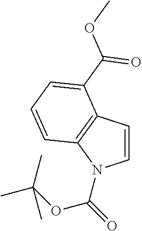 [SMILES: COC(=O)c1cccc2c1ccn2C(=O)OC(C)(C)C]

To a solution of methyl Indole-4-carboxylate (5 g, 28.5 mmol) in MeCN (40.8 ml) was added DMAP (0.174 g, 1.427 mmol) followed by Boc2O (7.95 ml, 34.2 mmol). The resulting solution was stirred for 16 hours. The reaction mixture was then evaporated to dryness. The crude was diluted in EtOAc (200 mL) and washed successively with 10% aqueous citric acid solution (3×100 mL), saturated aqueous NaHCO3 solution (2×100 mL), and saturated aqueous NaCl solution (1×100 mL). The organic layer was dried over Na2SO4, filtered and concentrated to give the title compound as a clear oil, 7.86 g (100%), no further purification carried out. HPLC/MS (method A) t_{R}1.41 minute, M+H 276. 1H NMR (Dimethylsulfoxyde-d6) Ppm 1.64 (s, 9H) 3.92 (s, 3H) 7.20 (d, 1H) 7.44 (t, 1H) 7.84 (d, 1H) 7.90 (dd, 1H) 8.36 (d, 1H).

### Intermediate AG: 4-Bromomethyl-benzoimidazole-1-carboxylic acid tert-butyl ester

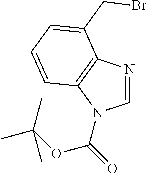 [SMILES: CC(C)(C)OC(=O)n1cnc2c(CBr)cccc21]

To a solution of 4-Hydroxymethyl-benzoimidazole-1-carboxylic acid tert-butyl ester (Step AG1, 330 mg, 1.329 mmol) in Et2O (5 ml) at 0° C. under Ar was added PBr3 (1M in CH₂Cl₂) (1.462 ml, 1.462 mmol), dropwise, causing a thick precipitation. After 5 minutes at 0° C., the medium was allowed to reach rt. After 1 h at rt, PBr3 (1M in CH₂Cl₂) (0.133 ml, 0.133 mmol) was added under stirring, followed by CH₂Cl₂ (2 ml). After a further 1 h at rt, the medium was carefully quenched with saturated sodium bicarbonate solution (20 ml, strong CO2 evolution) and extracted with EtOAc (3×15 ml). Drying of the combined organics over Na2SO4 and concentration afforded a clear oil. The oil was purified by chromatography on silica gel, using a 0% to 100% gradient of eluent B (EtOAc/CH₂Cl₂/heptane: 1/2/2) in eluent A (heptane/CH2Cl2:1/1), yielding the title product as a colorless oil, 153 mg (37%). HPLC/MS (Method A) t_{R}1.70 minute, M+H 310.9-312.9. 1H NMR (DMSO-d₆) Ppm 1.69 (s, 9H) 4.95 (s, 2H) 7.38 (m, 2H) 7.94 (d, 1H) 8.49 (s, 1H).

### Step AG1: 4-Hydroxymethyl-benzoimidazole-1-carboxylic acid tert-butyl ester

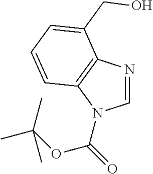 [SMILES: CC(C)(C)OC(=O)n1cnc2c(CO)cccc21]

To a solution of (1H-Benzoimidazol-4-yl)-methanol (Step AG2, 600 mg, 4.05 mmol) in MeCN (32 mL) and Water (8 mL) was added sodium bicarbonate (680 mg, 8.10 mmol) followed by BOC₂O (1.081 mL, 4.66 mmol). The resulting mixture was stirred at rt for 4 hrs before careful evaporation to dryness. The crude was taken up in AcOEt (200 mL) and extracted with 10% aqueous citric acid solution (3×100 mL), water (2×100 mL) and brine (100 mL). The organic layer was dried over Na2SO4, filtered and concentrated to give an oily white solid (855 mg, 85%). No purification was required. HPLC/MS (Method A) t_{R}1.16 minute, M+H 249.0. 1H NMR (DMSO-d₆) Ppm 1.66 (s, 9H) 4.93 (d, 2H) 5.26 (t, 1H) 7.36-7.47 (m, 2H) 7.82 (dd, 1H) 8.61 (s, 1H).

### Step AG2: (1H-Benzoimidazol-4-yl)-methanol

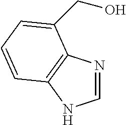 [SMILES: OCc1cccc2[nH]cnc12]

To a solution of 1H-Benzoimidazole-4-carboxylic acid methyl ester (Step AG3, 1.0 g, 5.68 mmol) in THF (56.8 mL) under argon was added LiAlH4 (1M in THF) (6.24 mL, 6.24 mmol), dropwise, causing a yellow coloration and a slight gas evolution. The reaction mixture was stirred at rt for 75 min. The medium was carefully quenched by addition of saturated aqueous NH₄Cl solution (50 mL). The slurry of aluminium salts was stirred for an hour at rt. The organic supernatant was decanted and the insoluble aluminium salts suspension was extracted with AcOEt (3×100 mL). The combined organic layers were dried over Na2SO4, filtered and concentrated under reduced pressure to give a colorless oil (600 mg, 71%). HPLC/MS (Method A) t_{R}0.64 minute, M+H 149.0. 1H NMR (DMSO-d₆) Ppm 4.85 (br. s., 2H) 5.19 (br. s., 1H) 7.07-7.26 (m, 2H) 7.48 (d, J=7.34 Hz, 1H) 8.19 (s, 1H) 12.35-12.64 (m, 1H).

### Step AG3: 1H-Benzoimidazole-4-carboxylic acid methyl ester

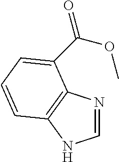

To a solution of 1H-benzimidazole-7-carboxylic acid (1 g, 6.17 mmol) in MeOH (20.56 mL) at 0° C. was added DMAP (0.151 g, 1.233 mmol) followed by DIC (1.057 mL, 6.78 mmol). The resulting reaction mixture was warmed up to rt and was stirred at rt for 4 hrs. The reaction mixture was then concentrated to dryness under vacuum. The crude was taken up in AcOEt (200 mL) and washed with saturated aqueous sodium bicarbonate solution (3×100 mL) and brine (100 mL). The organic layer was dried over Na2SO4, filtered and evaporated to give a clear oil. The residue was purified by chromatography on silica gel, using a 5% to 100% gradient of eluent B (EtOAc/MeOH/NH4OH:90/9/1) in eluent A (heptane/CH2Cl2:1/1), yielding the title product as a white solid, 550 mg (51%). HPLC/MS (Method A) t_{R}0.70 minute, M+H 177.0. 1H NMR (DMSO-d₆) Ppm 3.95 (s, 3H) 7.32 (t, 1H) 7.86 (d, 1H) 7.97 (d, 1H) 8.31 (s, 1H) 12.57 (br. s., 1H)

### Intermediate AH: 4-Bromomethyl-benzotriazole-1-carboxylic acid tert-butyl ester

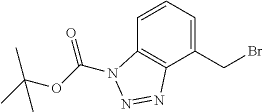 [SMILES: CC(C)(C)OC(=O)n1nnc2c(CBr)cccc21]

To a solution of 4-hydroxymethyl-benzotriazole-1-carboxylic acid tert-butyl ester (Step AH1, 1.58 g, 6.34 mmol) in CH₂Cl₂ (24 mL) was added triphenylphosphine (2.494 g, 9.51 mmol). The mixture was chilled to 0° C. then a solution of CBr4 (3.15 g, 9.51 mmol) in CH₂Cl₂ (24.00 mL) was added dropwise and stirring was maintained at 0° C. for 1.5 hrs. The reaction mixture was then evaporated to a crude residue which was purified by chromatography on silica gel, using a 3% to 15% gradient of eluent B (EtOAc) in eluent A (cyclohexane), yielding the title product as a clear oil, 1.59 g (80%). HPLC/MS (Method D) t_{R}2.83 minute, M+H 312.314. 1H NMR (DMSO-d₆) Ppm 1.71 (s, 9H) 5.18 (s, 2H) 7.66 (d, 1H) 7.75 (t, 1H) 7.99 (d, 1H)

### Step AH1: 4-Hydroxymethyl-benzotriazole-1-carboxylic acid tert-butyl ester

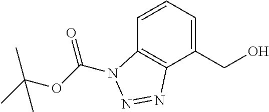 [SMILES: CC(C)(C)OC(=O)n1nnc2c(CO)cccc21]

To a well stirred suspension of (1H-Benzotriazol-4-yl)-methanol (1.9 g, 12.74 mmol, synthesized as described in: Hurt, Clarence Ray; Pennell, Andrew. K.; Wright, John Jessen; Wang, Qiang; Leleti, Manmohan; Reddy; Thomas, William D.; Li, Yandong; Dragoli, Dean R. Substituted dihydropyridines as C5a receptor modulators and their preparation, pharmaceutical compositions and use in the treatment of diseases. WO2007051062) and sodium bicarbonate (2.140 g, 25.5 mmol) in MeCN (18 mL) and water (12 mL) was added a solution of Boc2O (3.40 mL, 14.65 mmol) in MeCN (18 mL). Stirring was kept at RT for 2 hr. The reaction mixture was poured onto 30 ml of aqueous 0.5 M citric acid solution then extracted with EtOAc (3×50 mL). The combined organic layers were washed with brine then dried over MgSO4. The residue was purified by chromatography on silica gel, using a 10% to 40% gradient of eluent B (EtOAc) in eluent A (cyclohexane), yielding the title product as a clear oil, 2.97 g (94%). HPLC/MS (Method D) t_{R}1.81 minute, M+H 250.0. 1H NMR (DMSO-d₆) Ppm 1.71 (s, 9H) 5.10 (d, 2H) 5.54 (t, 1H) 7.58 (d, 1H) 7.73 (t, 1H) 7.89 (d, 1H)

### Intermediate AI: 4-(2-Amino-benzoimidazol-1-yl)-piperidine-1-carboxylic acid tert-butyl ester

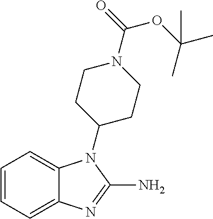 [SMILES: CC(C)(C)OC(=O)N1CCC(n2c(N)nc3ccccc32)CC1]

4-(2-Amino-phenylamino)-piperidine-1-carboxylic acid tert-butyl ester (Step AI1, 4.84 g, 16.6 mmol) was dissolved in MeCN (156 mL) and water (10.4 mL). To this dark solution was added cyanogen bromide (5M solution in MeCN, 3.49 mL, 17.5 mmol) and the resulting reaction mixture was stirred at rt for 22 hours. The reaction mixture was concentrated under vacuum, diluted with EtOAc (100 mL) and washed successively with saturated aqueous sodium bicarbonate solution (3×100 mL) and brine (1×100 mL). The organic phase was dried over Na₂SO₄, filtered and evaporated to drynessto afford the title compound without the need for further purification, 5.53 g (quantitative). HPLC/MS (Method A) t_{R}1.01 minute, M+H 317.1. 1H NMR (DMSO-d₆) Ppm 1.46 (s, 9H) 1.74 (bd, 2H) 2.18 (qd, 2H) 2.86 (bs, 2H) 4.13 (bd, 2H) 4.38 (m, 1H) 6.31 (s, 2H) 6.83 (t, 1H) 6.92 (t, 1H) 7.13 (d, 1H) 7.19 (d, 1H)

### Step AI1: 4-(2-Amino-phenylamino)-piperidine-1-carboxylic acid tert-butyl ester

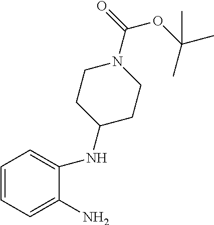 [SMILES: CC(C)(C)OC(=O)N1CCC(Nc2ccccc2N)CC1]

4-(2-Nitro-phenylamino)-piperidine-1-carboxylic acid tert-butyl ester (Step AI2, 5.50 g, 17.1 mmol) was dissolved in MeOH (172 mL) and Pd/C (10% w/w) (0.91 g, 0.86 mmol) was added. The flask was then purged several times with hydrogen gas and finally placed under one atmosphere of hydrogen gas. The medium was then stirred at rt for 2 hours. The reaction mixture was filtered through a pad of celite which was rinsed with MeOH (2×20 mL). The combined filtrates were concentrated and filtered again through a 0.2 uM PTFE membrane. Final filtrate was then evaporated to dryness and dried under high vacuum to afford the title compound, 4.83 g (97%). HPLC/MS (Method A) t_{R}1.05 minute, M+H 292.1.

### Step AI2: 4-(2-Nitro-phenylamino)-piperidine-1-carboxylic acid tert-butyl ester

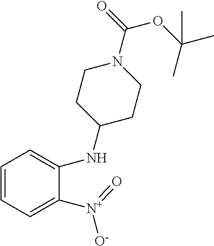 [SMILES: CC(C)(C)OC(=O)N1CCC(Nc2ccccc2[N+](=O)[O-])CC1]

To a solution of 4-amino-1-Bocpiperidine (5.00 g, 24.96 mmol) in DMF (42 mL) were added DIPEA (5.45 mL, 31.2 mmol) and 2-fluoro-1-nitro-benzene (2.20 mL, 20.80 mmol). The resulting reaction mixture was stirred at 80° C. After 16 hours, the reaction mixture was poured into a 10% aqueous citric acid solution (300 mL). The resulting biphasic mixture was vigorously stirred for a few minutes. The mixture was then extracted with EtOAc (3×100 mL). The combined organic layers were then back-extracted with 10% aqueous citric acid solution (2×100 mL) and brine (1×100 mL). The organic phase was then dried over Na₂SO₄, filtered and evaporated to dryness. The residue was purified by chromatography on silica gel, using a 5% to 100% gradient of eluent B (EtOAc) in eluent A (heptane), yielding the title product as a yellow solid, 5.50 g (83%). HPLC/MS (Method A) t_{R}1.69 minute, M+Na 344.1.

### Intermediate AJ: 4-(2-Amino-6-fluoro-benzoimidazol-1-yl)-piperidine-1-carboxylic acid tert-butyl ester

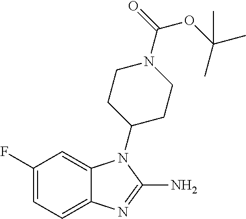 [SMILES: CC(C)(C)OC(=O)N1CCC(n2c(N)nc3ccc(F)cc32)CC1]

The title compound was synthesized in a manner analogous to that used for the synthesis of intermediate AI, using 2,4-difluoro-1-nitro-benzene instead of 2-fluoro-1-nitrobenzene. HPLC/MS (Method A) t_{R}0.98 minute, M+H 335.1. 1H NMR (DMSO-d₆) Ppm 1.46 (s, 9H) 1.72 (bd, 2H) 2.15 (m, 2H) 2.86 (bs, 2H) 4.13 (bd, 2H) 4.37 (m, 1H) 6.38 (s, 2H) 6.75 (td, 1H) 7.05 (m, 2H).

### Intermediate AN: 1-(2-Chloro-benzyl)-5-fluoro-3-piperidin-4-yl-1,3-dihydro-benzoimidazol-2-ylideneamine

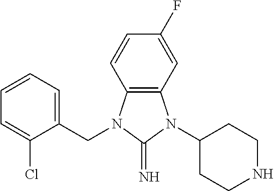 [SMILES: N=c1n(Cc2ccccc2Cl)c2ccc(F)cc2n1C1CCNCC1]

4-[3-(2-Chloro-benzyl)-6-fluoro-2-imino-2,3-dihydro-benzoimidazol-1-yl]-piperidine-1-carboxylic acid tert-butyl ester (Step AN1, 618 mg, 1.35 mmol) was dissolved in CH₂Cl₂ (7 mL) and a solution of TFA (3.11 mL, 40 mmol) and water (0.06 mL) was added. The mixture was stirred at rt for 1 h. All volatiles were removed under vacuum and the oily residue was azeotroped with toluene (3×30 mL) to give a dark brown oily substance. The crude material was dissolved in a mixture of CH₂Cl₂ (30 mL) and methanol (2 mL). To this was added polymer bound tetraalkylammonium carbonate (1.8 g pre-washed with CH₂Cl₂ and methanol) and the mixture was gently shaken for 20 min. The resin was removed by filtration, washed with CH₂Cl₂ (20 mL), and the filtrate was evaporated to give the title compound as a waxy green solid that was used in subsequent steps without any purification, 523 mg (97%) M+H 359.5, 361.5 1H NMR (Methanol-d4) Ppm 1.99-2.06 (m, 2H) 2.45-2.56 (m, 2H) 2.86-2.94 (m, 2H) 3.34-3.40 (m, 2H) 4.61-4.70 (m, 1H) 5.45 (s, 2H) 6.95 (d, 1H) 7.00 (dt, 1H) 7.12-7.16 (m, 1H) 7.26-7.30 (m, 1H) 7.33-7.38 (m, 1H) 7.53 (dd, 1H) 7.66 (dd, 1H)

### Step AN1: 4-[3-(2-Chloro-benzyl)-6-fluoro-2-imino-2,3-dihydro-benzoimidazol-1-yl]-piperidine-1-carboxylic acid tert-butyl ester

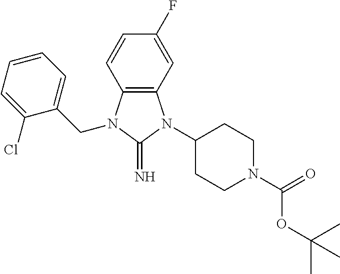 [SMILES: CC(C)(C)OC(=O)N1CCC(n2c(=N)n(Cc3ccccc3Cl)c3ccc(F)cc32)CC1]

4-(2-Amino-6-fluoro-benzoimidazol-1-yl)piperidine-1-carboxylic acid tert-butyl ester (Intermediate AJ, 600 mg, 1.79 mmol) was dissolved in MeCN (20 mL). Potassium iodide (298 mg, 1.79 mmol) and 2-chlorobenzyl bromide (0.23 mL, 1.79 mmol) were added and the mixture was stirred at rt for 30 min. The volatile components were evaporated and the crude residue was dissolved in DMF (10 mL). To this was added water (50 mL) and the resulting precipitate was collected by filtration and washed with water (50 mL). The crude solid product was purified by chromatography on silica gel, using a 0% to 100% gradient of eluent B (CH₂Cl₂/MeOH:8/2) in eluent A (CH₂Cl₂), yielding the title product, 88 mg, and unreacted starting material, 413 mg. The recovered starting material was reacted with 1 eq. 2-chlorobenzyl bromide in MeCN at 110° C. for 30 min. An additional 0.3 eq. of 2-chlorobenzyl bromide was added and the mixture was stirred at rt for 48 h. The product was purified by chromatography on silica gel, using a 0% to 100% gradient of eluent B (CH₂Cl₂/MeOH:8/2) in eluent A (CH₂Cl₂), yielding the title product, 526 mg, for a combined yield of 614 mg (71%).

1H NMR (Methanol-d4) Ppm 1.52 (s, 9H) 2.04 (d, 2H) 2.34-2.46 (m, 2H) 2.95-3.07 (m, 2H) 4.67 (d, br, 2H) 4.60-4.70 (m, 1H) 5.50 (s, 2H) 6.98 (dd, 1H) 7.05 (dt, 1H) 7.20 (dd, 1H) 7.29 (dt, 1H) 7.37 (dt, 1H) 7.52-7.56 (m, 2H)

### III Chemical Synthesis

### Compounds According to the Invention

### Example 54

### 1-(2-Chloro-benzyl)-3-(1-m-tolyl-piperidin-4-yl)-1,3-dihydro-benzoimidazol-2-ylideneamine

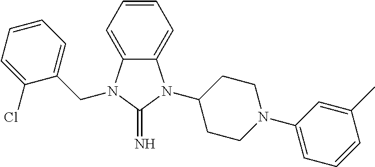 [SMILES: Cc1cccc(N2CCC(n3c(=N)n(Cc4ccccc4Cl)c4ccccc43)CC2)c1]

To a suspension of 1-(1-m-Tolyl-piperidin-4-yl)-1H-benzoimidazol-2-ylamine (step 1, 50 mg, 0.163 mmol) in MeCN (2.0 mL) were added 2-chlorobenzylbromide (21.3 μL, 0.163 mmol) and KI (27.6 mg, 0.163 mmol). The resulting mixture was heated to 110° C. for 10 minutes under microwave irradiation. The medium was then evaporated to dryness and the residue partitionned between water (6 mL) and EtOAc (5 mL). The organic phase was separated and the aqueous layer was further extracted with EtOAc (2×5 mL). The combined organic extracts were then dried over Na2SO4, filtered and evaporated to a thick residue. The crude product was purified by reverse-phase preparative HPLC (Method E, gradient from 20% (MeCN +0.1% TFA) in (Water +0.1% TFA) to 80% over 14 minutes). Product-containing fractions were pooled and evaporated to dryness to furnish the title product as its TFA salt, 15 mg (16%). HPLC/MS (Method A) t_{R}1.34 minute (>95% UV purity), [(M+2H)/2] 216.1.

### Step 1: 1-(1-m-Tolyl-piperidin-4-yl)-1H-benzoimidazol-2-ylamine

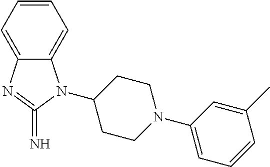

A mixture of N-(1-m-Tolyl-piperidin-4-yl)-benzene-1,2-diamine (step 2, 603 mg, 2.14 mmol) and cyanic bromide (990 mg, 9.16 mmol) dissolved in MeCN (10.7 mL) and water (0.7 mL) was stirred for one hour at rt. The reaction mixture was then evaporated to dryness. The residue was partitionned between CH₂Cl₂ (10 mL) and 1N aqueous sodium hydroxide solution (10 mL). The phases were separated and the aqueous layer was further extracted with CH₂Cl₂ (2×10 mL). The combined organic phases were dried over Na2SO4, filtered and evaporated to dryness to afford the title product as a yellow solid, 650 mg (99%). HPLC/MS (Method A) t_{R}1.02 minute, M+H 307.0. 1H NMR (DMSO-d₆) Ppm 1.81 (d, 2H) 2.28 (s, 3H) 2.45 (qd, 2H) 2.83 (t, 2H) 3.87 (d, 2H) 4.39 (m, 1H) 6.35 (bs, 2H) 6.63 (d, 1H) 6.80-6.95 (m, 4H) 7.14 (m, 3H).

### Step 2: N-(1-m-Tolyl-piperidin-4-yl)-benzene-1,2-diamine

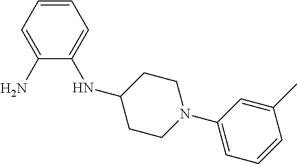 [SMILES: Cc1cccc(N2CCC(Nc3ccccc3N)CC2)c1]

To a suspension of (2-Nitro-phenyl)-(1-m-tolyl-piperidin-4-yl)-amine (step 3, 893 mg, 2.87 mmol) in EtOH (29 mL) was added SnCl₂.2H₂O (3300 mg, 14.3 mmol). The resulting reaction mixture was stirred for 6 hours at 70° C. The reaction mixture was then cooled and concentrated under reduced pressure to a volume of about 5 mL. It was then carefully quenched with saturated sodium bicarbonate solution (50 mL). The resulting suspension was treated with EtOAc (50 mL) and stirred to dissolution of the solids. The mixture was filtered through diatomaceous earth which was subsequently washed with EtOAc (2×20 mL). The organic phase was then separated from the biphasic filtrate and the aqueous portion was further extracted with EtOAc (2×50 mL). The combined organic phases were washed with brine (100 mL), dried over Na₂SO₄, filtered and evaporated to dryness giving 602 mg of the title compound as an off-white solid. HPLC/MS (Method A) t_{R}0.86 minute, M+H 282.1. 1H NMR (DMSO-d₆) Ppm 1.50 (q, 2H) 1.99 (bd, 2H) 2.25 (s, 3H) 2.83 (t, 2H) 3.38 (m, 1H) 3.68 (d, 2H) 4.19 (d, 1H) 4.50 (s, 2H) 6.41 (t, 1H) 6.52 (m, 4H) 6.76 (m, 2H) 7.08 (t, 1H).

### Step 3: (2-Nitro-phenyl)-(1-m-tolyl-piperidin-4-yl)-amine

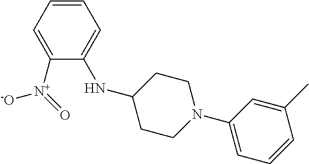

A mixture composed of 2-fluoro-1-nitro-benzene (400 μL, 3.78 mmol), 1-(3-methylphenyl)piperidine-4-amine (876 mg, 3.78 mmol) and NEt₃ (1.06 mL, 7.57 mmol), was heated to 180° C. for 90 minutes under microwave irradiation. While cooling, the reaction mixture became a thick solid. This solid was triturated in a mixture of aqueous HCl 1M (15 mL) and water (15 mL). The resulting suspension was filtered and washed with water affording the title compound as a yellow solid after thorough drying, 893 mg, 68%. HPLC/MS (Method A) t_{R}1.54 minute, M+H 312. 1H NMR (DMSO-d₆) Ppm 1.83 (bs, 2H) 2.15 (m, 2H) 2.30 (s, 3H) 3.19 (bs, 2H) 3.61 (m, 5H) 3.93 (bs, 1H) 6.74 (t, 1H) 6.80-7.2 (m, 2H) 7.21 (d, 1H) 7.58 (t, 1H) 7.96 (bd, 1H) 8.10 (d, 1H).

### Example 55

### 1-(2-Chloro-5-fluoro-benzyl)-3-(1-m-tolyl-piperidin-4-yl)-1,3-dihydro-benzoimidazol-2-ylideneamine

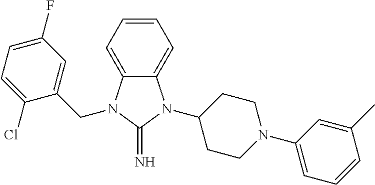 [SMILES: Cc1cccc(N2CCC(n3c(=N)n(Cc4cc(F)ccc4Cl)c4ccccc43)CC2)c1]

The title compound was synthesized in a manner analogous to that used for the synthesis of example 54, using 2-chloro-5-fluoro-benzylbromide instead of 2-chlorobenzylbromide. HPLC/MS (Method A) t_{R}1.35 minute, [(M+2H)/2] 225.0.

### Example 56

### N-(2-{4-[2-Imino-3-(1H-indol-4-ylmethyl)-2,3-dihydro-benzoimidazol-1-yl]-piperidin-1-yl}-4-methyl-phenyl)acetamide

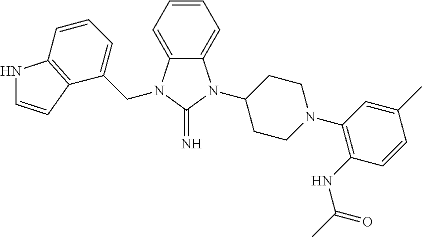 [SMILES: CC(=O)Nc1ccc(C)cc1N1CCC(n2c(=N)n(Cc3cccc4[nH]ccc34)c3ccccc32)CC1]

To a suspension of N-{2-[4-(2-Amino-benzoimidazol-1-yl)-piperidin-1-yl]-4-methyl-phenyl}-acetamide (step 1, 50.0 mg, 0.13 mmol) in MeCN (1.3 mL) were added KI (22.0 mg, 0.13 mmol) and intermediate N (45.0 mg, 0.13 mmol). The reaction vessel was sealed and the reaction mixture was placed on a plate pre heated to 110° C. and stirred at this temperature for 30 minutes. The reaction mixture wasthen cooled to rt and filtered, washing the solid cake with CH₂Cl₂ (2×3 mL). The filtrate was then evaporated to dryness to furnish the product as a crude residue. The residue was purified by reverse-phase preparative HPLC (Method E, using a gradient elution of (MeCN +0.1% TFA) in (Water +0.1% TFA)). Product-containing fractions were pooled and evaporated to dryness. The residue was treated with a CH₂Cl₂/(TFA/Water 98/2): 7/3 solution (2 mL) for 1 hour at rt to remove the Boc protecting group. The medium was evaporated to dryness and partitioned between CH₂Cl₂ (2 mL) and saturated aqueous sodium bicarbonate solution (2 mL). The organic layer was separated, dried and evaporated to dryness to furnish the title compound as an off-white solid, 13.0 mg (20%). HPLC/MS (Method D) t_{R}2.01 minute, M+H 493.2. 1H NMR (DMSO-d₆) Ppm 1.86 (d, 2H) 2.16 (s, 3H) 2.29 (s, 3H) 2.63-2.74 (m, 2H) 2.85 (t, 2H) 3.17 (d, 2H) 4.63 (m, 1H) 5.45 (s, 2H) 6.61 (br. s., 1H) 6.78 (d, 1H) 6.87 (d, 1H) 6.92-6.97 (m, 2H) 6.98 (s, 1H) 7.00-7.07 (m, 2H) 7.35 (d, 1H) 7.38 (t, 1H) 7.71 (d, 1H) 7.74 (d, 1H) 9.04 (s, 1H) 11.26 (s, 1H).

### Step 1: N-{2-[4-(2-Amino-benzoimidazol-1-yl)-piperidin-1-yl]-4-methyl-phenyl}-acetamide

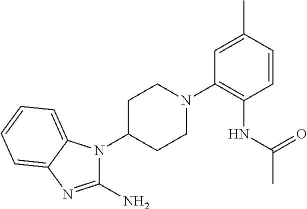 [SMILES: CC(=O)Nc1ccc(C)cc1N1CCC(n2c(N)nc3ccccc32)CC1]

A mixture of N-{2-[4-(2-Amino-phenylamino)-piperidin-1-yl]-4-methyl-phenyl}-acetamide (step 2, 240 mg, 0.71 mmol) and cyanic bromide (5M solution in MeCN, 149 μL, 0.74 mmol) in MeCN (15 mL) and water (1 mL) was stirred for 23 hrs at rt. The reaction mixture was then

diluted with EtOAc (50 mL) and extracted with saturated sodium bicarbonate solution (3×25 mL) and brine (25 mL). The organic phase was then dried over Na₂SO₄, filtered and evaporated to dryness to furnish the title compound as a red-brown resin which was used in the next step without further purification, 268 mg (95% purity based on a quantitative reaction and UV diode array purity). HPLC/MS (Method A) t_{R}0.91 minute, M−H 362.3.

### Step 2: N-{2-[4-(2-Amino-phenylamino)-piperidin-1-yl]-4-methyl-phenyl}-acetamide

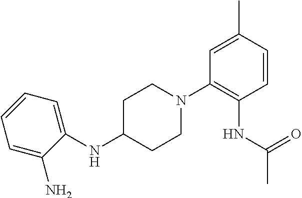 [SMILES: CC(=O)Nc1ccc(C)cc1N1CCC(Nc2ccccc2N)CC1]

To a solution of N-{4-Methyl-2-[4-(2-nitro-phenylamino)-piperidin-1-yl]-phenyl}-acetamide (step 3, 352 mg, 0.955 mmol) in MeOH (9.5 mL) under one atmosphere of hydrogen was added Pd/C (10% w/w, 51 mg, 0.048 mmol). The mixture was stirred at rt for 4.5 hrs. The reaction mixture was filtered through a pad of celite which was subsequently washed with MeOH (2×10 mL). The filtrate was filtered again through a 0.2 micron PTFE membrane to remove residual particles before a final evaporation to furnish the title compound, 240 mg (74%). HPLC/MS (Method A) t_{R}0.96 minute, M+H 339.2.

### Step 3: N-{4-Methyl-2-[4-(2-nitro-phenylamino)-piperidin-1-yl]-phenyl}-acetamide

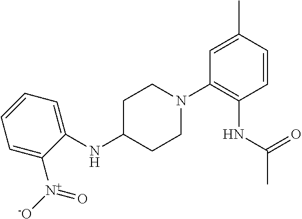

To a solution of N-[2-(4-Amino-piperidin-1-yl)-4-methyl-phenyl]-acetamide (step 4, 290 mg, 1.17 mmol) in MeCN (3.5 mL) was added DIPEA (1380 mg, 10.66 mmol) followed by 2-fluoro-1-nitro-benzene (150 mg, 1.07 mmol). The reaction mixture was then stirred at 150° C. in a sealed vessel for 135 minutes. The reaction mixture was diluted with water (30 mL) and then acidified with concentrated HCl (35% in water, 2 mL). The resulting mixture was then extracted with CH₂Cl₂ (5×15 mL). The combined extracts were evaporated to dryness. The residue was purified by chromatography on silica gel, using a 10% to 100% elution gradient of eluent B (EtOAc) in eluent A (Heptane), yielding the title product as a yellow resin, 352 mg (90%). HPLC/MS (Method A) t_{R}1.62 minute, M+H 369.1.

### Step 4: N-[2-(4-Amino-piperidin-1-yl)-4-methyl-phenyl]acetamide

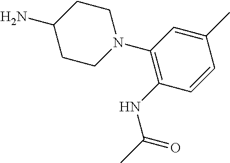 [SMILES: CC(=O)Nc1ccc(C)cc1N1CCC(N)CC1]

To a solution of 1-(2-Acetylamino-5-methyl-phenyl)piperidin-4-yl]-carbamic acid tert-butyl ester (step 5, 902 mg, 2.60 mmol) in CH₂Cl₂ (16 mL) was added a mixture of TFA and water (98/2, 4 mL). The resulting solution was stirred at rt for one hour. The reaction mixture was evaporated to dryness and then co-evaporated several times with toluene to ensure removal of the residual TFA. The resulting title compound was used as is in the next step, 640 mg (quantitative). HPLC/MS (Method A) t_{R}0.67 minute, M+H 248.1.

### Step 5: [1-(2-Acetylamino-5-methyl-phenyl)piperidin-4-yl]-carbamic acid tert-butyl ester

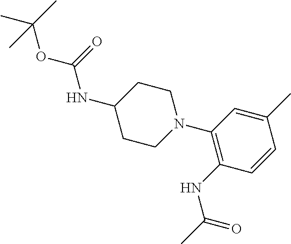 [SMILES: CC(=O)Nc1ccc(C)cc1N1CCC(NC(=O)OC(C)(C)C)CC1]

A solution of [1-(2-Amino-5-methyl-phenyl)-piperidin-4-yl]-carbamic acid tert-butyl ester (step 6, 1.435 g, 85% pure, 3.99 mmol) in EtOAc (8.0 mL) was treated with AcCl (0.69 g, 8.79 mmol) and NEt₃ (0.89 g, 8.79 mmol) and then heated at 80° C. for 1 hr. The reaction was filtered off and the cake was thouroughly washed with EtOAc (2×10 mL). The filtrate was then extracted with 0.1 N aqueous sodium hydroxide solution (2×20 mL) and brine (20 mL). The organic phase was then dried over Na₂SO₄, filtered and evaporated to dryness. The residue was purified by chromatography on silica gel, using a 3/2 mixture of eluent B (EtOAc/NH4OH:99/1) and eluent A (Heptane), yielding the title product as a clear resin, 902 mg (65%). HPLC/MS (Method A) t_{R}1.31 minute, M+H 348.2.

### Step 6: [1-(2-Amino-5-methyl-phenyl)piperidin-4-yl]-carbamic acid tert-butyl ester

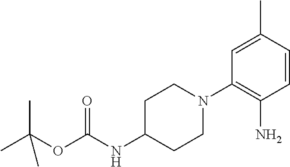 [SMILES: Cc1ccc(N)c(N2CCC(NC(=O)OC(C)(C)C)CC2)c1]

To a solution of [1-(5-Methyl-2-nitro-phenyl)piperidin-4-yl]-carbamic acid tert-butyl ester (step 7, 1612 mg, 4.085 mmol) in MeOH (41 mL) under one atmosphere of hydrogen was added Pd/C (10% w/w, 435 mg, 0.409 mmol). The mixture was stirred at rt for 90 minutes. The reaction mixture was filtered through a pad of celite which was subsequently washed with MeOH (2×10 mL). The filtrate was filtered again through a 0.2 micron PTFE membrane to remove residual particles before a final evaporation to furnish the title compound, 1435 mg (85% purity based on a quantitative reaction), polluted with residual 4-(Boc-amino)piperidine form the last step. HPLC/MS (Method A) t_{R}1.14 minute, M+H 306.1.

### Step 7: [1-(5-Methyl-2-nitro-phenyl)piperidin-4-yl]-carbamic acid tert-butyl ester

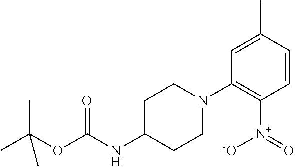

4-(Boc-amino)piperidine (0.949 g, 4.74 mmol), 3-fluoro-4-nitrotoluene (639 mg, 4.121 mmol) and cesium carbonate (1.611 g, 4.95 mmol) were mixed together in MeCN (13.74 ml). The resulting reaction mixture was shaken for 18 hours at rt and for 2 hours at 50° C. The reaction mixture was quenched with water (50 mL) and the resulting suspension was shaken for 10 minutes at rt before the addition of CH₂Cl₂ (30 mL). Shaking was resumed for 10 minutes. The phases were separated and the aqueous portion was further extracted with CH₂Cl₂ (2×30 mL). The combined organic phases were then dried over Na₂SO₄, filtered and evaporated to dryness, leading to the title product, 1612 mg, 100% UV purity, 85% purity as judged by mass balance, the impurity being unreacted 4-(Boc-amino)piperidine. The product was used without further purification. HPLC/MS (Method A) t_{R}1.67 minute, M+H 336.1.

The following examples 56.1 to 56.19 were synthesized in a manner analogous to that used for the synthesis of example 56, using a combination of the following modifications: either 2-fluoro-4-methoxy-1-nitro-benzene or 2-fluoro-1-nitro-benzene or 2-fluoro-1-nitro-4-trifluoromethyl-benzene or 3-fluoro-4-nitro-benzonitrile instead of 3-fluoro-4-nitrotoluene in step 7; 2,4-difluoro-1-nitrobenzene instead of 2-fluoro-1-nitro-benzene in step 3; and either 2-chlorobenzyl bromide (in which case the TFA treatment was omitted since no Boc deprotection was necessary) or intermediate AH instead of intermediate N.

| Ex. | structure | name | HPLC* | MS** | 1H NMR*** |
|---|---|---|---|---|---|
| 56.1 | 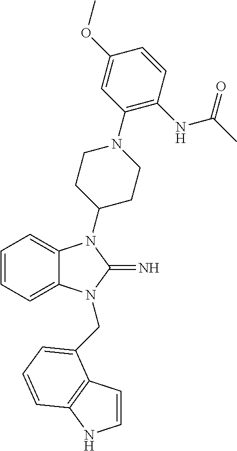 [SMILES: COc1ccc(NC(C)=O)c(N2CCC(n3c(=N)n(Cc4cccc5[nH]ccc45)c4ccccc43)CC2)c1] | N-(2-{4-[2-Imino- 3-(1H-indol-4- ylmethyl)-2,3-dihydro- benzoimidazol-1-yl]- piperidin-1-yl}-4- methoxy-phenyl)- acetamide | 1.87 (D) | 509.2 | 1.76 (d, 2H) 2.11 (s, 3H) 2.64 (q, 2H) 2.83 (t, 2H) 3.18 (d, 2H) 3.75 (s, 3H) 4.57 (m, 1H) 5.30 (s, 2H) 6.61-6.90 (m, 7H) 7.01 (t, 1H) 7.35 (m, 2H) 7.49 (d, 1H) 7.64 (d, 1H) 8.96 (s, 1H) 11.17 (s, 1H) |
| 56.2 | 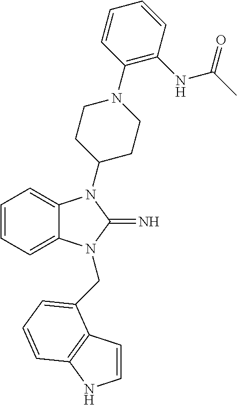 [SMILES: CC(=O)Nc1ccccc1N1CCC(n2c(=N)n(Cc3cccc4[nH]ccc34)c3ccccc32)CC1] | N-(2-{4-[2-Imino- 3-(1H-indol-4- ylmethyl)-2,3-dihydro- benzoimidazol-1-yl]- piperidin-1-yl}- phenyl)-acetamide | 1.89 (D) | 479.2 | 1.80 (d, 2H) 2.17 (s, 3H) 2.58-2.75 (m, 2H) 2.86 (t, 2H) 3.16 (d, 2H) 4.48- 4.69 (m, 1H) 5.34 (s, 2H) 6.61 (br. s., 1H) 6.76-6.87 (m, 3H) 6.87-6.97 (m, 1H) 6.98-7.15 (m, 3H) 7.15-7.23 (m, 1H) 7.28-7.38 (m, 2H) 7.53 (d, 1H) 7.89 (d, 1H) 9.08 (s, 1H) 11.18 (br. s., 1H) |
| 56.3 | 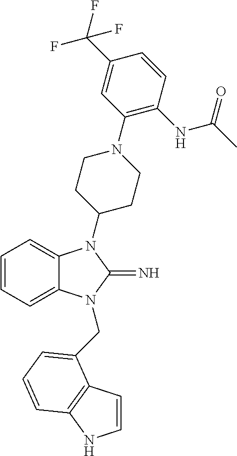 [SMILES: CC(=O)Nc1ccc(C(F)(F)F)cc1N1CCC(n2c(=N)n(Cc3cccc4[nH]ccc34)c3ccccc32)CC1] | N-(2-{4-[2-Imino- 3-(1H-indol-4- ylmethyl)-2,3-dihydro- benzoimidazol-1-yl]- piperidin-1-yl}-4- trifluoromethyl- phenyl)-acetamide | 2.29 (D) | 547.1 | |
| 56.4 | 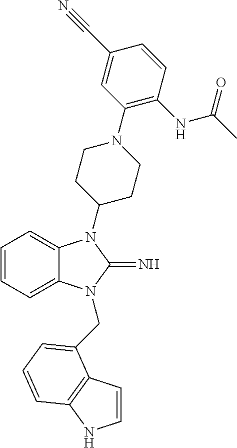 [SMILES: CC(=O)Nc1ccc(C#N)cc1N1CCC(n2c(=N)n(Cc3cccc4[nH]ccc34)c3ccccc32)CC1] | N-(4-Cyano-2-{4- [2-imino-3-(1H- indol-4-ylmethyl)-2,3- dihydro- benzoimidazol-1-yl]- piperidin-1-yl}- phenyl)- acetamide | 1.95 (D) | 504.1 | |
| 56.5 | 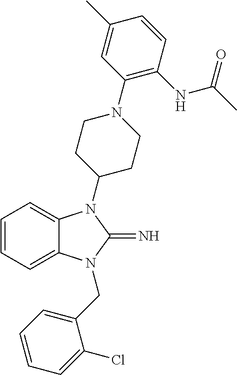 [SMILES: CC(=O)Nc1ccc(C)cc1N1CCC(n2c(=N)n(Cc3ccccc3Cl)c3ccccc32)CC1] | N-(2-{4-[3-(2- Chloro-benzyl)-2- imino-2,3-dihydro- benzoimidazol-1- yl]-piperidin-1-yl}-4- methyl-phenyl)- acetamide | 2.16 (D) | 488.1 | 1.78 (d, 2H) 2.15 (s, 3H) 2.28 (s, 3H) 2.67 (q, 2H) 2.84 (t, 2H) 3.14 (d, 2H) 4.52 (bs, 1H) 5.15 (s, 2H) 6.75-6.98 (m, 6H) 7.28 (m, 2H) 7.52 (d, 2H) 7.73 (d, 1H) 9.00 (s, 1H). |
| 56.6 | 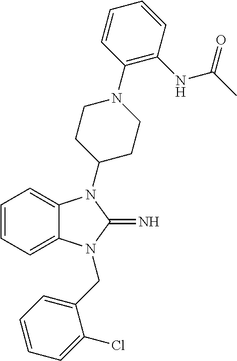 [SMILES: CC(=O)Nc1ccccc1N1CCC(n2c(=N)n(Cc3ccccc3Cl)c3ccccc32)CC1] | N-(2-{4-[3-(2- Chloro-benzyl)-2- imino-2,3-dihydro- benzoimidazol-1- yl]-piperidin-1-yl}- phenyl)-acetamide | 2.02 (D) | 474.1 | 1.76 (dd, 2 H) 2.17 (s, 3 H) 2.60-2.76 (m, 2 H) 2.78-2.97 (m, 2 H) 3.14 (d, 2 H) 4.37-4.76 (m, 1 H) 5.08 (s, 2 H) 6.76-6.97 (m, 3 H) 6.99-7.12 (m, 2 H) 7.18 (d, 1 H) 7.22- 7.38 (m, 5 H) 7.46 (d, 1 H) 7.88 (d, 1 H) 9.07 (s, 1 H) |
| 56.7 | 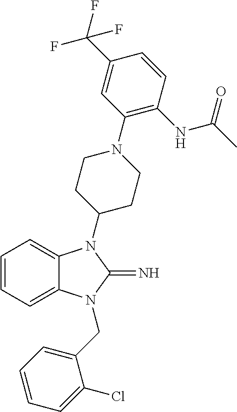 [SMILES: CC(=O)Nc1ccc(C(F)(F)F)cc1N1CCC(n2c(=N)n(Cc3ccccc3Cl)c3ccccc32)CC1] | N-(2-{4-[3-(2- Chloro-benzyl)-2- imino-2,3-dihydro- benzoimidazol-1- yl]-piperidin-1-yl}-4- trifluoromethyl- phenyl)-acetamide | 2.41 (D) | 542.1- 544.1 | 1.81 (d, 2 H) 2.17- 2.30 (m, 3 H) 2.58- 2.80 (m, 2 H) 2.93 (t, 2 H) 3.10-3.27 (m, 2 H) 4.41-4.70 (m, 1 H) 5.15 (s, 2 H) 5.88 (br. s., 1 H) 6.77 (d, 1 H) 6.86 (t, 2 H) 6.95 (t, 1 H) 7.16-7.36 (m, 2 H) 7.38-7.48 (m, 2 H) 7.48-7.61 (m, 2H) 8.16 (d, 1 H) 9.32 (s, 1 H) |
| 56.8 | 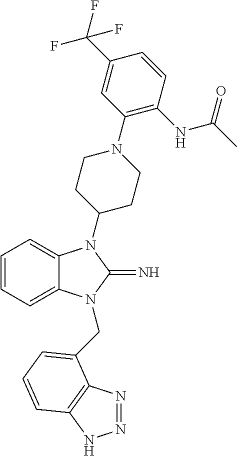 [SMILES: CC(=O)Nc1ccc(C(F)(F)F)cc1N1CCC(n2c(=N)n(Cc3cccc4[nH]nnc34)c3ccccc32)CC1] | N-(2-{4-[3-(1H- Benzotriazol-4- ylmethyl)-2- imino-2,3- dihydro- benzoimidazol- 1-yl]-piperidin- 1-yl}-4- trifluoromethyl- phenyl)- acetamide | 2.16 (D) | 549.2 | |
| 56.9 | 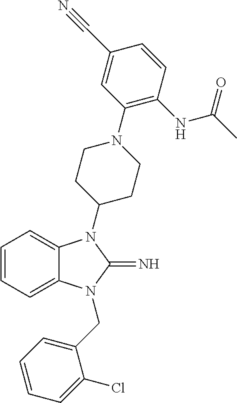 [SMILES: CC(=O)Nc1ccc(C#N)cc1N1CCC(n2c(=N)n(Cc3ccccc3Cl)c3ccccc32)CC1] | N-(2-{4-[3-(2- Chloro-benzyl)-2- imino-2,3-dihydro- benzoimidazol-1- yl]-piperidin-1-yl}-4- cyano-phenyl)- acetamide | 2.11 (D) | 499.1 | |
| 56.10 | 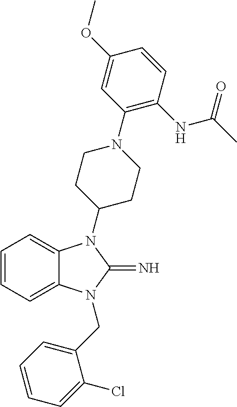 [SMILES: COc1ccc(NC(C)=O)c(N2CCC(n3c(=N)n(Cc4ccccc4Cl)c4ccccc43)CC2)c1] | N-(2-{4-[3-(2- Chloro-benzyl)-2- imino-2,3-dihydro- benzoimidazol-1- yl]-piperidin-1-yl}-4- methoxy-phenyl)- acetamide | 2.05 (D) | 504.1 | (1.78 (d, 2H) 2.12 (s, 3H) 2.67 (q, 2H) 2.83 (t, 2H) 3.18 (d, 2H) 3.75 (s, 3H) 4.53 (m, 1H) 5.14 (s, 2H) 6.61-6.98 (m, 6H) 7.28 (m, 2H) 7.52 (d, 2H) 7.64 (d, 1H) 8.97 (s, 1H). |
| 56.11 | 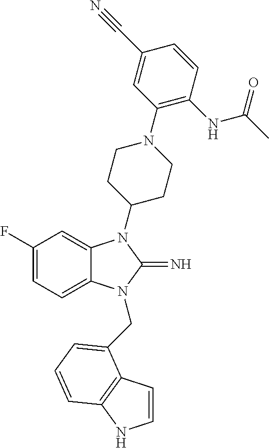 [SMILES: CC(=O)Nc1ccc(C#N)cc1N1CCC(n2c(=N)n(Cc3cccc4[nH]ccc34)c3ccc(F)cc32)CC1] | N-(4-Cyano-2-{4- [6-fluoro-2-imino-3- (1H-indol-4- ylmethyl)-2,3- dihydro- benzoimidazol-1- yl]-piperidin-1-yl}- phenyl)- acetamide | 2.03 (D) | 522.2 | |
| 56.12 | 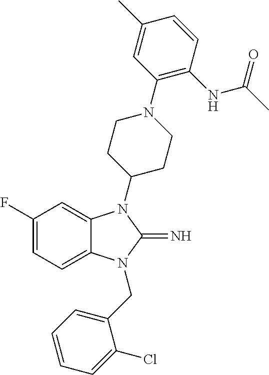 [SMILES: CC(=O)Nc1ccc(C)cc1N1CCC(n2c(=N)n(Cc3ccccc3Cl)c3ccc(F)cc32)CC1] | N-(2-{4-[3-(2- Chloro-benzyl)-6- fluoro-2-imino-2,3- dihydro- benzoimidazol- 1-yl]-piperidin-1- yl}-4-methyl- phenyl)-acetamide | 2.22 (D) | 506.1 | |
| 56.13 | 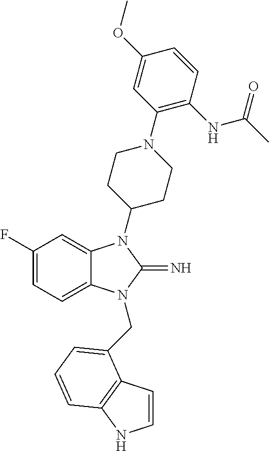 [SMILES: COc1ccc(NC(C)=O)c(N2CCC(n3c(=N)n(Cc4cccc5[nH]ccc45)c4ccc(F)cc43)CC2)c1] | N-(2-{4-[6-Fluoro- 2-imino-3-(1H- indol-4-ylmethyl)-2,3- dihydro- benzoimidazol-1-yl]- piperidin-1-yl}-4- methoxy-phenyl)- acetamide | 1.95 (D) | 527.2 | |
| 56.14 | 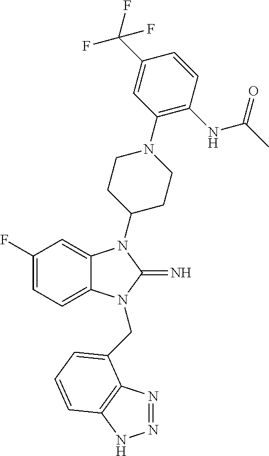 [SMILES: CC(=O)Nc1ccc(C(F)(F)F)cc1N1CCC(n2c(=N)n(Cc3cccc4[nH]nnc34)c3ccc(F)cc32)CC1] | N-(2-{4-[3-(1H- Benzotriazol-4- ylmethyl)-6-fluoro-2- imino-2,3-dihydro- benzoimidazol-1- yl]-piperidin-1-yl}- 4-trifluoromethyl- phenyl)-acetamide | 2.14 (D) | 567.1 | |
| 56.15 | 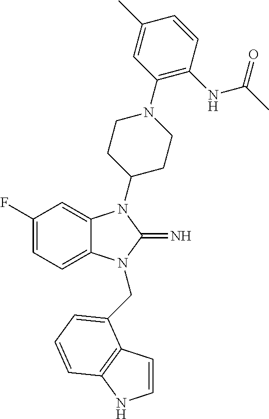 [SMILES: CC(=O)Nc1ccc(C)cc1N1CCC(n2c(=N)n(Cc3cccc4[nH]ccc34)c3ccc(F)cc32)CC1] | N-(2-{4-[6-Fluoro- 2-imino-3-(1H- indol-4-ylmethyl)- 2,3-dihydro- benzoimidazol-1-yl]- piperidin-1-yl}-4- methyl-phenyl)- acetamide | 2.09 (D) | 511.2 | 1.77 (d, 2 H) 2.15 (s, 3 H) 2.28 (s, 3 H) 2.55-2.72 (m, 2 H) 2.83 (t, 2 H) 3.15 (d, 2 H) 4.47- 4.71 (m, 1H) 5.33 (br. s., 2 H) 6.53- 6.77 (m, 3 H) 6.78- 6.90 (m, 2 H) 6.97 (s, 1 H) 7.02 (t, 1 H) 7.26-7.40 (m, 2 H) 7.42-7.58 (m, 1 H) 7.74 (d, 1 H) 9.07 (s, 1H) 11.19 (br. s., 1H) |
| 56.16 | 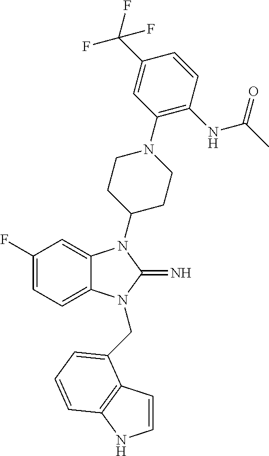 [SMILES: CC(=O)Nc1ccc(C(F)(F)F)cc1N1CCC(n2c(=N)n(Cc3cccc4[nH]ccc34)c3ccc(F)cc32)CC1] | N-(2-{4-[6-Fluoro- 2-imino-3-(1H- indol-4-ylmethyl)- 2,3-dihydro- benzoimidazol-1-yl]- piperidin-1-yl}-4- trifluoromethyl- phenyl)-acetamide | 2.34 (D) | 565.2 | |
| 56.17 | 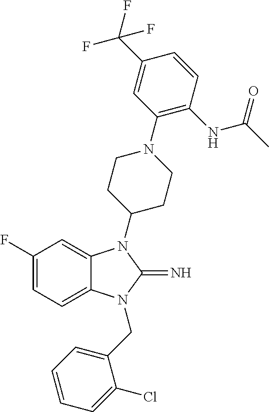 [SMILES: CC(=O)Nc1ccc(C(F)(F)F)cc1N1CCC(n2c(=N)n(Cc3ccccc3Cl)c3ccc(F)cc32)CC1] | N-(2-{4-[3-(2- Chloro-benzyl)-6- fluoro-2-imino-2,3- dihydro- benzoimidazol-1- yl]-piperidin-1- yl}-4- trifluoromethyl- phenyl)-acetamide | 2.46 (D) | 560.2- 562.1 | 1.79 (dd, 2H) 2.23 (s, 3H) 2.58-2.74 (m, 2H) 2.91 (t, 2H) 3.12-3.24 (m, 2 H) 4.60-4.81 (m, 1H) 5.13 (br. s., 2H) 5.82 (br. s., 1H) 6.57-6.75 (m, 2H) 6.77-6.97 (m, 1H) 7.19-7.35 (m, 2H) 7.37-7.47 (m, 2H) 7.47-7.57 (m, 2H) 8.16 (d, 1H) 9.39 (s, 1H) |
| 56.18 | 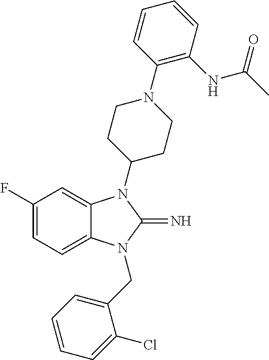 [SMILES: CC(=O)Nc1ccccc1N1CCC(n2c(=N)n(Cc3ccccc3Cl)c3ccc(F)cc32)CC1] | N-(2-{4-[3-(2- Chloro-benzyl)-6- fluoro-2-imino-2,3- dihydro- benzoimidazol- 1-yl]-piperidin-1- yl}-phenyl)- acetamide | 1.95 (D) | 504.1 | |
| 56.19 | 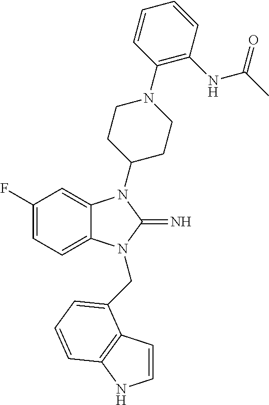 [SMILES: CC(=O)Nc1ccccc1N1CCC(n2c(=N)n(Cc3cccc4[nH]ccc34)c3ccc(F)cc32)CC1] | N-(2-{4-[6-Fluoro- 2-imino-3-(1H- indol-4-ylmethyl)- 2,3-dihydro- benzoimidazol-1-yl]- piperidin-1-yl}- phenyl)-acetamide | 1.96 (D) | 497.2 | |

*t_{R} [min] (method);
**M + H (or specified);
DMSO-d₆ üppm]

### Example 57

### N-(4-Chloro-2-{-4-[2-imino-3-(1H-indol-4-yl)methyl)-2,3-dihydro-benzoimidazol-1-yl]-piperidin-1-yl}-phenyl)-acetamide

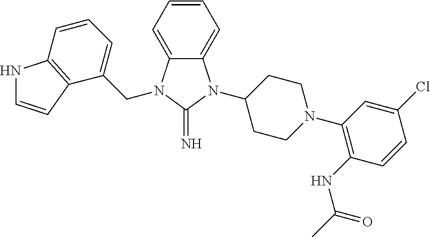 [SMILES: CC(=O)Nc1ccc(Cl)cc1N1CCC(n2c(=N)n(Cc3cccc4[nH]ccc34)c3ccccc32)CC1]

4-{3-[1-(2-Amino-5-chloro-phenyl)-piperidin-4-yl]-2-[tert-butoxycarbonylimino]-2,3-dihydro-benzoimidazol-1-ylmethyl}-indole-1-carboxylic acid tert-butyl ester (step 1, 62 mg, 0.092 mmol) was dissolved in EtOAc (1.84 mL) and acetyl chloride (6.54 μL, 0.092 mmol) was added. The reaction vessel was sealed and placed in a 80° C.-pre-heated oil bath. Stirring was performed for 1 hour before evaporation of the volatiles under reduced pressure. The crude mixture was purified by reverse-phase preparative HPLC (Method E, gradient from 60% (MeOH/MeCN 3/1 +0.1% TFA) in (Water +0.1% TFA) to 90% over 14 minutes). Product-containing fractions were pooled and evaporated to dryness to furnish the boc-protected title compound. Boc deprotection was effected by treatment with TFA (1 mL) for 5 minutes at rt. The mixture was quenched by addition of water (1 mL) and MeOH (1 mL). The volatiles were removed by evaporation, and the residue was partitioned between CH₂Cl₂ (2 mL) and saturated aqueous sodium bicarbonate solution (2 mL). Evaporation of the organic phase after separation and drying afforded the title compound as a white solid, 15 mg (30%). HPLC/MS (Method D) t_{R}2.12 minute, M+H 513.1-515.1. 1H NMR (DMSO-d₆) Ppm 1.75 (br. s., 2H) 2.16 (s, 3H) 2.67 (br. s., 2H) 2.86 (br. s., 2H) 3.17 (br. s., 2H) 4.58 (br.s., 1H) 5.30 (s, 2H) 6.69-7.45 (m., 12H) 7.87 (br. s., 1H) 9.14 (s, 1H) 11.17 (br. s., 1H).

### Step 1: 4-{3-[1-(2-Amino-5-chloro-phenyl)-piperidin-4-yl]-2-[tert-butoxycarbonylimino]-2,3-dihydro-benzoimidazol-1-ylmethyl}-indole-1-carboxylic acid tert-butyl ester

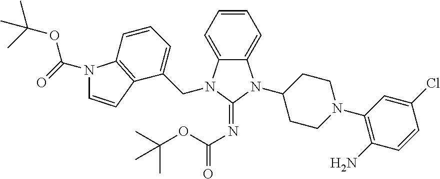 [SMILES: CC(C)(C)OC(=O)/N=c1\n(Cc2cccc3c2ccn3C(=O)OC(C)(C)C)c2ccccc2n1C1CCN(c2cc(Cl)ccc2N)CC1]

Crude 4-{2-[tert-Butoxycarbonylimino]-3-[1-(5-chloro-2-nitro-phenyl)-piperidin-4-yl]-2,3-dihydro-benzoimidazol-1-ylmethyl}-indole-1-carboxylic acid tert-butyl ester (Step 2, assumed mass 320 mg, 0.456 mmol) was dissolved in EtOH (4.56 mL) and tin(II) chloride dihydrate (103 mg, 0.456 mmol) was added. The resulting reaction mixture was then stirred at 50° C. for 2 hours. After 2 hours, tin(II) chloride dihydrate (103 mg, 0.456 mmol) was added and the temperature was raised to 70° C. The reaction was stirred further for 4 hours. The reaction mixture was then carefully poured in saturated aqueous sodium bicarbonate solution (25 mL) and the resulting slurry was vigorously stirred for 15 minutes. EtOAc (10 mL) was then added and the resulting biphasic mixture was stirred for 15 minutes. The resulting paste was filtered through celite and the cake was washed with EtOAc (2×10 mL). The phases were separated and the organic portion was further extracted with saturated aqueous sodium bicarbonate solution (15 mL) and brine (15 mL). The organic phase was then dried over Na₂SO₄, filtered and evaporated to dryness. The residue was purified by chromatography on silica gel, using a 40% to 100% elution gradient of eluent B (EtOAc+1% concentrated NH₄OH) in eluent A (Heptane), yielding two products that could be identified as the title compound (62 mg, 20%, HPLC/MS (Method D) t_{R}3.39 minute, observed ions 230.1, 322.1, 414.1 amu) and a product resulting from the loss of one Boc protecting group (60 mg, 20%, HPLC/MS (Method D) t_{R}2.86 minute, (M+2H)/2 286.1).

### Step 2: 4-{2-[tert-Butoxycarbonylimino]-3-[1-(5-chloro-2-nitro-phenyl)-piperidin-4-yl]-2,3-dihydro-benzoimidazol-1-ylmethyl}-indole-1-carboxylic acid tert-butyl ester

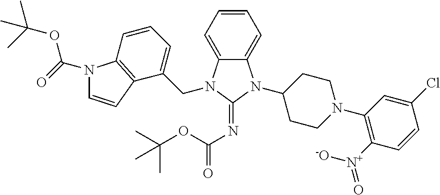

1-[1-(5-Chloro-2-nitro-phenyl)-piperidin-4-yl]-3-(1H-indol-4-ylmethyl)-1,3-dihydro-benzoimidazol-2-ylideneamine (Step 3, 229 mg, 0.457 mmol) was suspended in DCE (1.52 mL) and Boc2O (265 μL, 1.143 mmol) was added followed by DMAP (16.75 mg, 0.137 mmol). The resulting reaction mixture was then stirred at 70° C. for 5 hours. The reaction mixture was diluted with CH₂Cl₂ (15 mL) and extracted with a 10% (wt/wt) aqueous citric acid solution (3×15 mL) and brine (15 mL). The organic phase was dried over Na₂SO₄, filtered and evaporated to dryness to afford the title product as a yellow gum containing excess Boc2O. The gum was used without further purification. HPLC/MS (Method D) t_{R}3.59 minute, M+H 701.3.

### Step 3: 1-[1-(5-Chloro-2-nitro-phenyl)-piperidin-4-yl]-3-(1H-indol-4-ylmethyl)-1,3-dihydro-benzoimidazol-2-ylideneamine

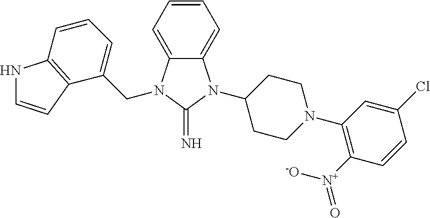

1-(1H-Indol-4-ylmethyl)-3-piperidin-4-yl-1,3-dihydro-benzoimidazol-2-ylideneamine (Step 4, 180 mg, 0.520 mmol) was dissolved in DMF (1.65 mL). Cesium carbonate (194 mg, 0.595 mmol) was added followed by 4-chloro-2-fluoro-1-nitro-benzene (87 mg, 0.496 mmol). The resulting reaction mixture was stirred at rt for 1 hour. The reaction mixture was quenched with water (15 mL) giving a thick colored paste. This paste was filtered and washed with water (2×10 mL). The cake was then dried under high vacuum to afford the title product as a yellow solid, 229 mg (92%). HPLC/MS (Method A) t_{R}1.33 minute, M+H 501.1-503.1.

### Step 4: 1-(1H-Indo)-4-ylmethyl)-3-piperidin-4-yl-1,3-dihydro-benzoimidazol-2-ylideneamine

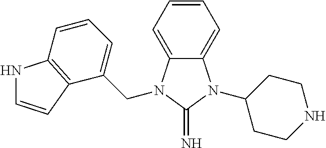 [SMILES: N=c1n(Cc2cccc3[nH]ccc23)c2ccccc2n1C1CCNCC1]

4-[3-(1-tert-Butoxycarbonyl-piperidin-4-yl)-2-imino-2,3-dihydro-benzoimidazol-1-ylmethyl]-indole-1-carboxylic acid tert-butyl ester (Step 5, 341 mg, 0.625 mmol) was dissolved in CH₂Cl₂ (3.36 mL) and a mixture of TFA (1.44 mL, 18.75 mmol) and water (29.5 μL) was added. The resulting reaction mixture was shaken at rt for 30 minutes. The reaction mixture was evaporated to dryness. The residue was diluted with toluene and concentrated three consecutive times to remove residual traces of TFA. The residue was dissolved in CH₂Cl₂/MeOH 1/1 (10 mL) and treated with 2 g of Carbonate resin (Fluka-21850-3.5 mmol/g, pre-washed with CH₂Cl₂ and MeOH) and the resulting suspension was shaken for 20 minutes at rt. The resin was then filtered off and washed with CH₂Cl₂/MeOH 1/1 (2×5 mL). The combined filtrates were evaporated to dryness and then dried under high vacuum to afford the title product, 185 mg (86%). HPLC/MS (Method A) t_{R}0.62 minute, M+H 346.1.

### Step 5: 4-[3-(1-tert-Butoxycarbonyl-piperidin-4-yl)-2-imino-2,3-dihydro-benzoimidazol-1-ylmethyl]-indole-1-carboxylic acid tert-butyl ester

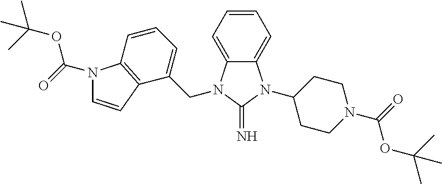 [SMILES: CC(C)(C)OC(=O)N1CCC(n2c(=N)n(Cc3cccc4c3ccn4C(=O)OC(C)(C)C)c3ccccc32)CC1]

Intermediate AI (500 mg, 1.580 mmol) was dissolved in MeCN (15.8 mL) and KI (262 mg, 1.58 mmol) was added followed by intermediate N (545 mg, 1.580 mmol). The reaction vessel was sealed and the mixture was heated to 110° C. for 30 minutes. The reaction mixture was then allowed to cool to rt. The solids were removed by filtration and the filtrate was evaporated to dryness. The residue was purified by chromatography on silica gel, using a 60% to 100% elution gradient of eluent B (EtOAc+1% concentrated NH₄OH) in eluent A (Heptane), yielding the title product as a clear oil, 341 mg (40%). HPLC/MS (Method A) t_{R}1.47 minute, M+H 546.3.

### Example 58

### N-(4-Chloro-2-{4-[3-(2-chloro-benzyl)-2-imino-2,3-dihydro-benzoimidazol-1-yl]-piperidin-1-yl}-phenyl)-acetamide

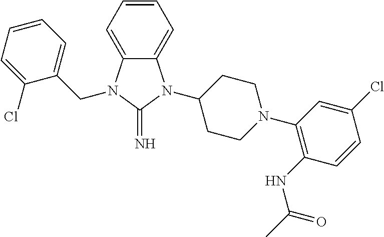 [SMILES: CC(=O)Nc1ccc(Cl)cc1N1CCC(n2c(=N)n(Cc3ccccc3Cl)c3ccccc32)CC1]

4-Chloro-2-{4-[3-(2-chloro-benzyl)-2-imino-2,3-dihydro-benzoimidazol-1-yl]-piperidin-1-yl}-phenylamine (step 1, 292 mg, 0.626 mmol) was dissolved in EtOAc (2.50 mL) and acetyl chloride (44.5 μL, 0.626 mmol) was added. The reaction vessel was sealed and placed in a 80° C.-pre-heated oil bath. Stirring was performed for 1 hour before evaporation of the volatiles under reduced pressure. The crude mixture was purified by reverse-phase preparative HPLC (Method E, gradient from 37% (MeOH/MeCN 3/1 +0.1% TFA) in (Water +0.1% TFA) to 67% over 14 minutes). Product-containing fractions were pooled and evaporated to dryness to furnish the title compound as its TFA salt. The product was desalted by partitioning between CH₂Cl₂ (2 mL) and saturated aqueous sodium bicarbonate solution (2 mL). Evaporation of the organic phase after separation and drying afforded the title compound as a white solid, 34 mg (10%). HPLC/MS (Method D) t_{R}2.24 minute, M+H 508.1-510.1. N-(2-{4-[2-Acetylimino-3-(2-chloro-benzyl)-2,3-dihydro-benzoimidazol-1-yl]-piperidin-1-yl}-4-chloro-phenyl)-acetamide (example 59) was also isolated during the purification by preparative HPLC, 29 mg (8%).

### Step 1: 4-Chloro-2-{4-[3-(2-chloro-benzyl)-2-imino-2,3-dihydro-benzoimidazol-1-yl]-piperidin-1-yl}-phenylamine

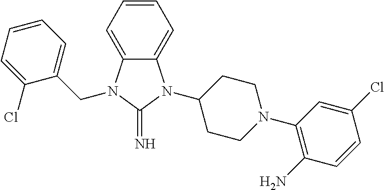 [SMILES: N=c1n(Cc2ccccc2Cl)c2ccccc2n1C1CCN(c2cc(Cl)ccc2N)CC1]

The title compound was synthesized from 1-(2-Chloro-benzyl)-3-[1-(5-chloro-2-nitrophenyl)-piperidin-4-yl]-1,3-dihydro-benzoimidazol-2-ylideneamine (Step 2) in a manner analoguous to that used for the synthesis of 4-{3-[1-(2-Amino-5-chloro-phenyl)-piperidin-4-yl]-2-[tert-butoxycarbonylimino]-2,3-dihydro-benzoimidazol-1-ylmethyl}-indole-1-carboxylic acid tert-butyl ester (example 57, step 1). HPLC/MS (Method A) t_{R}1.36 minute, M+H 496.1-498.1.

### Step 2: 1-(2-Chloro-benzyl)-3-[1-(5-chloro-2-nitro-phenyl)-piperidin-4-yl]-1,3-dihydro-benzoimidazol-2-ylideneamine

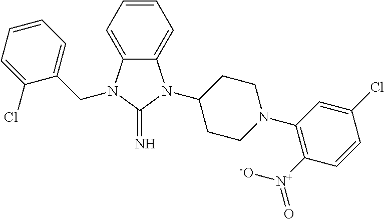

The title compound was synthesized from 1-(2-Chloro-benzyl)-3-piperidin-4-yl-1,3-dihydro-benzoimidazol-2-ylideneamine (Step 3) in a manner analoguous to that used for the synthesis of 1-[1-(5-Chloro-2-nitro-phenyl)-piperidin-4-yl]-3-(1H-indol-4-ylmethyl)-1,3-dihydro-benzoimidazol-2-ylideneamine (example 57, step 3). HPLC/MS (Method A) t_{R}1.36 minute, M+H 496.1-498.1.

### Step 3: 1-(2-Chloro-benzyl)-3-piperidin-4-yl-1,3-dihydro-benzoimidazol-2-ylideneamine

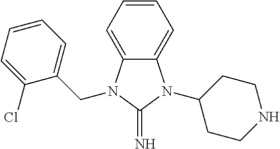 [SMILES: N=c1n(Cc2ccccc2Cl)c2ccccc2n1C1CCNCC1]

The title compound was synthesized from 4-[3-(2-Chloro-benzyl)-2-imino-2,3-dihydro-benzoimidazol-1-yl]-piperidine-1-carboxylic acid tert-butyl ester (Step 4) in a manner analoguous to that used for the synthesis of 1-(1H-Indol-4-ylmethyl)-3-piperidin-4-yl-1,3-dihydro-benzoimidazol-2-ylideneamine (example 57, step 4). HPLC/MS (Method A) t_{R}0.66 minute, M+H 341.2.

### Step 4: 4-[3-(2-Chloro-benzyl)-2-imino-2,3-dihydro-benzoimidazol-1-yl]-piperidine-1-carboxylic acid tert-butyl ester

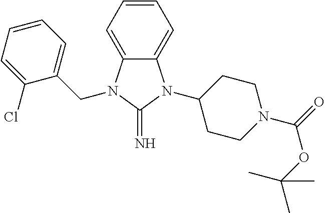 [SMILES: CC(C)(C)OC(=O)N1CCC(n2c(=N)n(Cc3ccccc3Cl)c3ccccc32)CC1]

The title compound was synthesized from intermediate AI in a manner analoguous to that used for the synthesis of 4-[3-(1-tert-Butoxycarbonyl-piperidin-4-yl)-2-imino-2,3-dihydro-benzoimidazol-1-ylmethyl]-indole-1-carboxylic acid tert-butyl ester (example 57, step 5) using 2-chlorobenzylbromide instead of intermediate N. HPLC/MS (Method D) t_{R}2.26 minute, M+H 441.0-443.0.

### Example 59

### N-(2-{4-[2-Acetylimino-3-(2-chloro-benzyl)-2,3-dihydro-benzoimidazol-1-yl]-piperidin-1-yl}-4-chloro-phenyl)-acetamide

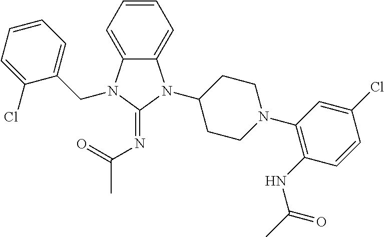 [SMILES: CC(=O)/N=c1\n(Cc2ccccc2Cl)c2ccccc2n1C1CCN(c2cc(Cl)ccc2NC(C)=O)CC1]

The title compound was isolated as a pure product in 8% yield during the synthesis of N-(4-Chloro-2-{4-[3-(2-chloro-benzyl)-2-imino-2,3-dihydro-benzoimidazol-1-yl]-piperidin-1-yl}-phenyl)-acetamide (example 58). HPLC/MS (Method D) t_{R}2.26 minute, M+H 550.2-552.2.

### Example 60

### N-(2-{4-[3-(1H-Benzotriazol-4-ylmethyl)-2-imino-2,3-dihydro-benzoimidazol-1-yl]-piperidin-1-yl}-4-chloro-phenyl)-acetamide

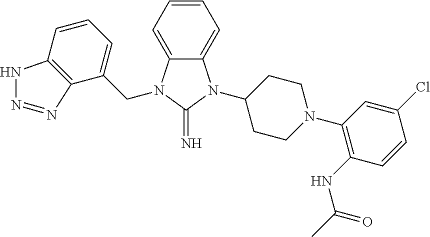 [SMILES: CC(=O)Nc1ccc(Cl)cc1N1CCC(n2c(=N)n(Cc3cccc4[nH]nnc34)c3ccccc32)CC1]

4-{2-tert-Butoxycarbonylimino-3-[1-(5-chloro-2-nitro-phenyl)-piperidin-4-yl]-2,3-dihydro-benzoimidazol-1-ylmethyl}-benzotriazole-1-carboxylic acid tert-butyl ester (step 1, 171 mg, 0.231 mmol) was dissolved in MeOH (2.31 mL) and tin (II) chloride dihydrate (156 mg, 0.693 mmol) was added. The resulting mixture was stirred at rt for 22 hours. More tin (II) chloride dihydrate (156 mg, 0.693 mmol) was added and the reaction was heated up to 50° C. for 8 hours. The mixture was allowed to cool to rt and was carefully quenched with saturated aqueous sodium bicarbonate solution (4 mL). The resulting paste was shaken for 15 minutes at rt. Then EtOAc (3 mL) was added and the biphasic mixture was shaken for a further 15 minutes at rt. The biphasic mixture was then filtered through a pad of celite which was washed with EtOAc (2×4 mL). The phases were separated, the aqueous portion extracted with EtOAc (4 mL), the combined organic layers washed with brine (4 mL), dried over Na2SO4, filtered and evaporated to dryness to give 82 mg of a light yellow solid. The solid was dissolved in EtOAc (1.43 mL). NEt₃ (21.94 μL, 0.157 mmol) was added followed by AcCl (11.19 μL, 0.157 mmol). The resulting mixture was stirred at rt for 30 minutes. AcCl (11.19 μL, 0.157 mmol) and TEA (21.94 μL, 0.157 mmol) were added and the mixture was stirred for 14 hours. AcCl (11.19 μL, 0.157 mmol) and TEA (21.94 μL, 0.157 mmol) were added and the mixture was stirred for 30 minutes. Finally, AcCl (11.19 μL, 0.157 mmol) and TEA (21.94 μL, 0.157 mmol) were added a fourth time and after 30 minutes at rt, the mixture was quenched with MeOH (1 mL) and evaporated to dryness. The crude residue was purified by reverse-phase preparative HPLC (Method E, gradient from 40% (MeOH/MeCN 3/1 +0.1% TFA) in (Water+0.1% TFA) to 70% over 14 minutes). Product-containing fractions were pooled and evaporated to dryness. The residue was then treated with a TFA/DCM 1/3 solution for 1 hour at rt before evaporation to dryness. The resulting crude product was purified a second time by reverse-phase preparative HPLC (Method E, gradient from 40% (MeOH/MeCN 3/1 +0.1% TFA) in (Water +0.1% TFA) to 70% over 14 minutes). Product-containing fractions were pooled, passed through MP carbonate resin cartridges (Stratosphere) to remove the TFA and evaporated to dryness to furnish the title product, 9 mg (12%). HPLC/MS (Method D) t_{R}1.91 minute, M+H 514.8-516.8. 1H NMR (Dimethylsulfoxyde-d6) Ppm 1.90 (d, 2H) 2.17 (s, 3H) 2.57-2.71 (m, 2H) 2.84 (t, 2H) 3.19 (d, 3H) 4.64 (t, 1H) 5.56 (s, 2H) 7.08-7.14 (m, 2H) 7.16 (d, 1H) 7.23 (dd, 2H) 7.32 (d, 1H) 7.66 (br. s., 1H) 7.76 (d, 1H) 7.88 (d, 2H) 9.15 (s, 1H).

### Step 1: 4-{2-tert-Butoxycarbonylimino-3-[1-(5-chloro-2-nitro-phenyl)-piperidin-4-yl]-2,3-dihydro-benzoimidazol-1-ylmethyl}-benzotriazole-1-carboxylic acid tert-butyl ester

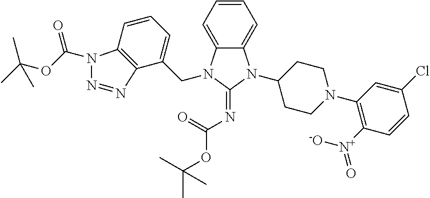

1-(1H-Benzotriazol-4-ylmethyl)-3-[1-(5-chloro-2-nitro-phenyl)-piperidin-4-yl]-1,3-dihydro-benzoimidazol-2-ylideneamine (step 2, 153 mg, 0.304 mmol) was treated with a stock-solution of DMAP (7.43 mg, 0.061 mmol) in DCE (1014 μL). Then Boc2O (353 μL, 1.521 mmol) was added and the resulting mixture was stirred at 70° C. for 3 hours The reaction was cooled down to rt and Boc2O (176 μL, 0.760 mmol) were added. The resulting mixture was stirred for 45 min at rt and diluted up to 5 mL with CH₂Cl₂. This solution was extracted with a 10% aqueous citric acid solution (2×5 mL) and brine (5 mL). The organic phase was then dried over Na2SO4, filtered and evaporated to dryness. The resulting residue was purified by chromatography on silica gel, using a 10% to 55% gradient of eluent B (EtOAc) in eluent A (heptane), yielding the title product as a yellow solid, 171 mg (76%). HPLC/MS (Method D) t_{R}3.33 minute, M+H 702.8.

### Step 2: 1-(1H-Benzotriazol-4-ylmethyl)-3-[1-(5-chloro-2-nitro-phenyl)-piperidin-4-yl]-1,3-dihydro-benzoimidazol-2-ylideneamine

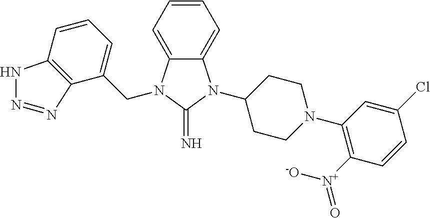

1-[1-(5-Chloro-2-nitro-phenyl)-piperidin-4-yl]-1H-benzoimidazol-2-ylamine (step 3, 300 mg, 0.807 mmol) was dissolved in MeCN (10.80 ml) and treated with intermediate AH (252 mg, 0.807 mmol) and KI (134 mg, 0.807 mmol). The resulting reaction mixture was stirred for 10 minutes at 110° C. under microwave irradiation. The mixture was filtered and the solid materials were washed with CH₂Cl₂ (2×2 mL) and MeOH (2×5 mL). The combined solutions were evaporated to dryness. The resulting residue was purified by chromatography on silica gel, using a 50% to 100% gradient of eluent B (EtOAc/MeOH/conc. NH4OH:90/9/1) in eluent A (heptane/CH₂Cl₂: 1/1), yielding the title product as a solid, 153 mg (34%). HPLC/MS (Method D) t_{R}2.30 minute, M+H 502.8-504.8.

### Step 3: 1-[1-(5-Chloro-2-nitro-phenyl)-piperidin-4-yl]-1H-benzoimidazol-2-ylamine

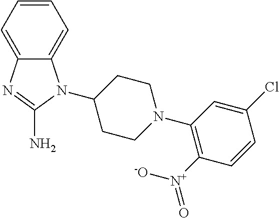

Intermediate AI (1000 mg, 3.16 mmol) was treated with a 3/1 CH₂Cl₂/TFA solution (20 mL) and the resulting reaction mixture was shaken at RT for 75 minutes. The medium was evaporated to dryness The residue was dissolved in MeOH (20 mL) and treated with an excess of MP-carbonate polystyrene resin (10 g, Aldrich 540285-2.5 to 3.5 mmol/g-resin pre-washed with 20 mL MeOH). The suspension was shaken for 15 minutes at rt, after which time the resin was filtered off and washed with MeOH (2×20 mL). The combined filtrates were evaporated to dryness to afford a brown oil (806 mg). A portion of the oil (342 mg) was dissolved in DMF (5.27 mL). Cesium carbonate (772 mg, 2.370 mmol) was added, followed by 4-chloro-2-fluoro-1-nitro-benzene (291 mg, 1.659 mmol) and the resulting reaction mixture was stirred at rt for 5.5 hours. The reaction mixture was poured into water (50 mL). The resulting suspension was vigorously stirred for 15 minutes. It was then filtered and the solid quake further washed with water (2×5 mL) before drying under high vacuum to afford a yellow solid. The solid was purified by chromatography on silica gel, using a 15% to 75% gradient of eluent B (EtOAc/MeOH/conc. NH4OH:90/9/1) in eluent A (heptane/CH₂Cl₂: 1/1), yielding the title product as a yellow-green solid, 563 mg. HPLC/MS (Method D) t_{R}2.08 minute, M+H 372.0-374.0.

### Example 61

### N-(4-Chloro-2-{4-[6-fluoro-2-imino-3-(1H-indol-4-ylmethyl)-2,3-dihydro-benzoimidazol-1-yl]-piperidin-1-yl}-phenyl)-acetamide

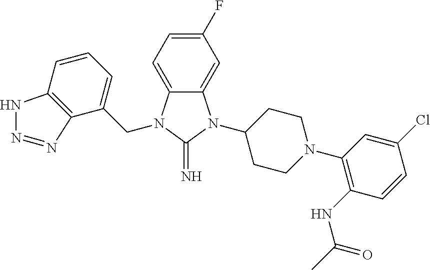 [SMILES: CC(=O)Nc1ccc(Cl)cc1N1CCC(n2c(=N)n(Cc3cccc4[nH]nnc34)c3ccc(F)cc32)CC1]

The title compound was synthesized in a manner analogous to that used for the synthesis of example 57, using intermediate AJ instead of intermediate AI in step 5. HPLC/MS (Method D) t_{R}2.17 minute, M+H 531.2-533.1. 1H NMR (dimethylsulfoxyde-d6) ppm 1.76 (br. s., 2H) 2.17 (s, 3H) 2.67 (br. s., 2H) 2.85 (br. s., 2H) 3.16 (br. s., 2H) 4.60 (br.s., 1H) 5.29 (s, 2H) 6.59-7.45 (m., 13H) 7.88 (br. s., 1H) 9.21 (s, 1H) 11.18 (br. s., 1H)

### Example 62

### N-(4-Chloro-2-{4-[3-(2-chloro-benzyl)-6-fluoro-2-imino-2,3-dihydro-benzoimidazol-1-yl]-piperidin-1-yl}-phenyl)-acetamide

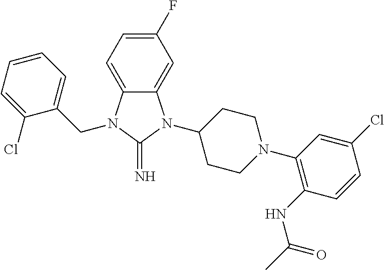 [SMILES: CC(=O)Nc1ccc(Cl)cc1N1CCC(n2c(=N)n(Cc3ccccc3Cl)c3ccc(F)cc32)CC1]

The title compound was synthesized in a manner analogous to that used for the synthesis of example 58, using intermediate AJ instead of intermediate AI in step 4. HPLC/MS (Method D) t_{R}2.31 minute, M+H 526.1-528.1. 1H NMR (dimethylsulfoxyde-d6) ppm 1.78 (m, 2H) 2.18 (s, 3H) 2.64 (m, 2H) 2.85 (m, 2H) 3.17 (d, 2H) 4.56 (m, 1H) 5.13 (s, 2H) 6.62-6.72 (m, 2H) 6.85 (s, 1H) 7.10 (dd, 2H) 7.24-7.34 (m, 2H) 7.52 (dd, 2H) 7.87 (d, 1H) 9.22 (s, 1H).

### Example 63

### N-(2-{4-[3-(1H-Benzotriazol-4-ylmethyl)-6-fluoro-2-imino-2,3-dihydro-benzoimidazol-1-yl]-piperidin-1-yl}-4-chloro-phenyl)-acetamide

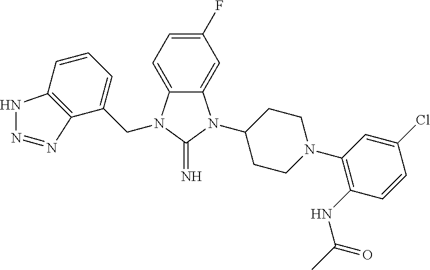 [SMILES: CC(=O)Nc1ccc(Cl)cc1N1CCC(n2c(=N)n(Cc3cccc4[nH]nnc34)c3ccc(F)cc32)CC1]

The title compound was synthesized in a manner analogous to that used for the synthesis of example 60, using intermediate AJ instead of intermediate AI in step 3. HPLC/MS (Method D) t_{R}1.99 minute, M+H 532.8-534.8. 1H NMR (dimethylsulfoxyde-d6) ppm 1.87 (d, 2H) 2.18 (s, 3H) 2.61 (q, 2H) 2.84 (t, 2H) 3.19 (d, 2H) 4.62 (t, 1H) 5.56 (s, 2H) 6.97 (t, 1H) 7.11 (dd, 1H) 7.15 (d, 1H) 7.19-7.30 (m, 2H) 7.44 (br. s., 1H) 7.79 (d, 2H) 7.89 (d, 1H) 9.21 (s, 1H)

### Example 64

### N-(2-{4-[3-(1H-Benzoimidazol-4-ylmethyl)-6-fluoro-2-imino-2,3-dihydro-benzoimidazol-1-yl]-piperidin-1-yl}-4-chloro-phenyl)-acetamide

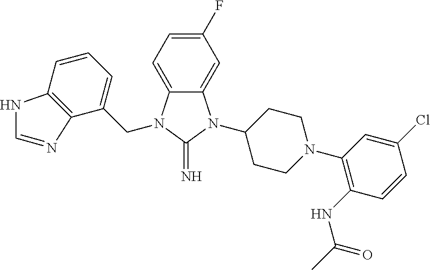 [SMILES: CC(=O)Nc1ccc(Cl)cc1N1CCC(n2c(=N)n(Cc3cccc4[nH]cnc34)c3ccc(F)cc32)CC1]

The title compound was synthesized in a manner analogous to that used for the synthesis of example 60, using intermediate AJ instead of intermediate AI in step 3 and intermediate AG instead of intermediate AH in step 2. HPLC/MS (Method D) t_{R}1.90 minute, M+H 532.0. 1H NMR (dimethylsulfoxyde-d6) ppm 1.78 (d, 2H) 2.17 (s, 3H) 2.54-2.71 (m, 2H) 2.83 (t, 2H) 3.17 (t, 2H) 4.57 (t, 1H) 5.40 (s, 2H) 6.78 (t, 1H) 7.04-7.23 (m, 5H) 7.45-7.64 (m, 2H) 7.87 (d, 1H) 8.32 (s, 1H) 9.21 (s, 1H).

### Example 65

### N-{4-[6-Fluoro-2-imino-3-(1H-indol-4-ylmethyl)-2,3-dihydro-benzoimidazol-1-yl]-6′-methyl-3,4,5,6-tetrahydro-2H-[1,2′]bipyridinyl-3′-yl}-acetamide

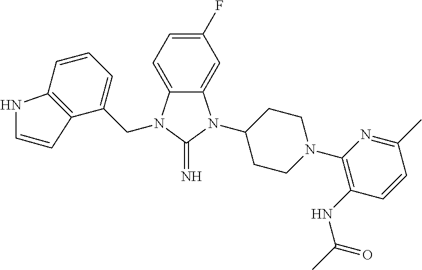 [SMILES: CC(=O)Nc1ccc(C)nc1N1CCC(n2c(=N)n(Cc3cccc4[nH]ccc34)c3ccc(F)cc32)CC1]

The title compound was synthesized in a manner analogous to that used for the synthesis of example 60, combining intermediate AJ and 2-fluoro-6-methyl-3-nitro-pyridine instead of intermediate AI and 4-chloro-2-fluoro-1-nitro-benzene in step 3, and using intermediate N instead of intermediate AH in step 2. HPLC/MS (Method D) t_{R}1.66 minute, [(M+2H)/2] 256.4, M−H 510.0. 1H NMR (dimethylsulfoxyde-d6) ppm 1.71 (d, 2H) 1.75 (s, 1H) 2.12 (s, 3H) 2.37 (s, 3H) 2.53-2.63 (m, 2H) 2.92 (t, 2H) 3.55 (d, 2H) 4.59 (m, 1H) 5.28 (s, 2H) 6.59 (t, 2H) 6.66 (br. s., 1H) 6.83 (br. s., 1H) 6.86 (d, 1H) 7.01 (t, 1H) 7.20-7.37 (m, 3H) 7.89 (d, 1H) 9.20 (s, 1H) 11.18 (br. s., 1H)

### Example 66

### N-(2-{4-[2-Acetylimino-3-(1H-indol-4-ylmethyl)-2,3-dihydro-benzoimidazol-1-yl]-piperidin-1-yl}-4-methyl-phenyl)-acetamide

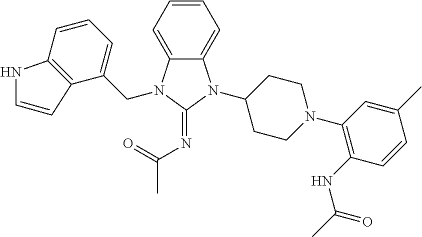 [SMILES: CC(=O)/N=c1\n(Cc2cccc3[nH]ccc23)c2ccccc2n1C1CCN(c2cc(C)ccc2NC(C)=O)CC1]

4-{3-[1-(2-Acetylamino-5-methyl-phenyl)-piperidin-4-yl]-2-imino-2,3-dihydro-benzoimidazol-1-ylmethyl}-indole-1-carboxylic acid tert-butyl ester (step 1, 65 mg, 0.110 mmol) was dissolved in EtOAc (500 μl) and AcCl (11.70 μl, 0.164 mmol) was added. The reaction vessel was sealed, placed on a 80° C. pre-heated plate and stirred at 80° C. for 30 min. Further AcCl (3.9 μl, 0.054 mmol) was added and the reaction mixture was stirred at 80° C. for another 30 minutes. The medium was allowed to cool down, MeOH (2 mL) was added and the resulting solution was evaporated to dryness. The crude residue was purified by reverse-phase preparative HPLC (Method E, gradient from 50% (MeOH/MeCN 3/1 +0.1% TFA) in (Water +0.1% TFA) to 80% over 14 minutes). Product-containing fractions were pooled and evaporated to dryness. The residue was then partitioned between CH₂Cl₂ (5 mL) and saturated aqueous sodium bicarbonate solution (5 mL), the organic phase was evaporated to dryness and treated with TFA (1 mL) for 5 minutes at rt. The medium was quenched by addition of water (1 mL) and MeOH (1 mL) and evaporated to dryness. The residue was partitioned between CH₂Cl₂ (5 mL) and saturated aqueous sodium bicarbonate solution (5 mL), the aqueous portion was extracted with CH₂Cl₂ (2×5 mL), the combined organic phases were dried and evaporated to dryness to furnish the title compound, 10 mg (17%). HPLC/MS (Method A) t_{R}1.16 minute, M+H 535.3. 1H NMR (Dimethylsulfoxyde-d6) Ppm 1.92 (d, 2H) 1.99 (s, 3H) 2.15 (s, 3H) 2.28 (s, 3H) 2.68 (d, 2H) 2.80 (t, 2H) 3.13 (d, 2H) 4.41-4.56 (m, 1H) 5.54 (s, 2H) 6.49 (br. s., 1H) 6.87 (t, 2H) 7.03 (t, 2H) 7.17 (t, 1H) 7.21-7.37 (m, 4H) 7.74 (d, 1H) 8.04 (d, 1H) 9.01 (s, 1H) 11.19 (br. s., 1H)

### Step 1: 4-{3-[1-(2-Acetylamino-5-methyl-phenyl)-piperidin-4-yl]-2-imino-2,3-dihydro-benzoimidazol-1-ylmethyl}-indole-1-carboxylic acid tert-butyl ester

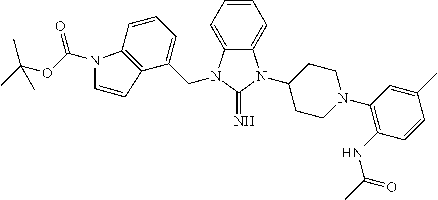 [SMILES: CC(=O)Nc1ccc(C)cc1N1CCC(n2c(=N)n(Cc3cccc4c3ccn4C(=O)OC(C)(C)C)c3ccccc32)CC1]

To a suspension of N-{2-[4-(2-Amino-benzoimidazol-1-yl)-piperidin-1-yl]-4-methyl-phenyl}-acetamide (Example 56 step 1, 132.0 mg, 0.363 mmol) in MeCN (3.6 mL) were added KI (60.3 mg, 0.363 mmol) and intermediate N (125.0 mg, 0.363 mmol). The reaction vessel was sealed and the reaction mixture was placed on a plate pre heated to 110° C. and stirred at this temperature for 30 minutes. The reaction mixture wasthen cooled to rt and filtered, washing the solid cake with EtOAc (2×3 mL). The filtrate was then evaporated to dryness to furnish the product as a crude residue. The residue was purified by chromatography on silica gel, using a 50% to 100% gradient of eluent B (EtOAc+1% NH4OH) in eluent A (heptane), yielding the title product as a solid, 135 mg (60%). HPLC/MS (Method D) t_{R}2.69 minute (95% UV diode array purity), [(M+2H)/2] 297.2.

### Example 67

### Rac-trans-N-(4-Chloro-2-{4-[6-fluoro-2-imino-3-(1H-indol-4-ylmethyl)-2,3-dihydro-benzoimidazol-1-yl]-3-hydroxy-piperidin-1-yl}-phenyl)-acetamide

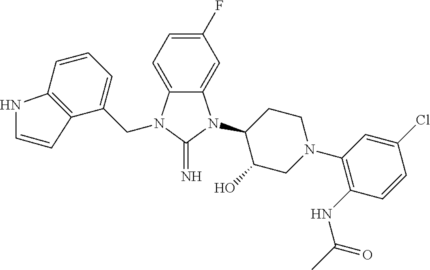 [SMILES: CC(=O)Nc1ccc(Cl)cc1N1CC[C@H](n2c(=N)n(Cc3cccc4[nH]ccc34)c3ccc(F)cc32)[C@@H](O)C1]

Rac-trans-4-{3-[3-Acetoxy-1-(2-amino-5-chloro-phenyl)-piperidin-4-yl]-2-tert-butoxycarbonyl imino-5-fluoro-2,3-dihydro-benzoimidazol-1-ylmethyl}-indole-1-carboxylic acid tert-butyl ester (step 1, 112 mg, 0.222 mmol) was dissolved in ethyl acetate (0.75 mL) and NEt₃ (0.062 mL, 0.444 mmol) was added followed by AcCl (0.032 mL, 0.444 mmol). The vessel was sealed and placed on a 80° C. pre-heated plate and stirred at this temperature for 30 minutes. The reaction was then allowed to cool down to rt. The mixture was diluted with MeOH (1 mL) and the resulting clear solution was stirred for 30 minutes. The volatile materials were removed under reduced pressure and the residue was dried under high vacuum. The residue was then treated with TFA (1000 μL, 12.98 mmol) and the resulting solution was shaken for 5 minutes at rt. The reaction was then quenched with MeOH (1 mL) and water (1 mL). NEt₃ (1 mL) was cautiously added and the resulting solution was treated with saturated aqueous sodium bicarbonate solution (5 mL). The resulting aqueous solution was extracted with EE (3×4 mL). The combined organic layers were then washed with brine (5 mL) and evaporated to dryness. The residue was dissolved in MeOH (1.1 mL) and 1N aqueous sodium hydroxide solution (0.266 mL, 0.266 mmol) was added. The resulting mixture was stirred for 3 hours at rt, after which time the medium was evaporated to dryness. The residue was then dissolved in EtOAc (5 mL) and extracted with water (2×5 mL) and brine (5 mL). The organic layer was then dried over Na₂SO₄, filtered and evaporated to dryness. The crude residue was purified by reverse-phase preparative HPLC (Method E, gradient from 35% (MeOH/MeCN 3/1 +0.1% TFA) in (Water +0.1% TFA) to 65% over 14 minutes). Product-containing fractions were pooled and evaporated to dryness. The still impure product was purified further by chromatography on silica gel, using a 15% to 80% gradient of eluent B (Ethyl acetate/MeOH/NH4OH: 89/10/1) in eluent A (heptane), yielding the title product as a white solid, 17 mg (14%). HPLC/MS (Method D) t_{R}2.22 minute, M+H 547.1-549.1. 1H NMR (dimethylsulfoxyde-d6) Ppm 1.95 (d, 1H) 2.16 (s, 3H) 2.61 (t, 2H) 2.85 (t, 1H) 3.16 (d, 1H) 3.27 (d, 1H) 4.44 (br. s., 1H) 4.52 (br. s., 1H) 5.56 (br. s., 2H) 6.59 (br. s., 1H) 6.72 (d, 1H) 6.88 (t, 1H) 7.01 (t, 2H) 7.11 (dd, 1H) 7.13 (s, 1H) 7.34 (d, 1H) 7.38 (t, 1H) 7.82 (d, 2H) 8.48 (br. s., 1H) 9.30 (br. s., 1H) 11.29 (br. s., 1H).

### Step 1: Rac-trans-4-{3-[3-Acetoxy-1-(2-amino-5-chloro-phenyl)-piperidin-4-yl]-2-tert-butoxycarbonylimino-5-fluoro-2,3-dihydro-benzoimidazol-1-ylmethyl}-indole-1-carboxylic acid tert-butyl ester

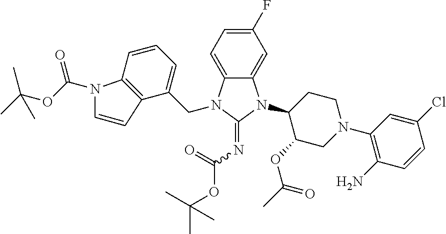 [SMILES: CC(=O)O[C@H]1CN(c2cc(Cl)ccc2N)CC[C@@H]1n1c(=NC(=O)OC(C)(C)C)n(Cc2cccc3c2ccn3C(=O)OC(C)(C)C)c2ccc(F)cc21]

Rac-trans-4-{3-[3-Acetoxy-1-(5-chloro-2-nitro-phenyl)-piperidin-4-yl]-2-tert-butoxycarbonyl imino-5-fluoro-2,3-dihydro-benzoimidazol-1-ylmethyl}-indole-1-carboxylic acid tert-butyl ester (step 2, 116 mg, 0.149 mmol) was dissolved in MeOH (1492 μL) and tin(II) chloride dihydrate (101 mg, 0.448 mmol) was added. The resulting mixture was stirred at rt for 15 hours. The reaction was carefully quenched with saturated acqueous sodium bicarbonate solution (4 mL) and the resulting paste was vigorously shaken for 15 minutes at rt. EtOAc (5 mL) was added and the biphasic mixture was again shaken for 15 minutes. The resulting triphasic mixture was filtered through a pad of celite which was subsequently washed with EtOAc (2×2 mL). The phases were separated and the organic portion was further extracted with saturated acqueous sodium bicarbonate solution (2×5 mL) and brine (5 mL). The organic phase was then dried over Na₂SO₄, filtered and evaporated to dryness to afford the title product, 112 mg (quantitative). HPLC/MS (Method D) t_{R}3.79 minute, (M+2H)/2 374.1.

### Step 2: Rac-trans-4-{3-[3-Acetoxy-1-(5-chloro-2-nitro-phenyl)-piperidin-4-yl]-2-tert-butoxycarbonylimino-5-fluoro-2,3-dihydro-benzoimidazol-1-ylmethyl}-indole-1-carboxylic acid tert-butyl ester

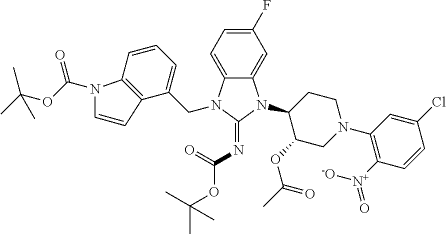

Rac-trans-4-{3-[3-Acetoxy-1-(5-chloro-2-nitro-phenyl)-piperidin-4-yl]-5-fluoro-2-imino-2,3-dihydro-benzoimidazol-1-ylmethyl}-indole-1-carboxylic acid tert-butyl ester (step 3, 100 mg, 0.149 mmol) was suspended in DCE (1492 μL) and DMAP (3.64 mg, 0.030 mmol) was added followed by Boc2O (38.1 μL, 0.164 mmol). The resulting reaction mixture was stirred at 70° C. Further Boc2O (381.0 μL, 1.64 mmol) was added in portion over the course of 26 hours. The reaction was then cooled to rt and diluted with CH₂Cl₂ (5 mL). Imidazole (152 mg, 2.237 mmol) was added (to destroy excess Boc2O) and the reaction mixture was stirred for 30 minutes at rt. The medium was then extracted with 10% aqueous citric acid solution (3×5 mL) and brine (5 mL). The organic layer was dried over Na₂SO₄, filtered and evaporated to dryness yielding the title product, 116 mgs (quantitative). HPLC/MS (Method D) t_{R}3.91 minute, M-Boc+Na 690.8.

### Step 3: Rac-trans-4-{3-[3-Acetoxy-1-(5-chloro-2-nitro-phenyl)-piperidin-4-yl]-5-fluoro-2-imino-2,3-dihydro-benzoimidazol-1-ylmethyl}-indole-1-carboxylic acid tert-butyl ester

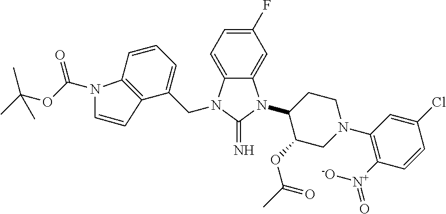

Rac-trans-acetic acid-4-(2-amino-6-fluoro-benzoimidazol-1-yl)-1-(5-chloro-2-nitrophenyl)-piperidin-3-yl ester (step 4, 120 mg, 0.268 mmol) was dissolved in MeCN (2679 μl) at rt. Intermediate N (92 mg, 0.268 mmol) was added followed by KI (44.5 mg, 0.268 mmol) and the resulting reaction mixture was stirred for 10 minutes at 110° C. under microwave irradiation The mixture was then filtered and the solid materials were washed with CH₂Cl₂ (2×2 mL) and MeOH (2×2 mL). The combined filtrates were evaporated to dryness. The residue was purified by chromatography on silica gel, using a 50% isocratic elution of eluent B (EtOAc/MeOH:9/1) in eluent A (heptane), yielding the title product as a solid, 100 mg (56%). HPLC/MS (Method A) t_{R}1.58 minute, M+H 677.1-679.1.

### Step 4: Rac-trans-acetic acid-4-(2-amino-6-fluoro-benzoimidazol-1-yl)-1-(5-chloro-2-nitrophenyl)-piperidin-3-yl ester

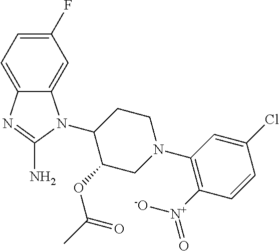

Rac-trans-acetic acid-4-(2-amino-6-fluoro-benzoimidazol-1-yl)-piperidin-3-yl ester (step 5, 284 mg, 0.97 mmol) was dissolved in MeCN (3233 μl) and cesium carbonate (379 mg, 1.164 mmol) was added followed by 2-fluoro-4-chloro-1-nitro-benzene (179 mg, 1.019 mmol). The resulting reaction mixture was stirred at 50° C. for 20 hours. The reaction mixture was poured into water (50 mL) and the resulting suspension was vigorously stirred for 15 minutes. The solids were filtered, washed with water (2×5 mL) and the collected cake was dried overnight under high vacuum to give the title compound as yellow-green solid, 283 mg (65%). HPLC/MS (Method A) t_{R}1.19 minute, M+H 448.0-450.0.

### Step 5: Rac-trans-acetic acid-4-(2-amino-6-fluoro-benzoimidazol-1-yl)-piperidin-3-yl ester

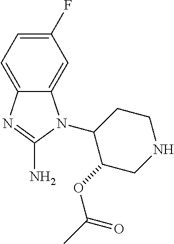 [SMILES: CC(=O)O[C@H]1CNCCC1n1c(N)nc2ccc(F)cc21]

Rac-trans-3-acetoxy-4-(2-amino-6-fluoro-benzoimidazol-1-yl)-piperidine-1-carboxylic acid tert-butyl ester (step 6, 1137 mg, 2.90 mmol) was treated with a CH₂Cl₂ (14 mL)/TFA (6 mL)/water (0.2 mL) solution and the resulting mixture was stirred at rt for 30 minutes. The reaction mixture was evaporated to dryness and the residue was taken up in toluene and concentrated again to remove residual TFA. The residue was then taken up in a CH₂Cl₂/MeOH 1/1 mixture and treated with 5.0 g of tetraalkylammonium carbonate resin (Aldrich 540285, 2.5-3.5 mmol/g nitrogen content). After 15 minutes of gentle stirring, the resin was then filtered off and washed thoroughly with CH₂Cl₂/MeOH 1/1. The combined organic solutions were then evaporated to dryness to yield the title product as a crude oil, 840 mg (quantitative), which was used iwhtout further purification. HPLC/MS (Method A) t_{R}0.41 minute, M+H 293.1.

### Step 6: Rac-trans-3-acetoxy-4-(2-amino-6-fluoro-benzoimidazol-1-yl)-piperidine-1-carboxylic acid tert-butyl ester

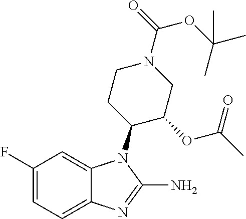 [SMILES: CC(=O)O[C@H]1CN(C(=O)OC(C)(C)C)CC[C@@H]1n1c(N)nc2ccc(F)cc21]

To a solution of rac-trans-3-acetoxy-4-(2-amino-5-fluoro-phenylamino)-piperidine-1-carboxylic acid tert-butyl ester (step 7, 1212 mg, assumed 100% pure for the purpose of calculations, 3.30 mmol) in MeCN (10.3 mL) and water (688 μL) was added cyanic bromide 5 M in MeCN (660 μL, 3.30 mmol) dropwise. The resulting solution was stirred at rt for 16 hours. The medium was then evaporated to dryness. The crude was taken up in EtOAc (25 mL) and washed successively with saturated aqueous sodium bicarbonate solution (2×20 mL) and brine (1×20 mL). The combined organics were dried over Na2SO4 and concentrated to a crude, black resin. The residue was purified by chromatography on silica gel, using a 5% to 100% gradient of eluent B (EtOAc/MeOH/Et3N:89/10/1) in eluent A (heptane/CH₂Cl₂: 1/1), yielding the title product as a dark grey solid, 1.0 g (78%). HPLC/MS (Method D) t_{R}1.70 minute, M+H 393.2. 1H NMR (dimethylsulfoxyde-d6) Ppm 1.46 (s, 9H) 1.74 (br. s., 3H) 1.85 (d, 1H) 2.26-2.43 (m, 1H) 2.66-3.06 (m, 2H) 4.09 (d, 1H) 4.29 (br. s., 1H) 4.45-4.60 (m, 1H) 5.21-5.39 (m, 1H) 6.38 (br. s., 2H) 6.74 (td, 1H) 7.05 (dd, 1H) 7.15 (br. s., 1H).

### Step 7: Rac-trans-3-Acetoxy-4-(2-amino-5-fluoro-phenylamino)-piperidine-1-carboxylic acid tert-butyl ester

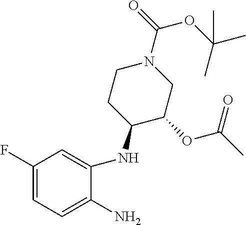 [SMILES: CC(=O)O[C@H]1CN(C(=O)OC(C)(C)C)CC[C@@H]1Nc1cc(F)ccc1N]

To a solution of rac-trans-3-acetoxy-4-(5-fluoro-2-nitro-phenylamino)-piperidine-1-carboxylic acid tert-butyl ester (step 8, 1.55 g, 3.32 mmol) in EtOH (35 mL) was added Pd/C (10% w/w, 0.353 g, 0.332 mmol). The medium was then placed under an atmosphere of hydrogen. The resulting clear solution was stirred at rt for 160 minutes. The medium was then filtered and evaporated to dryness to afford the crude product as a clear oil, 1.30 g. HPLC/MS (Method D) t_{R}2.04 minute (77% UV diode array purity), M+Na 368.1.

### Step 8: Rac-trans-3-acetoxy-4-(5-fluoro-2-nitro-phenylamino)-piperidine-1-carboxylic acid tert-butyl ester

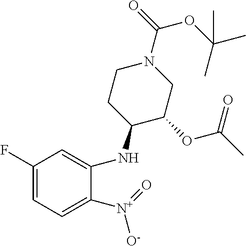 [SMILES: CC(=O)O[C@H]1CN(C(=O)OC(C)(C)C)CC[C@@H]1Nc1cc(F)ccc1[N+](=O)[O-]]

To a solution of Rac-trans-4-(5-fluoro-2-nitro-phenylamino)-3-hydroxy-piperidine-1-carboxylic acid tert-butyl ester (step 9, 1190 mg, 3.35 mmol) in CH₂Cl₂ (11.2 mL) at 0° C. was added DIPEA (877 μL, 5.02 mmol) followed by acetyl chloride (262 μL, 3.68 mmol). The resulting clear solution was allowed to warm up and was stirred at rt for 3 hours. Acetyl chloride (143 μL, 2.009 mmol) and DIPEA (292 μL, 1.674 mmol) were added and stirring was resumed at rt for 210 minutes. Further acetyl chloride (143 μL, 2.009 mmol) and DIPEA (292 μL, 1.674 mmol) were added and the medium was stirred for an additional 45 minutes at rt. The medium was then concentrated to a thick oil. The crude was taken up in diethyl ether (50 mL) and washed successively with 10% aqueous citric acid solution (3×25 mL) and brine (1×25 mL). The combined organics were dried over Na2SO4 and concentrated to a crude oil, 1550 mg, which was used without further purification (93% UV diode array purity). HPLC/MS (Method D) t_{R}3.05 minute, M+Na 420.1. 1H NMR (dimethylsulfoxyde-d6) Ppm 1.42 (s, 9H) 1.69 (d, 1H) 1.89 (s, 3H) 1.93-2.01 (m, 1H) 2.82-3.16 (m, 2H) 3.77 (dt, 1H) 3.93-4.11 (m, 2H) 4.80 (td, 1H) 6.58 (ddd, 1H) 7.14 (dd, 1H) 8.09-8.19 (m, 2H).

### Step 9: Rac-trans-4-(5-fluoro-2-nitro-phenylamino)-3-hydroxy-piperidine-1-carboxylic acid tert-butyl ester

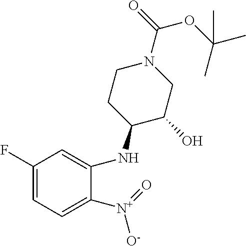 [SMILES: CC(C)(C)OC(=O)N1CC[C@H](Nc2cc(F)ccc2[N+](=O)[O-])[C@@H](O)C1]

To a solution of crude, impure rac-trans-4-amino-3-hydroxy-piperidine-1-carboxylic acid tert-butyl ester (step 10, 2750 mg, assumed molar amount 12.72 mmol based on 100% purity) in iPrOH (20 mL) was added 2,4-difluoro-1-nitro-benzene (1395 μl, 12.72 mmol) followed by sodium carbonate (1617 mg, 15.26 mmol). The resulting mixture was stirred at 150° C. under microwave irradiation for 90 minutes. The medium was filtered and the solid cake was washed with MeOH. The filtrate was evaporated to dryness. The resulting residue was purified by chromatography on silica gel, using a 5% to 40% gradient of eluent B (EtOAc) in eluent A (heptane/CH₂Cl₂: 1/1), yielding the title product as a yellow solid, 1697 mg (38%). HPLC/MS (Method D) t_{R}2.63 minute (100% UV diode array purity), M−H 354.2. 1H NMR (dmso-d6) Ppm 1.39-1.50 (m, 10H) 1.95 (dd, 1H) 2.66 (br. s., 1H) 2.91 (br. s., 1H) 3.39-3.50 (m, 1H) 3.56-3.67 (m, 1H) 3.87 (d, 1H) 3.94-4.10 (m, 1H) 5.38 (d, 1H) 6.53 (ddd, 1H) 7.08 (dd, 1H) 8.10-8.22 (m, 2H).

### Step 10: Rac-trans-4-Amino-3-hydroxy-piperidine-1-carboxylic acid tert-butyl ester

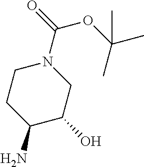

To a solution of 3,6-Dihydro-2H-pyridine-1-carboxylic acid tert-butyl ester (step 11, 7.1 g, 38.7 mmol) in CH₂Cl₂ (77 mL) at 0° C. was added mCPBA (8.68 g, 38.7 mmol) in portions. The resulting clear solution was stirred at 0° C. for 20 min. The medium was then allowed to reach rt. After 3 hours of stirring ar rt, further mCPBA (4.34 g, 19.37 mmol) was added. The medium was stirred at rt for an additional 16 hours. The medium was then diluted with Et2O (500 mL) and washed with saturated aqueous sodium bicarbonate solution (2×200 mL) and brine (250 mL). The organic phase was dried over Na₂SO₄ and concentrated to give a crude brown oil, 9.7 g. The crude oil was dissolved in EtOH (183 ml) and sodium azide (11.87 g, 183 mmol), magnesium sulfate (10.99 g, 91 mmol) and water (3.29 ml, 183 mmol) were successively added. The resulting suspension was refluxed for 16 hr. The medium was then cooled in an ice bath and filtered. The filtrate was evaporated to a residue which was taken up in CH2Cl2 (300 mL), filtered again and concentrated under vacuum to give a brown oil (6.75 g). The oil was dissolved in EtOH (111 mL). Pd/C (10% w/w, 4.74 g, 4.46 mmol) was added and the mixture was stirred under an atmosphere of hydrogen for 16 hours. The medium was finally filtered and evaporated to dryness, yielding the impure title compound as a crude brown oil (5.7 g) containing majorly the desired title compound and a regiosiomer. The crude product was used in the next step without purification.

### Step 11: 3,6-Dihydro-2H-pyridine-1-carboxylic acid tert-butyl ester

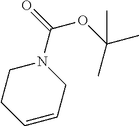

To a solution of 1,2,3,6-tetrahydropyridine (5.52 mL, 60.5 mmol) in MeOH (18.95 mL) at 0° C. was carefully added Boc2O (11.70 mL, 50.4 mmol) in MeOH (7.58 mL). The resulting clear solution was stirred at rt for 30 min. The medium was then concentrated under vacuum to remove the solvent and unreacted starting material, to afford the title product as a brown oil, 7.1 g (77%). No purification was necessary. HPLC/MS (Method A) t_{R}1.44 minute, M+H 184.0. 1H NMR (Chloroform-d) Ppm 1.47 (s, 9H) 2.04-2.20 (m, 2H) 3.48 (t, 2H) 3.82-3.92 (m, 2H) 5.65 (d, 1H) 5.76-5.88 (m, 1H)

The examples 67.1 to 67.7 were synthesized in a manner analogous to that used for the synthesis of example 67, using a combination of the following modifications: 2-fluoro-6-methyl-3-nitropyridine instead of 2-fluoro-4-chloro-1-nitro-benzene in step 4; 2-fluoro-1-nitro-benzene instead of 2,4-difluoro-1-nitrobenzene in step 9; and either 2-chlorobenzyl bromide or intermediate AH or intermediate AG instead of intermediate N in step 3.

| Ex. | structure | name | HPLC* | MS** | 1H NMR*** |
|---|---|---|---|---|---|
| 67.1 | 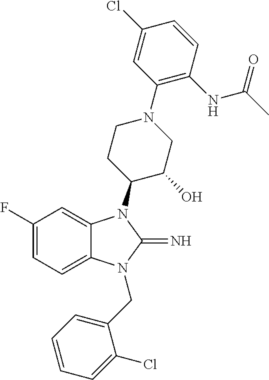 [SMILES: CC(=O)Nc1ccc(Cl)cc1N1CC[C@H](n2c(=N)n(Cc3ccccc3Cl)c3ccc(F)cc32)[C@@H](O)C1] | Rac-trans-N-(4- Chloro-2-{4-[3-(2- chloro-benzyl)-6- fluoro-2-imino-2,3- dihydro-benzo- imidazol-1-yl]-3- hydroxy-piperidin- 1-yl}-phenyl)- acetamide | 2.35 (D) | 542.1- 544.0 | |
| 67.2^{a} | 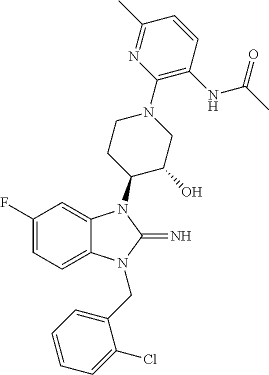 [SMILES: CC(=O)Nc1ccc(C)nc1N1CC[C@H](n2c(=N)n(Cc3ccccc3Cl)c3ccc(F)cc32)[C@@H](O)C1] | Rac-trans-N-{4-[3- (2-Chloro-benzyl)- 6-fluoro-2-imino- 2,3-dihydro- benzoimidazol-1- yl]-3-hydroxy-6′- methyl-3,4,5,6- tetrahydro-2H- [1,2′]bipyridinyl-3′- yl}-acetamide | 2.05 (D) | (M + 2 H)/2 262.1; M − H 521.2 | 1.99 (bd, 1H) 2.15 (s, 3H) 2.4 (s, 3H) 2.71 (t, 1H) 2.91 (t, 1H) 3.57 (d, 1H) 3.70 (d, 1H) 4.51 (m, 2H) 5.46 (s, 2H) 5.6 (bs, 1H) 6.86 (d, 1H) 6.92 (d, 1H) 7.11 (m, 1H) 7.26-7.41 (m, 3H) 7.59 (d, 1H) 7.83 (bs, 1H) 7.94 (d, 1H) 9.24 (bs, 1H) |
| 67.3^{a} | 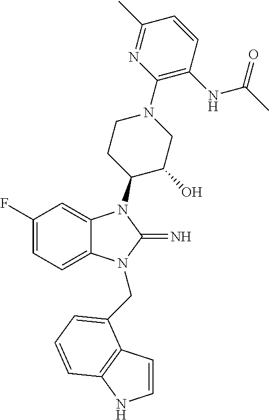 [SMILES: CC(=O)Nc1ccc(C)nc1N1CC[C@H](n2c(=N)n(Cc3cccc4[nH]ccc34)c3ccc(F)cc32)[C@@H](O)C1] | Rac-trans-N-{4-[6- Fluoro-2-imino-3- (1H-indol-4-yl- methyl)-2,3- dihydro-benzo- imidazol-1-yl]-3- hydroxy-6′-methyl- 3,4,5,6- tetrahydro-2H- [1,2′]bipyridinyl- 3′-yl}-acetamide | 1.86 (D) | (M + 2 H)/2 264.5; M − H 526.2 | |
| 67.4^{b} | 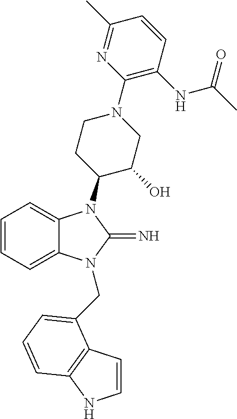 [SMILES: CC(=O)Nc1ccc(C)nc1N1CC[C@H](n2c(=N)n(Cc3cccc4[nH]ccc34)c3ccccc32)[C@@H](O)C1] | Rac-trans-N-{3- Hydroxy-4-[2- imino-3-(1H- indol-4- ylmethyl)-2,3- dihydro- benzoimidazol- 1-yl]-6′-methyl- 3,4,5,6- tetrahydro-2H- [1,2′]bipyridinyl- 3′-yl}-acetamide | 1.30 (D) | 510.1 | |
| 67.5^{b} | 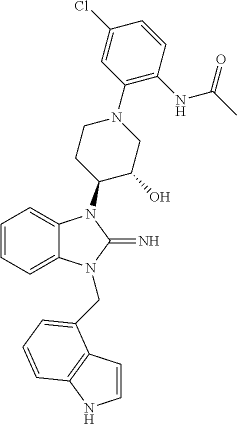 [SMILES: CC(=O)Nc1ccc(Cl)cc1N1CC[C@H](n2c(=N)n(Cc3cccc4[nH]ccc34)c3ccccc32)[C@@H](O)C1] | Rac-trans-N-(4- Chloro-2-{3- hydroxy-4-[2- imino-3-(1H-indol- 4-ylmethyl)-2,3- dihydro- benzoimidazol-1- yl]-piperidin-1-yl}- phenyl)- acetamide | 1.91 (D) | 529.1- 531.1 | |
| 67.6 | 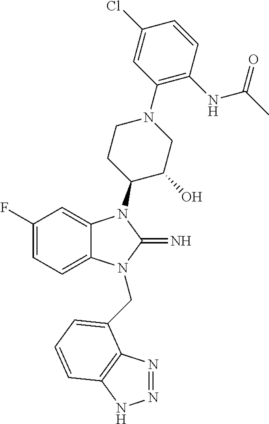 [SMILES: CC(=O)Nc1ccc(Cl)cc1N1CC[C@H](n2c(=N)n(Cc3cccc4[nH]nnc34)c3ccc(F)cc32)[C@@H](O)C1] | Rac-trans-N-(2-{4- [3-(1H-Benzo- triazol-4- ylmethyl)-6-fluoro- 2-imino-2,3- dihydro- benzoimidazol-1- yl]-3-hydroxy- piperidin-1-yl}-4- chloro-phenyl)- acetamide | 2.39 (D) | 548.8- 550.8 | |
| 67.7^{a} | 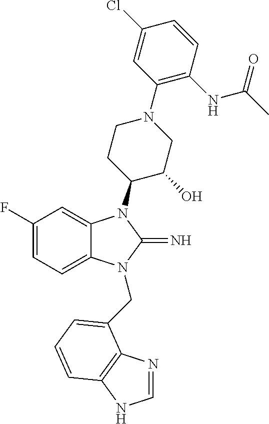 [SMILES: CC(=O)Nc1ccc(Cl)cc1N1CC[C@H](n2c(=N)n(Cc3cccc4[nH]cnc34)c3ccc(F)cc32)[C@@H](O)C1] | Rac-trans-N-(2-{4- [3-(1H-Benzo- imidazol-4- ylmethyl)-6-fluoro- 2-imino-2,3- dihydro-benzo- imidazol-1-yl]-3- hydroxy-piperidin- 1-yl}-4-chloro- phenyl)- acetamide | 1.56 (D) | 548.1- 550.1 | 1.76 (s, 1 H) 1.79 (br. s., 1H) 2.16 (s, 4 H) 2.58 (t, 2H) 2.85 (t, 1H) 3.10 (d, 1H) 3.23 (d, 1H) 4.26 (br. s., 1H) 4.49 (br. s., 1H) 5.16 (br. s., 1H) 5.33 (br. s., 2H) 6.70 (br. s., 1H) 6.98-7.18 (m, 5 H) 7.36- 7.58 (m, 2 H) 7.82 (d, 1H) 8.29 (s, 1H) 9.23 (s, 1H) |

*t_{R} [min] (method);
**M + H (or specified);
***DMSO-d₆ Ppm

### Example 77

### N-{6′-Chloro-4-[3-(2-chloro-benzyl)-6-fluoro-2-imino-2,3-dihydro-benzoimidazol-1-yl]-3,4,5,6-tetrahydro-2H-[1,2′]bipyridinyl-3′-yl}-acetamide

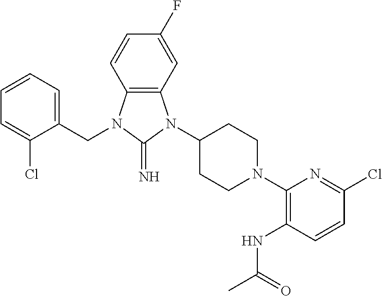 [SMILES: CC(=O)Nc1ccc(Cl)nc1N1CCC(n2c(=N)n(Cc3ccccc3Cl)c3ccc(F)cc32)CC1]

1-(2-Chloro-benzyl)-5-fluoro-3-piperidin-4-yl-1,3-dihydro-benzoimidazol-2-ylideneamine (Intermediate AN, 50 mg, 0.139 mmol) was reacted with N-(6-Chloro-2-fluoro-pyridin-3-yl)-acetamide (step 1, 80 mg, 0.320 mmol) and cesium carbonate (55 mg, 0.167 mmol) in DMF (2 mL) at 80° C. for 24 h. The reaction mixture was filtered and purified by reverse-phase preparative HPLC (Method E, gradient from 20% (MeCN +0.1% TFA) in (Water +0.1% TFA) to 80% over 14 minutes) to give the title compound as a pale purple TFA salt, 22 mg (30%). HPLC/MS (Method B) t_{R}1.00 minute, M+H 527.7-529.7. 1H NMR (Chloroform-d) Ppm 1.93 (d, 2H) 2.28 (s, 3H) 2.83-2.93 (m, 2H) 3.30 (t, 3H) 3.56 (d, 2H) 5.52 (s, 2H) 6.98 (d, 1H) 7.00-7.03 (m, 1H) 7.05-7.08 (m, 1H) 7.11 (d, 1H) 7.24 (t, 1H) 7.32 (t, 1H) 7.43-7.47 (m, 2H) 8.1 (s, br, 1H) 8.51 (d, 1H) 9.2 (s, br, 2H)

### Step 1: N-(6-Chloro-2-fluoro-pyridin-3-yl)-acetamide

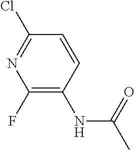

A solution of 6-chloro-2-fluoro-pyridin-3-ylamine (100 mg, 0.482 mmol) in glacial acetic acid (2 mL) was treated with acetyl chloride (69 μL, 0.964 mmol) at rt for 4 h, then at 50° C. for 90 min. The reaction mixture was slowly poured into 100 mL saturated aqueous sodium bicarbonate solution and the product extracted with ethyl acetate (3×70 mL). The combined organic extracts were washed with brine, dried over MgSO4, filtered, and evaporated to give a light purple solid, 95 mg (78%). 1H NMR (Methanol-d4) Ppm 2.22 (s, 3H) 7.46 (d, 1H) 8.16 (d, 1H)

### Example 78

### N-{4-[3-(2-Chloro-benzyl)-6-fluoro-2-imino-2,3-dihydro-benzoimidazol-1-yl]-6′-methyl-3,4,5,6-tetrahydro-2H-[1,2′]bipyridinyl-3′-yl}-acetamide

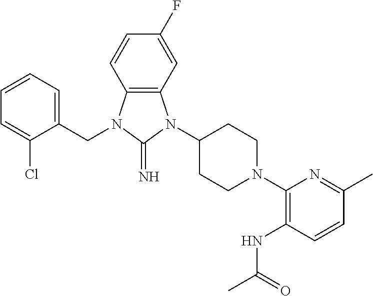 [SMILES: CC(=O)Nc1ccc(C)nc1N1CCC(n2c(=N)n(Cc3ccccc3Cl)c3ccc(F)cc32)CC1]

A suspension of 4-[3-(2-Chloro-benzyl)-6-fluoro-2-imino-2,3-dihydro-benzoimidazol-1-yl]-6′-methyl-3,4,5,6-tetrahydro-2H-[1,2′]bipyridinyl-3′-ylamine (Step 1, 25 mg, 0.054 mmol) in THF (2 mL), ethyl acetate (0.5 mL), and acetic acid (0.5 mL) was treated with acetyl chloride (4 μL, 0.056 mmol) and stirred at 80° C. for 30 min. The solvents were evaporated and the residue was purified by reverse-phase preparative HPLC (Method E, gradient from 20% (MeCN +0.1% TFA) in (Water +0.1% TFA) to 80% over 14 minutes) to give the title compound as a pale purple TFA salt, 2.8 mg (10%). HPLC/MS (Method B) t_{R}1.02 minute, M+H 507.6-509.7. 1H NMR (Methanol-d4) Ppm 2.11 (d, 2H) 2.24 (s, 3H) 2.52 (s, 3H) 2.68-2.84 (m, 2H) 3.20 (t, 2H) 3.80 (d, 2H) 4.65-4.78 (m, 1H) 5.54 (s, 2H) 6.97-7.04 (m, 2H) 7.06-7.13 (m, 1H) 7.24-7.28 (m, 1H) 7.28-7.33 (m, 1H) 7.35-7.41 (m, 1H) 7.55 (dd, 1H) 7.83 (dd, 1H) 7.99 (d, 1H)

### Step 1: 4-[3-(2-Chloro-benzyl)-6-fluoro-2-imino-2,3-dihydro-benzoimidazol-1-yl]-6′-methyl-3,4,5,6-tetrahydro-2H-[1,2′]bipyridinyl-3′-ylamine

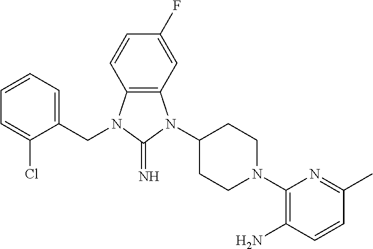 [SMILES: Cc1ccc(N)c(N2CCC(n3c(=N)n(Cc4ccccc4Cl)c4ccc(F)cc43)CC2)n1]

1-(2-Chloro-benzyl)-5-fluoro-3-(6′-methyl-3′-nitro-3,4,5,6-tetrahydro-2H-[1,2′]bipyridinyl-4-yl)-1,3-dihydro-benzoimidazol-2-ylideneamine (Step 2, 40 mg, 0.081 mmol) was dissolved in a mixture of water (10 mL), acetic acid (0.6 mL), and acetonitrile (3 mL). Iron powder (45 mg, 0.81 mmol) was added and the reaction mixture was stirred at 80° C. for 20 min by which time the solution had become pale brown. Saturated aqueous sodium bicarbonate solution (20 mL) was added and the product was extracted with ethyl acetate (3×50 mL). The combined organic extracts were washed with brine (75 mL), dried over MgSO4, filtered, and evaporated to give a brown solid 25 mg (67%) that was used in the next step without any purification. 1H NMR (Methanol-d4) Ppm 2.00 (m, 2H) 2.38 (s, 3H) 2.70-2.83 (m, 2H) 3.01-3.11 (m, 2H) 3.60-3.68 (m, 2H) 4.60-4.72 (m, 1H) 5.43 (s, 2H) 6.78 (d, 1H) 6.90-7.00 (m, 2H) 7.03-7.08 (m, 2H) 7.26-7.31 (m, 1H) 7.32-7.38 (m, 1H) 7.53 (dd, 1H) 7.66 (dd, 1H)

### Step 2: 1-(2-Chloro-benzyl)-5-fluoro-3-(6′-methyl-3′-nitro-3,4,5,6-tetrahydro-2H-[1,2′]bipyridinyl-4-yl)-1,3-dihydro-benzoimidazol-2-ylideneamine

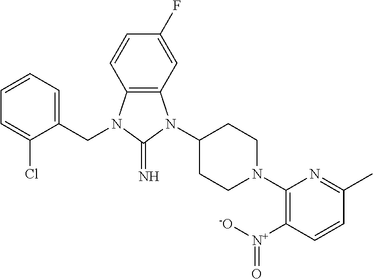

A mixture of 2-fluoro-6-methyl-3-nitro-pyridine (30 mg, 0.192 mmol), 1-(2-chloro-benzyl)-5-fluoro-3-piperidin-4-yl-1,3-dihydro-benzoimidazol-2-ylideneamine (Intermediate AN, 72 mg, 0.202 mmol), and cesium carbonate (75 mg, 0.231 mmol) in DMF (2 mL) was stirred at rt for 30 min. The reaction mixture was poured into water, sat. aq. sodium bicarbonate solution (30 mL) was added and the product was extracted with ethyl acetate (3×50 mL). The combined organic extracts were washed with brine (75 mL), dried over MgSO4, filtered, and evaporated to give a dark green colored solid. The crude residue was purified by reverse-phase preparative HPLC (Method E, gradient from 20% (MeCN +0.1% TFA) in (Water +0.1% TFA) to 80% over 14 minutes) to give the title compound as a green solid, 46 mg (48%). 1H NMR (Methanol-d4) Ppm 2.14 (dd, 2H) 2.54 (s, 3H) 2.61-2.72 (m, 2H) 3.22-3.29 (m, 2H) 4.09-4.15 (m, 2H) 4.74-4.84 (m, 1H) 5.55 (s, 2H) 6.86 (d, 1H) 7.00 (dd, 1H) 7.10 (dt, 1H) 7.27 (dd, 1H) 7.32 (dt, 1H) 7.40 (dt, 1H) 7.53-7.58 (m, 2H) 8.22 (d, 1H)

### IV Biology

The efficacy of the compounds as inhibitors of IGF1-R and InsR tyrosine kinase activity can be demonstrated as follows:

BaF3-Tel-IGF1-R and BaF3-InsR are BaF3 murine proB-cell lymphoma cell derivatives [the BaF3 cell line (also termed Ba/F3) is available from the German Collection of Microorganisms and Cell Cultures (DSMZ), Braunschweig, Germany] that have been rendered IL-3-independent by stable transduction with kinase-activating fusions between human TEL (aa 1-452) and the kinase domain of IGF-1R (aa 976-1367) linked by a Ser-Arg-linker (Boulay et al, Cancer Res 68, 3743-3751, 2008), and a fusion between human TEL (aa 1-337) and the kinase domain (aa 1015-1382) of the Insulin receptor (Melnick J S et al, Proc Natl Acad Sci USA 103, 3153-3158, 2006), respectively. Cells are cultured in RPMI-1640 (Animed #1-14F01-1) supplemented with 2% L-glutamine (Animed #5-10K50-H) and 10% fetal calf serum (FCS, Animed #2-01F16-I). Wild-type, untransfected BaF3 cells are maintained in above medium plus 10 U/ml IL-3 (mouse Interleukin-3, Roche #1380745 or Invitrogen # PMC0035) and are used to identify non-selective, generally growth-inhibitory compounds. Cells (1.5×10⁴ cells per well) are seeded in 190 μl fresh medium into 96-well plates. 10 μl 20× compound solutions are added. As internal control, the kinase inhibitor PKC412 is routinely used. Control cells treated with DMSO (0.1% final concentration) serve as growth reference (set as 100% growth). In addition, a plate blank value is routinely determined in a well containing only 100 μl of medium and no cells. IC₅₀ determinations are performed based on eight 3-fold serial dilutions of the test compound, starting at 10 μM. Following incubation of the cells for 48 h at 37° C. and 5% CO₂, the effect of inhibitors on cell viability is assessed by the resazurin sodium salt dye reduction assay (commercially known as AlamarBlue assay) basically as previously described (O'Brien J. et al., Eur. J. Biochem. 267: 5421-5426, 2000). Briefly, 20 μl dye solution is added per well and the plates incubated for 6 h at 37° C. and 5% CO₂. Thereafter, fluorescence is measured using a Saphirell 96-well plate reader (TECAN, Männedorf, Switzerland) with the following settings: Excitation 544 nm and Emission 590 nm. For data analysis, the plate blank value is subtracted from all data points. The effect of a particular test compound concentration on cell proliferation and viability is expressed as percentage of the blank-corrected reading obtained for cells treated with vehicle only, which is set as 100%. IC₅₀ values can be determined using XLfit (V4.2), applying standard four parameter logistic model #205 (IDBS, Guilford, UK) or other common curve-fitting software.

To achieve a higher throughput, the cell viability assay can also be performed in a 384-well format. Briefly, 4′500 freshly diluted cells are seeded in 54 μl/well into 384-well plates using a liquid dispenser. 6 μl 10× compound solution is added to the cell plate. As internal control, the kinase inhibitor PKC412 is routinely used. Control cells treated with DMSO (0.1% final concentration) serve as growth reference (set as 100% growth). In addition, a plate blank value is routinely determined in a well containing only 60 μl of medium and no cells. Dose-response effects are determined by 3-fold serial compound dilutions, starting at 10 μM. Following incubation of the cells for 48 h at 37° C. and 5% CO₂, the effect of compounds on cell proliferation/viability is assessed by addition of 6 μl resazurin sodium salt dye solution per well. Following incubation for an additional 6 hrs at 37° C. and 5% CO₂, fluorescence is measured using an Infiniti M1000 microplate reader (TECAN, Männedorf, Switzerland) with excitation and emission wavelengths set at 544 and 590 nm, respectively. For data analysis, the plate blank value is subtracted from all data points. IC₅₀ values can be determined by four parameter logistic fitting as described above.

The data obtained are summarized in the table given below:

| Ex. | IC50* | IC50** |
|---|---|---|
| 54 | 405 | |
| 55 | 288 | |
| 56 | | <4.5 |
| 56.1 | | 35 |
| 56.10 | | 88 |
| 56.11 | | 16 |
| 56.12 | | 48 |
| 56.13 | | 9 |
| 56.14 | | 23 |
| 56.15 | | 41 |
| 56.16 | | 39 |
| 56.17 | | 38 |
| 56.18 | | 119 |
| 56.19 | | 382 |
| 56.2 | | 42 |
| 56.3 | | 52 |
| 56.4 | | 100 |
| 56.5 | | 51 |
| 56.6 | 93 | 148 |
| 56.7 | | 69 |
| 56.8 | | 209 |
| 56.9 | | 82 |
| 57 | | 15 |
| 58 | | 37 |
| 59 | | 407 |
| 60 | | 64 |
| 61 | | 8 |
| 62 | | 36 |
| 63 | | 22 |
| 64 | | 163 |
| 65 | | 5 |
| 66 | | 180 |
| 67 | | 23 |
| 67.1 | | 10 |
| 67.2 | | 62 |
| 67.3 | | 167 |
| 67.4 | | 1010 |
| 67.5 | | 30 |
| 67.6 | | 517 |
| 67.7 | | 644 |
| 77 | | 68 |
| 78 | | 62 |

*IC50 96-well plate format [nmol l−1];
**IC50 384-well plate format [nmol l−1]

### V Pharmaceutical Formulations

Tablets

Tablets comprising 50 mg of active ingredient, of the compounds of formula (I) described in Examples 1 to 88, and having the following composition are prepared in customary manner:

Composition:

| | | |
|---|---|---|
| active ingredient | 50 | mg |
| wheat starch | 150 | mg |
| lactose | 125 | mg |
| colloidal silicic acid | 12.5 | mg |
| talc | 22.5 | mg |
| magnesium stearate | 2.5 | mg |
| Total: | 362.5 | mg |

Preparation: The active ingredient is mixed with a portion of the wheat starch, with the lactose and the colloidal silicic acid and the mixture is forced through a sieve. A further portion of the wheat starch is made into a paste, on a water bath, with five times the amount of water and the powder mixture is kneaded with the paste until a slightly plastic mass is obtained. The plastic mass is pressed through a sieve of about 3 mm mesh size and dried, and the resulting dry granules are again forced through a sieve. Then the remainder of the wheat starch, the talc and the magnesium stearate are mixed in and the mixture is compressed to form tablets weighing 145 mg and having a breaking notch.

Soft Capsules

5000 soft gelatin capsules comprising each 50 mg of active ingredient, for example one of the compounds of formula (I) described in Examples 1 to 88, are prepared in customary manner:

Composition:

| | | |
|---|---|---|
| active ingredient | 250 | g |
| Lauroglykol | 2 | litres |

Preparation: The pulverized active ingredient is suspended in Lauroglykol® (propylene glycol laurate, Gattefossé S. A., Saint Priest, France) and ground in a wet pulverizer to a particle size of approx. 1 to 3 μm. 0.419 g portions of the mixture are then dispensed into soft gelatin capsules using a capsule-filling machine.

1. A compound of formula (I) 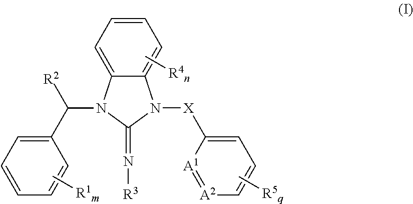
or a salt thereof, wherein
m represents 0, 1, 2, 3 or 4;
n represents 0, 1, 2, 3 or 4;
q represents 0, 1, 2 or 3;
X represents a group 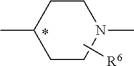
wherein the atom marked * is bound to the imidazole;
A¹ represents N, CH or CR⁵;
A² represents N, CH or CR⁵;
R¹ represents halogen, C₁₋₇alkyl, C₁₋₇alkyoxy, halo-C₁₋₇alkyl or halo-C₁₋₇alkyoxy; and/or
R¹ represents, provided two substituents R¹ are in vicinal position, together with the carbon atoms to which they are attached a cyclic moiety, said moiety (a) being saturated or partly saturated, (b) contains 5-8 ring forming atoms, (c) contains 0-3 nitrogen atoms, 0-2 oxygen atoms, and 0-2 sulfur atoms, and (d) is unsubstituted or substituted, the substituents being selected from the group consisting of halogen, C₁₋₇alkyl, C₁₋₇alkyoxy, halo-C₁₋₇alkyl and halo-C₁₋₇alkyoxy;
R² represents hydrogen, halogen, C₁₋₇alkyl or halo-C₁₋₇alkyl;
R³ represents hydrogen, C₁₋₇alkyl, halo-C₁₋₇alkyl, C₁₋₇alkyl-carbonyl, halo-C₁₋₇alkyl-carbonyl, C₁₋₇alkoxy-carbonyl, or halo-C₁₋₇alkoxy-carbonyl;
R⁴ represents halogen, C₁₋₇alkyl, C₁₋₇alkoxy, halo-C₁₋₇alkyl or halo-C₁₋₇alkoxy;
R⁵ represents a substituent different from hydrogen, said substituent (a) having 1-50 atoms selected from the group consisting of hydrogen, carbon, halogen and hetero atoms and (b) being bound via a single bond;
R⁶ represents hydrogen, hydroxy, halogen, C₁₋₇alkyl, C₁₋₇alkyoxy, halo-C₁₋₇alkyl or halo-C₁₋₇alkyoxy.

2. The compound according to claim 1, or a salt thereof,
depicted by formula (I-1) 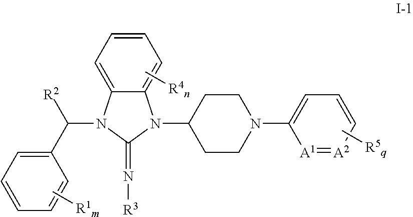
or depicted by formula (I-2)

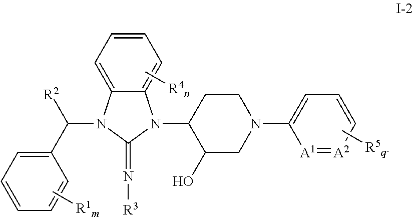

3. The compound according to claim 1, or a salt thereof, depicted by formula (I-3) 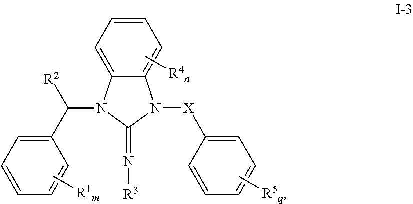
or depicted by formula (I-4)

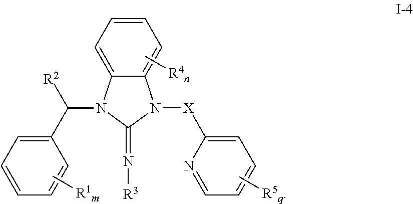

4. The compound according to claim 1, or a salt thereof, depicted by formula I-5 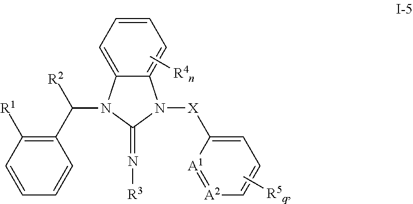
or depicted by formula (I-6) 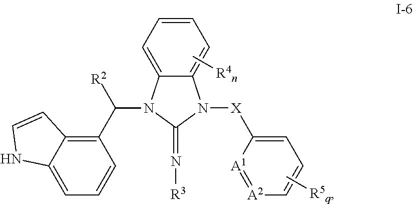
or depicted by formula (I-7) 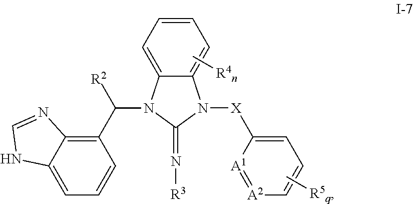
or depicted by formula (I-8)

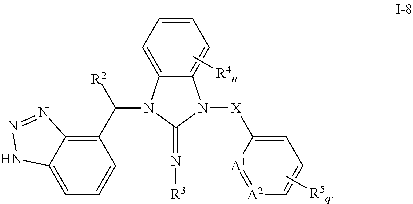

5. The compound according to claim 1, or a salt thereof, depicted by formula I-9 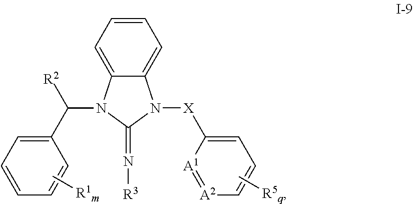
or depicted by formula (I-10)

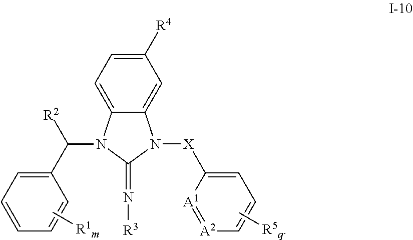

6. The compound according to claim 1, or a salt thereof, wherein
R⁵ represents a group —X′—R⁵′ in which
X′ represents either a single bond or a linker selected from the group consisting of 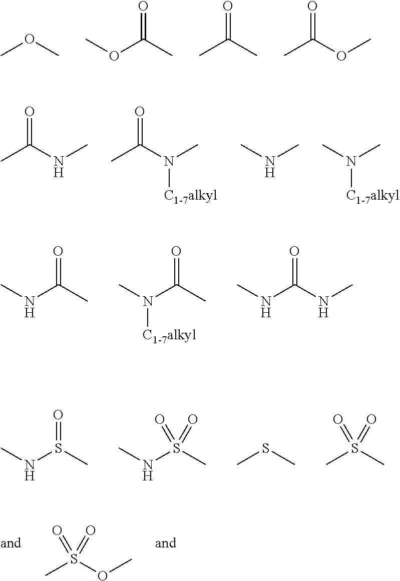
R⁵′ represents hydroxy, halo, cyano, carboxy, aminocarbonyl, amino, or optionally substituted C₁₋₇alkyl, optionally substituted C₃₋₁₂cycloalkyl, optionally substituted C₆₋₂₀aryl, optionally substituted heterocyclyl having 5-10 ring atoms, and optionally substituted heteroaryl having 5-10 ring atoms, the optional substituents being selected from the group consisting of hydroxy, halo, cyano, carboxy, aminocarbonyl, amino, C₁₋₇alkylamino, di(C₁₋₇alkyl)amino, C₁₋₇alkyl, and C₁₋₇alkyloxy.

7. The compound according to claim 1,
or a salt thereof, wherein
R⁵ represents methyl, methoxy, acetylamino, chloro, cyano, or trifluoromethyl.

8. A compound according to claim 1, or a salt thereof, wherein
q represents 2, the substituents R⁵ being located in the 2- and 5-position or
q represents 1, the substituent R⁵ being located in the 2- or 3-position.

9. The compound according to claim 1, or a salt thereof, wherein
R¹ represents halogen or
R¹ represents, together with the phenyl ring, an unsubstituted or substituted indolyl, isoindolyl, indazolyl, benzimidazolyl, benztriazolyl, chinolinyl, isochinnolinyl, cinnolinyl, phtalazinyl, chazolinyl, chinoxalinyl, naphtalenyl, tetrahydro-naphtalenyl, indenyl, dihydroindenyl, the substituents being selected from the group consisting of halogen.

10. The compound according to claim 1, or a salt thereof, wherein
R³ represents hydrogen, C₁₋₇alkyl-carbonyl or C₁₋₇alkyloxy-carbonyl.

11. A pharmaceutical composition comprising a therapeutically effective amount of a compound of formula (I) according to claim 1, or a pharmaceutically acceptable salt thereof, and one or more pharmaceutically acceptable carriers.

12. The pharmaceutical composition of claim 11 further comprising one or more therapeutically active agents, selected from antiproliferative agents.

13-17. (canceled)

18. A method of modulating IGF-1R activity in a subject, comprising the step of administering to a subject a therapeutically effective amount of a compound of formula (I) according to claim 1, or a pharmaceutically acceptable salt thereof.

19. A method for the treatment of an IGF-1R mediated disorder or disease comprising the step of administering to a subject a therapeutically effective amount of a compound of formula (I) according to claim 1, or a pharmaceutically acceptable salt thereof.

20. The method of claim 19, wherein said IGF-1R mediated disorder or disease is selected from the group consisting of multiple myeloma, neuroblastoma, synovial, hepatocellular, Ewing's Sarcoma, and adrenocotical carcinoma or is a solid tumor selected from the group consisting of osteosarcoma, melanoma, tumor of breast, renal, prostate, colorectal, thyroid, ovarian, pancreatic, lung, uterine, and gastrointestinal tumor or is acute lung injury or pulmonary fibrosis.
